(19) United States
(12) Patent Application Publication
(10) Pub. No.: US 2024/0075114 A1
(43) Pub. Date: Mar. 7, 2024

(54) IMMUNOGENIC PRODUCTS BASED ON MUTEIN AMYLOID beta (Abeta) AMINO ACID SEQUENCES AND USES THEREOF

(71) Applicant: ABBVIE DEUTSCHLAND GMBH & CO. KG, Wiesbaden (DE)

(72) Inventors: Stefan Barghorn, Mannheim (DE); Simone Giaisi, Ludwigshafen (DE); Heinz Hillen, Ludwigshafen (DE); Andreas Striebinger, Ludwigshafen (DE)

(21) Appl. No.: 18/050,314

(22) Filed: Oct. 27, 2022

Related U.S. Application Data
(63) Continuation of application No. 14/792,500, filed on Jul. 6, 2015, now abandoned.
(60) Provisional application No. 62/021,308, filed on Jul. 7, 2014.

(51) Int. Cl. A61K 39/00; C07K 7/06; C07K 7/08; C07K 14/47; C07K 16/18; G01N 33/68

(52) U.S. Cl. CPC A61K 39/0007; C07K 7/06; C07K 7/08; C07K 14/4711; C07K 16/18; G01N 33/6896; A61K 2039/55505; A61K 2039/55566; C07K 2317/33; C07K 2317/34; G01N 2333/4709; G01N 2800/2821

(57) ABSTRACT

The invention relates to immunogenic products based on mutein amyloid β (Aβ) amino acid sequences, in particular to oligomers of Aβ muteins, and to the use of said products in diagnosis, treatment and prevention of conditions such as amyloidoses, and for identifying agents capable of binding to said products.

### CROSS-REFERENCE TO RELATED APPLICATION(S)

This application is a continuation of U.S. patent application Ser. No. 14/792,500, filed Jul. 6, 2015, now abandonded, which claims priority to U.S. Provisional Patent Application No. 62/021,308, filed on Jul. 7, 2014, the contents of all of which are fully incorporate herein by reference.

### SEQUENCE LISTING

The instant application contains a Sequence Listing which has been submitted in ST.26 XML format via Patent Center and is hereby incorporated by reference in its entirety. Said ST.26 XML copy, created on Oct. 27, 2022, is named “12075USC1 Sequence Listing.xml”, and is 38,886 bytes in size.

### FIELD OF THE INVENTION

The invention relates to immunogenic products based on mutein amyloid β (Aβ) amino acid sequences, in particular to oligomers of Aβ muteins, and to the use of said products in diagnosis, treatment and prevention of conditions such as amyloidoses, and for identifying agents capable of binding to said products.

### BACKGROUND OF THE INVENTION

Alzheimer's disease (AD) is a neurodegenerative disorder characterized by a progressive loss of cognitive abilities and by characteristic neuropathological features comprising deposits of amyloid beta (Aβ) peptide, neurofibrillary tangles and neuronal loss in several regions of the brain (Hardy and Selkoe, Science 297: 353, 2002; Mattson, Nature 431: 7004, 2004. Cerebral amyloid deposits and cognitive impairments very similar to those observed in Alzheimer's disease are also hallmarks of Down syndrome (trisomy 21), which occurs at a frequency of about 1 in 800 births.

The Aβ peptide arises from the amyloid precursor protein (APP) by proteolytic processing. This processing is effected by the cooperative activity of several proteases named α-, β- and γ-secretase and leads to a number of specific fragments of differing length. The Δβ peptide deposits consist mostly of peptides with a length of 40 or 42 amino acids (Aβ(1-40), Aβ(1-42)). This protein, which tends to polymerize in an aqueous environment, may be present in very different molecular forms including insoluble forms, such as Aβ fibrils, as well as soluble forms, such as Aβ oligomers.

A simple correlation of the deposition of insoluble protein with the occurrence or progression of dementia disorders such as, for example, Alzheimer's disease, has proved to be unconvincing (Terry et al., Ann. Neurol. 30: 572-580, 1991; Dickson et al., Neurobiol. Aging 16: 285-298, 1995). In contrast, the loss of synapses and cognitive perception seems to correlate better with soluble Aβ forms (Lue et al., Am J Pathol 155: 853-862, 1999; McLean et al., Ann Neurol 46: 860-866, 1999).

Soluble Aβ oligomers have been generated synthetically (Barghorn et al., J Neurochem 95: 834-847, 2005), harvested from APP-transfected cell cultures (Walsh et al., Nature 416: 535-539, 2002) and isolated from the brain of APP-transgenic mice (Lesne et al., Nature 440: 352-357, 2006). WO 2004/067561 refers to globular oligomers (“globulomers”) of Aβ(1-42) peptide and a process for preparing them. WO 2006/094724 relates to non-diffusible globular Aβ(X-38 . . . 43) oligomers wherein X is selected from the group consisting of numbers 1 . . . 24. WO 2004/067561 and WO 2006/094724 further describes that limited proteolysis of the globulomers yields truncated versions of said globulomers such as Aβ(20-42) or Aβ(12-42) globulomers. WO 2007/064917 describes the cloning, expression and isolation of recombinant forms of amyloid β peptide (referred to hereafter as N-Met Aβ(1-42)) and globulomeric forms thereof.

The data suggest the existence of an amyloid fibril independent pathway of Aβ folding and assembly into Aβ oligomers which display one or more unique epitopes (hereinafter referred to as the globulomer epitopes). Said globulomer epitopes were detected in the brain of AD patients and APP transgenic mice, and Aβ globulomer was found to bind specifically to neurons and to block hippocampal long-term potentiation. It has been found that soluble Aβ globulomer exert its detrimental effects essentially by interaction with the P/Q type presynaptic calcium channel, and that inhibitors of this interaction are therefore useful for treatment of amyloidoses such as Alzheimer's disease (WO 2008/104385).

Monoclonal antibodies capable of discriminating between soluble Aβ globulomers and other Aβ species such as monomers and fibrils have been described previously, for example in WO 2007/062852. Monoclonal antibodies selective for Aβ globulomers were shown to prevent pathological effects of Aβ oligomers in vitro and in vivo (Hillen et al., J Neurosci 30(31): 10369-10379, 2010). These results indicate that antibody-effected neutralization of Aβ globulomers is efficient in a preclinical AD model, and may thus also be efficient in the treatment and prevention of AD and other amyloidoses.

Besides monoclonal anti-Aβ antibodies for passive immunization, the use of Aβ preparations for active immunization has been a matter of AD research. Results of such research corroborate that the therapeutic index of an Aβ oligomer vaccine is dependent on its ability to elicit immune responses that are specific for the pathogenic Aβ species. The importance of specificity is corroborated by the results of previous vaccination studies. The use of pre-aggregated Aβ(1-42) in a clinical study of active immunization on Alzheimer patients, for example, resulted in considerable side effects (meningoencephalitis, hemorrhages) in some of the patients since the antibodies formed also recognized the Aβ(1-42) forms presumably required for cell lining, resulting in inflammatory reactions (D. Schenk; Nat. Rev. Neurosci. 3, 824-828 (2002)).

An Aβ oligomer vaccine should of course not only avoid eliciting autoantibodies binding to other than the pathogenic Aβ forms but in general should not induce the formation of autoantibodies that are capable of pathogenic cross-reactions. In certain cases it has, however, been found that monoclonal antibodies identified using preparations of wild type Aβ oligomers may exhibit cross-reactivity to platelet factor 4 (PF-4). PF-4 binds to heparin, thus forming neo-epitopes. This may elicit an immune response resulting in a required thrombotic disorder known as heparin-induced thrombocytopenia (HIT). HIT is known to be trigged by heparin treatment. Nevertheless, the symptoms of HIT, thrombocytopenia and thrombosis, as well as anti-PF-4 autoantibodies have also been observed in patients without prior administration of heparin (Warkentin et al., Am J Med 121(7): 632-6, 2008).

There exists a tremendous, unmet therapeutic need for the development of an Aβ oligomer vaccine that is effective against Alzheimer's disease and related disorders, while not inducing negative and potentially lethal side effects such as pathologic autoimmune responses on the patient's body. Such a need is particularly evident in view of the increasing longevity of the general population and, with this increase, an associated rise in the number of patients annually diagnosed with Alzheimer's disease or related disorders.

### SUMMARY OF THE INVENTION

The present invention meets said need by providing a novel immunogenic product that is capable of eliciting an antiserum which specifically binds to Aβ globulomer epitopes, but has no or low cross-reactivity to platelet factor 4 (PF-4). Accordingly the novel immunogenic product comprises one or more epitopes which are recognized by globulomer-specific antibodies. Monoclonal antibodies binding such epitopes include 7C6, 4D10 and 5F7 which have been described in WO 2007/062852 and are obtainable from the hybridomas designated by American Type Culture Collection deposit numbers PTA-7240, PTA-7405, and PTA-7241, respectively.

Thus, the invention provides an immunogenic product comprising an amyloid β (Aβ) amino acid sequence having 62.5% or higher identity to the amino acid sequence

[SEQ ID NO: 2; Aβ(18-33)]
V₁₈F₁₉F₂₀A₂₁E₂₂D₂₃V₂₄G₂₅S₂₆N₂₇K₂₈G₂₉A₃₀I₃₁I₃₂G₃₃,

wherein the product
i) is reactive with a monoclonal antibody selected from the group consisting of monoclonal antibody 7C6 obtainable from a hybridoma designated by American Type Culture Collection deposit number PTA-7240; monoclonal antibody 4D10 obtainable from a hybridoma designated by American Type Culture Collection deposit number PTA-7405, or monoclonal antibody 5F7 obtainable from a hybridoma designated by American Type Culture Collection deposit number PTA-7241; and
ii) is capable of eliciting a polyclonal antiserum which has no or low cross-reactivity to platelet factor 4 (PF-4)

The present invention also relates to a composition comprising an immunogenic product as disclosed herein.

The present invention further relates to a method of treating or preventing an amyloidosis in a subject in need thereof, which comprises administering an immunogenic product as disclosed herein to the subject. In a related aspect, the present invention relates to an immunogenic product as disclosed herein for use in treating or preventing an amyloidosis.

The present invention also relates to a method of diagnosing an amyloidosis which comprises providing a sample from the subject suspected of having the amyloidosis, contacting the sample with an immunogenic product as disclosed herein for a time and under conditions sufficient for the formation of a complex comprising the product and an antibody, the presence of the complex indicating the subject has the amyloidosis. In a related aspect, the present invention relates to an immunogenic product as disclosed herein for use in diagnosing an amyloidosis.

The present invention also relates to a method of identifying an agent capable of binding to an immunogenic product as disclosed herein, which method comprises the steps of: a) exposing one or more agents of interest to the product for a time and under conditions sufficient for the one or more agents to bind to the product; and b) identifying those agents which bind to the product.

In a related aspect, the invention provides a method of providing an antibody capable of binding to an immunogenic product as disclosed herein, which comprises
i) providing an antigen comprising the product;
ii) exposing an antibody repertoire to said antigen; and
iii) selecting from said repertoire an antibody which binds to the product.

The present invention further relates to a molecule comprising an amino acid sequence identical to a portion (X—Y) of an amino acid sequence selected from the group consisting of:

[SEQ ID NO: 13; Aβ(1-43)F19A]
D₁A₂E₃F₄R₅H₈D₇S₈G₉Y₁₀E₁₁V₁₂H₁₃H₁₄Q₁₅K₁₈L₁₇V₁₈A₁₉F₂₀A₂₁E₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 14; Aβ(1-43)F20A]
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 15; Aβ(1-43)E22A]
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 16; Aβ(1-43)E22F]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁F₂₂D₂₃V₂₄G₂₅S₂₆N₂₇K₂₈
G₂₉A₃₀I₃₁I₃₂G₃₄L₃₄M₃₅V₃₆G₃₇G₃₈V₃₉V₄₀I₄₁A₄₂T₄₃;
[SEQ ID NO: 17; Aβ(1-43)E22V]
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁V₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 18; Aβ(1-43)E22L]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 19; Aβ(1-43)D23K]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂K₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 20; Aβ(1-43)D23L]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂L₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 21; Aβ(1-43)G25V]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄V₂₅S₂₆N₂₇K₂₈
G₂₉A₃₀I₃₁I₃₂G₃₃L₃₄M₃₅V₃₆G₃₇G₃₈V₃₉V₄₀₁₄₁A₄₂T₄₃;
[SEQ ID NO: 22; Aβ(1-43)A30G]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄G₂₅S₂₆N₂₇K₂₈
G₂₉G₃₀I₃₁I₃₂G₃₃L₃₄M₃₅V₃₆G₃₇G₃₈V₃₉V₄₀I₄₁A₄₂T₄₃;
[SEQ ID NO: 23; Aβ(1-43)F20G E22A]
D₁A₂E₃F₄R₅H₆D₇S₈G₉Y₁₀E₁₁V₁₂H₁₃H₁₄Q₁₅K₁₆L₁₇V₁₈F₁₉G₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 24; Aβ(1-43)F20A I31A]
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀A₃₁I₃₂G₃₃L₃₄M₃₅V₃₆G₃₇G₃₈V₃₉V₄₀I₄₁A₄₂T₄₃;
[SEQ ID NO: 25; Aβ(1-43)F20C I31C]
D₁A₂E₃F₄R₅H₆D₇S₈G₉Y₁₀E₁₁V₁₂H₁₃H₁₄Q₁₅K₁₆L₁₇V₁₈F₁₉C₂₀A₂₁E₂₂D₂₃V₂₄G₂₅S₂₆N₂₇K₂₈
G₂₉A₃₀C₃₁I₃₂G₃₃L₃₄M₃₅V₃₆G₃₇G₃₈V₃₉V₄₀I₄₁A₄₂T₄₃;
[SEQ ID NO: 26; Aβ(1-43)A21Q E22L]
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 27; Aβ(1-43)A21L E22Q]
D₁A₂E₃F₄R₅H₆D₇S₈G₉Y₁₀E₁₁V₁₂H₁₃H₁₄Q₁₅K₁₆L₁₇V₁₈F₁₉F₂₀L₂₁Q₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 28; Aβ(1-43)A21Q D23N]
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁E₂₂N₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 29; Aβ(1-43)E22A G25A]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄A₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
and
[SEQ ID NO: 30; Aβ(1-43)E22A S26A]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄G₂₅A₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,

with X being selected from the group consisting of the numbers 1 . . . 18, 4 . . . 18, 12 . . . 18, or is 18, and Y being selected from the group consisting of the numbers 33 . . . 43, 33 . . . 42, 33 . . . 41, or 33 . . . 40; or a crosslinked derivative thereof, wherein at least 2 non-contiguous residues of the amino acid sequence are covalently linked with each other.

### BRIEF DESCRIPTION OF THE FIGURES

FIG. 1 shows a size exclusion chromatogram (SEC) on Superose 12 HR 10/300 GL of A) wild type Aβ(20-42) globulomer; B) truncated Aβ E22A mutein oligomer; and C) truncated Aβ F20G E22A mutein oligomer.

FIG. 2 is a table indicating whether truncated forms of the listed Aβ mutein oligomers were found to be reactive with murine, Aβ(20-42) globulomer-reactive, monoclonal antibodies m7C6 and m4D10 in an ELISA (+++: strong reactivity; ++: good reactivity; +: moderate reactivity; +/−: no or little reactivity).

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or”, unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, protein and nucleic acid chemistry, and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The present invention provides an immunogenic product that is on the one hand reactive with antibodies binding globulomer epitopes such as monoclonal antibody 7C6, monoclonal antibody 4D10, or monoclonal antibody 5F7, and on the other hand is capable of eliciting a polyclonal antiserum which has no or low cross-reactivity to PF-4.

PF-4 is a small, 70-amino acid cytokine that belongs to the CXC chemokine family and is also known as chemokine (C—X—C motif) ligand 4 (CXCL4). PF-4 is released from alpha-granules of activated platelets during platelet aggregation, and promotes blood coagulation by moderating the effects of heparin-like molecules. Due to these functions, it is predicted to be involved in wound repair and inflammation (Eismann et al., Blood 76(2): 336-44, 1990). PF-4 is usually found in a complex with proteoglycan and can form complexes with the anticoagulant heparin which is in use as pharmacological treatment of thrombosis. It has a well described pathological function in heparin-induced thrombocytopenia (HIT), an idiosyncratic autoimmune reaction to the administration of the anticoagulant heparin (Warkentin, N. Engl. J. Med. 356(9): 891-3, 2007), wherein the heparin:PF4 complex is the antigen. PF4 autoantibodies have also been found in patients with thrombosis and features resembling HIT but no prior administration of heparin (Warkentin et al., Am. J. Med. 121(7): 632-6, 2008). Heparin-induced thrombocytopenia is characterized by the development of thrombocytopenia (a low platelet count), and in addition HIT predisposes to thrombosis. In view of these functions and involvement of PF-4 in pathological processes it can be concluded that the administration of antigens (e.g. vaccines) which elicite a polyclonal antiserum which show binding (e.g. cross-reactivity) to the PF-4 present in a subject may affect said PF-4 functions and thus result in adverse (side) effects. The degree and nature of such adverse effects may vary depending on parameters such as location and size of the epitope on PF-4, binding strength and nature of the respective antiserum.

The immunogenic product of the invention is capable of eliciting a polyclonal antiserum which has no or low cross-reactivity to platelet factor 4 (PF-4). Thus, a development of adverse reactions such as HIT which might result from immunization with products having PF-4 cross-reactivity can be avoided when using the immunogenic product of the invention for active immunization of a subject.

In one aspect of the invention, the polyclonal antiserum having no or low cross-reactivity to PF-4 elicited by the immunogenic product described herein is a polyclonal antiserum from a mouse or a rabbit. Preferably, the polyclonal antiserum is an affinity purified antiserum enriched in antibodies binding to the product.

In different aspects of the invention, the PF-4 is selected from the PF-4 in cynomolgus plasma and the PF-4 in human plasma.

The capability of an immunogenic product of eliciting a polyclonal antiserum which has no or low cross-reactivity to platelet factor 4 (PF-4) may be tested using standard methods well known in the art. For example, the immunogenic product may be used to immunize a mouse or a rabbit in order to subsequently obtain a polyclonal antiserum thereof. As generally known, among individual antisera there may be variations regarding the amount of antibodies raised against the immunogenic product used for immunization. In order to avoid false negative results in the assay for PF-4 reactivity, the polyclonal antiserum may be therefore be enriched in antibodies binding to the immunogenic product. Such enrichment can be achieved using standard methods of affinity purification which, for example, comprise immobilizing the immunogenic product on a solid carrier (e.g. sepharose beads), contacting the carrier with the antiserum such as to allow binding of antibodies to the immobilized immunogenic product, and eluting bound antibodies from the carrier (e.g. using an acidic elution buffer), wherein the eluate is the affinity purified antiserum enriched in antibodies binding to the immunogenic product. If the immobilized product is a (truncated) Aβ mutein oligomer the Aβ mutein comprised in the immunogenic product may optionally be additionally immobilized on the carrier in monomeric form in order to ensure that all anti-Aβ antibodies are affinity purified which potentially may also bind to non-oligomeric Aβ forms such as monomeric or fibrillary forms.

In a specific aspect of the invention, the cross-reactivity is determined as the binding of the polyclonal antiserum to plasma PF-4, with the polyclonal antiserum being immobilized, for example by binding to immobilized anti-IgG antibody. Bound PF-4 may be detected as anti-PF-4 antibody bound to said PF-4. The anti-PF-4 antibody may be a monoclonal antibody or a polyclonal antiserum and in particular is reactive with PF-4 having the amino acid sequence EAEEDGDLQCLCVKTTSQVRPRHITSLEVIKAGPHCPTAQLIATLKNGRKICLDLQAPL YKKIIKKLLES (SEQ ID NO:12). The anti-PF-4 antibody may be a labeled antibody and detection is measuring the signal generated by the label. Alternatively, the bound anti-PF-4 antibody may be detected as labeled anti-IgG antibody bound to anti-PF-4 antibody and detection is measuring the signal generated by the label.

According to another particular embodiment, the cross-reaction to PF-4 of antisera elicited by the immunogenic product of the invention refers to ratio of AUC values for said antisera and a reference anti-PF-4 antibody obtained by (i) performing an aligned sandwich-ELISA with human or cynomolgus plasma and dilution series of binding protein and reference anti-PF-4 antibody, (ii) plotting detected signal (y-axis) against log-transformed concentrations of antiserum or reference anti-PF-4 antibody (x-axis), and (iii) determining the area under the curve (AUC, or total peak area) from these non-curve fitted data in the measured range.

A “reference anti-PF-4 antibody”, as used herein, is an antibody, in particular a monoclonal antibody, that is specifically reactive with PF-4, in particular human (HPF4). Such an antibody is obtainable by providing an antigen comprising human PF-4, for instance human PF-4 having amino acid sequence EAEEDGDLQCLCVKTTSQVRPRHITSLEVIKAPHCPTAQLIATLKNGRKICLDLQAPLY KKIIKKLLES (SEQ ID NO:12), exposing an antibody repertoire to said antigen and selecting from said antigen repertoire an antibody which binds specifically to human PF-4. The antibody may optionally be affinity purified using the immunogen (human PF-4). Such reference anti-PF4 antibodies are commercially available, for example, monoclonal anti-HPF4 antibody, Abcam cat. no.: ab49735.

In a particular aspect of the invention, the immunogenic product described herein is capable of eliciting a polyclonal antiserum having a cross-reactivity to PF-4 that is at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times smaller than the cross-reactivity of a reference anti-PF-4 antibody to PF-4.

Further, the immunogenic products of the invention are characterized by their reactivity with particular antibodies. Such antibodies include in particular antibodies binding globulomer epitopes, in particular antibodies having a binding affinity to an Aβ(20-42) globulomer that is greater than the binding affinity of the antibody to an Aβ(1-42) globulomer.

Antibodies having a binding affinity to an Aβ(20-42) globulomer that is greater than the binding affinity of the antibody to an Aβ(1-42) globulomer are described in WO 2007/062852, which are incorporated herein by reference, and include, for instance, a monoclonal antibody selected from the group consisting of 7C6, 4D10 und 5F7.

Thus, according to one embodiment of the invention, the immunogenic product of the invention is reactive with a monoclonal antibody selected from the group consisting of monoclonal antibody 7C6 obtainable from a hybridoma designated by American Type Culture Collection deposit number PTA-7240; or monoclonal antibody 4D10 obtainable from a hybridoma designated by American Type Culture Collection deposit number PTA-7405, or monoclonal antibody 5F7 obtainable from a hybridoma designated by American Type Culture Collection deposit number PTA-7241.

In one aspect of the invention, the monoclonal antibody 7C6 binds to the immunogenic product described herein with high affinity, for instance with a K_{D} of 1×10⁻⁶ M or greater affinity or with a K_{D} of 1×10⁻⁷ M or greater affinity, e.g. with a K_{D} of 3×10⁻⁸ M or greater affinity, with a K_{D} of 1×10⁻⁸ M or greater affinity, e.g. with a K_{D} of 3×10⁻⁹ M or greater affinity, with a K_{D} of 1×10⁻⁹ M or greater affinity, e.g. with a K_{D} of 3×10⁻¹⁰ M or greater affinity, with a K_{D} of 1×10⁻¹⁰ M or greater affinity, e.g. with a K_{D} of 3×10⁻¹¹ M or greater affinity, or with a K_{D} of 1×10⁻¹¹ M or greater affinity.

In another aspect of the invention, the monoclonal antibody 4D10 binds to the immunogenic product described herein with high affinity, for instance with a K_{D} of 1×10⁻⁶ M or greater affinity or with a K_{D} of 1×10⁻⁷ M or greater affinity, e.g. with a K_{D} of 3×10⁻⁸ M or greater affinity, with a K_{D} of 1×10⁻⁸ M or greater affinity, e.g. with a K_{D} of 3×10⁻⁹ M or greater affinity, with a K_{D} of 1×10⁻⁹ M or greater affinity, e.g. with a K_{D} of 3×10⁻¹⁰ M or greater affinity, with a K_{D} of 1×10⁻¹⁰ M or greater affinity, e.g. with a K_{D} of 3×10⁻¹¹ M or greater affinity, or with a K_{D} of 1×10⁻¹¹ M or greater affinity.

In still another aspect of the invention, the monoclonal antibody 5F7 binds to the immunogenic product described herein with high affinity, for instance with a K_{D} of 1×10⁻⁶ M or greater affinity or with a K_{D} of 1×10⁻⁷ M or greater affinity, e.g. with a K_{D} of 3×10⁻⁸ M or greater affinity, with a K_{D} of 1×10⁻⁸ M or greater affinity, e.g. with a K_{D} of 3×10⁻⁹ M or greater affinity, with a K_{D} of 1×10⁻⁹ M or greater affinity, e.g. with a K_{D} of 3×10⁻¹⁰ M or greater affinity, with a K_{D} of 1×10⁻¹⁰ M or greater affinity, e.g. with a K_{D} of 3×10⁻¹¹ M or greater affinity, or with a K_{D} of 1×10⁻¹¹ M or greater affinity.

The immunogenic products of the present invention that react with globulomer-specific antibodies are believed to display at least one globulomer epitope. Therefore, the immunogenic products of the present invention are capable of eliciting an immune response having a similar profile as the immune response elicited when Aβ(20-42) globulomers or other truncated globulomers are used as immunogen.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by a binding protein, in particular by an antibody. In certain embodiments, a binding protein or an antibody is said to specifically bind an antigen when it preferentially recognizes its target antigen in a complex mixture of proteins and/or macromolecules.

According to a particular embodiment, the immunogenic products of the invention are characterized by their ability of eliciting such a particular immune response, for instance if a mammal, e.g. a rabbit or a mouse, is immunized with an immunogenic product of the invention.

An immune response may be regarded as a mixture of antibodies resulting from challenging (immunizing) a host with an antigen (the immunogen). Said mixture of antibodies can be obtained from the host and is herein referred to as the polyclonal antiserum.

In one aspect, such a particular immune response, i.e., the corresponding polyclonal antiserum, is characterized by comprising an antibody having a binding affinity to an immunogenic product of the invention or to an Aβ globulomer that is greater than the binding affinity of the antibody to at least one Aβ form selected from the group consisting of monomeric Aβ(1-42), monomeric Aβ(1-40), monomeric Aβ(20-42), fibrillomeric Aβ(1-42), and fibrillomeric Aβ(1-40), and preferably to all of said Aβ forms.

According to a particular embodiment, the immune response, i.e., the corresponding polyclonal antiserum, is characterized by having an affinity to an immunogenic product of the invention or to an Aβ globulomer which is at least 2 times, e. g. at least 3 times or at least 5 times, preferably at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times greater than the binding affinity of the antiserum to at least one Aβ form selected from the group consisting of monomeric Aβ(1-42), monomeric Aβ(1-40), monomeric Aβ(20-42), fibrillomeric Aβ(1-42), and fibrillomeric Aβ(1-40), and preferably to all of said Aβ forms.

In related aspects of the invention, said Aβ globulomer is selected from the group consisting of an Aβ(1-42) globulomer, an Aβ(12-42) globulomer and an Aβ(20-42) globulomer.

As used herein, the ellipsis A . . . B denotes the set comprising all natural numbers from A to B, including both, e.g. “17 . . . 20” thus denotes the group of the numbers 17, 18, 19 and 20. The hyphen denotes a contiguous sequence of amino acids, i.e., “X—Y” comprises the sequence from amino acid X to amino acid Y, including both. Thus, “A . . . B-C . . . D” comprises all possible combinations between members of these two sets, e.g. “17 . . . 20-40 . . . 42” comprises all of the following: 17-40, 17-41, 17-42, 18-40, 18-41, 18-42, 19-40, 19-41, 19-42, 20-40, 20-41 and 20-42.

Unless stated otherwise, all numbers refer to the beginning of the mature peptide, 1 indicating the N-terminal amino acid.

The term “Aβ(X—Y)” as used herein refers to a polypeptide having the amino acid sequence from amino acid position X to amino acid position Y of the human amyloid beta (Aβ) protein including both X and Y, in particular to the amino acid sequence from amino acid position X to amino acid position Y of the amino acid sequence D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄G₂₅S₂₆N₂₇K₂₈G₂₉A₃₀I₃₁I₃₂G₃₃L₃₄M₃₅V₃₆G₃₇G₃₈V₃₉V₄₀I₄₁A₄₂T₄₃ (SEQ ID NO:1) (corresponding to amino acid positions 1 to 43 of human Aβ protein), or a mutein thereof.

The term “Aβ(X—Y) monomer” or “monomeric Aβ(X—Y)” here refers to the isolated form of the Aβ(X—Y) peptide, preferably a form of the Aβ(X—Y) peptide which is not engaged in essentially non-covalent interactions with other Aβ peptides. Practically, the Aβ(X—Y) monomer is usually provided in the form of an aqueous solution. In a particularly preferred embodiment of the invention, the aqueous monomer solution contains 0.05% to 0.2%, more preferably about 0.1% NH₄OH. In another particularly preferred embodiment of the invention, the aqueous monomer solution contains 0.05% to 0.2%, more preferably about 0.1% NaOH. When used (for instance for determining the binding affinities of the antibodies of the present invention), it may be expedient to dilute said solution in an appropriate manner. Further, it is usually expedient to use said solution within 2 hours, in particular within 1 hour, and especially within 30 minutes after its preparation.

More specifically, the term “Aβ(1-40) monomer” here refers to an Aβ(1-40) monomer preparation as described in reference example 1 herein, and the term “Aβ(1-42) monomer” here refers to an Aβ(1-42) preparation as described in reference example 2 herein.

The term “fibril” here refers to a molecular structure that comprises assemblies of non-covalently associated, individual Aβ(X—Y) peptides, which show fibrillary structure in the electron microscope, which bind Congo red and then exhibit birefringence under polarized light and whose X-ray diffraction pattern is a cross-β structure.

In another aspect of the invention, a fibril is a molecular structure obtainable by a process that comprises the self-induced polymeric aggregation of a suitable Aβ peptide in the absence of detergents, e. g. in 0.1 M HCl, leading to the formation of aggregates of more than 24, preferably more than 100 units. This process is well known in the art. Expediently, Aβ(X—Y) fibrils are used in the form of an aqueous solution. In a particularly preferred embodiment of the invention, the aqueous fibril solution is made by dissolving the Aβ peptide in 0.1% NH₄OH, diluting it 1:4 with 20 mM NaH₂PO₄, 140 mM NaCl, pH 7.4, followed by readjusting the pH to 7.4, incubating the solution at 37° C. for 20 h, followed by centrifugation at 10,000 g for 10 min and resuspension in 20 mM NaH₂PO₄, 140 mM NaCl, pH 7.4.

The term “Aβ(X—Y) fibril” here refers to a fibril consisting essentially of Aβ(X—Y) subunits, where it is preferred if on average at least 90% of the subunits are of the Aβ(X—Y) type, more preferred if at least 98% of the subunits are of the Aβ(X—Y) type, and most preferred if the content of non-Aβ(X—Y) peptides is below the detection threshold.

More specifically, the term “Aβ(1-42) fibril” here refers to a Aβ(1-42) fibril preparation as described in reference example 6 herein.

In another aspect, such an immune response is characterized by comprising an antibody having a binding affinity to an immunogenic product of the invention or to an Aβ(20-42) globulomer that is greater than the binding affinity of the antibody to an Aβ(1-42) globulomer or Aβ(12-42) globulomer.

Accordingly, in a further aspect of the invention, the immunogenic product described herein is capable of eliciting a polyclonal antiserum having an affinity to an immunogenic product of the invention or an Aβ(20-42) globulomer which is at least 2 times, e. g. at least 3 times or at least 5 times, preferably at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times greater than the affinity of the antiserum to at least one Aβ globulomer selected from the group consisting of Aβ(1-42) globulomer and Aβ(12-42) globulomer.

The binding affinities of antibodies (monoclonal or polyclonal) to a given antigen (such as the immunogenic products of the present invention) may be evaluated by using standardized in-vitro immunoassays such as ELISA, dot blot or surface plasmon resonance analyses. The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, NJ). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnsson, B., et al. (1991) Anal. Biochem. 198:268-277.

According to a particular embodiment, the affinities defined herein refer to the values obtained by performing a dot blot as described herein and evaluating it by densitometry. According to a particular embodiment of the invention, determining the binding affinity by dot blot comprises the following: a certain amount of the antigen (e.g. the immunogenic product of the invention; Aβ(X—Y) oligomer, Aβ(X—Y) monomer or Aβ(X—Y) fibrils, as defined above) or, expediently, an appropriate dilution thereof, for instance in 20 mM NaH₂PO₄, 140 mM NaCl, pH 7.4, 0.2 mg/ml BSA to an antigen concentration of, for example, 100 pmol/μl, 10 pmol/μl, 1 pmol/μl, 0.1 pmol/μl and 0.01 pmol/μl, is dotted onto a nitrocellulose membrane, the membrane is then blocked with milk to prevent unspecific binding and washed, then contacted with the antibody or antiserum of interest followed by detection of the latter by means of an enzyme-conjugated secondary antibody and a colorimetric reaction; at defined antibody concentrations, the amount of antibody bound allows affinity determination. Thus the relative affinity of two different antibodies or antisera to one antigen, or of one antibody or antiserum to two different antigens, is defined herein as the relation of the respective amounts of antigen-bound antibody observed with the two antibody/antiserum-antigen combinations under otherwise identical dot blot conditions. Unlike a similar approach based on Western blotting, the dot blot approach will determine an antibody's affinity to a given antigen in the latter's natural conformation; unlike the ELISA approach, the dot blot approach does not suffer from differences in the affinities between different targets and the matrix, thereby allowing for more precise comparisons between different antigens.

The term “greater affinity” here refers to a degree of interaction where the equilibrium between unbound antibody and unbound immunogenic product or globulomer on the one hand and antibody-immunogenic product/globulomer complex on the other is further in favor of the complex. Likewise, the term “smaller affinity” here refers to a degree of interaction where the equilibrium between unbound antibody and unbound immunogenic product or globulomer on the one hand and antibody-immunogenic product/globulomer complex on the other is further in favor of the unbound antibody and unbound immunogenic product/globulomer. The term “greater affinity” is synonymous with the term “higher affinity” and term “smaller affinity” is synonymous with the term “lower affinity”.

The term “K_{D}” (also “K_{d}” or “K_{D}”), as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant (kₒₙ), the dissociation rate constant (k_{off}), and the equilibrium dissociation constant (K_{D}) are used to represent the binding affinity of a binding protein (e.g., an antibody) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Idaho) can also be used.

According to a particular embodiment, the immunogenic products of the invention are soluble, in particular soluble in aqueous media (e.g. an aqueous solution of 5 mM NaH₂PO₄ and 35 mM NaCl, more specifically an aqueous solution of 5 mM NaH₂PO₄ and 35 mM NaCl comprising an amphipatic agent in a concentration as indicated below or an aqueous solution of 5 mM NaH₂PO₄ and 35 mM NaCl having a pH of 8 to 10, 8.0 to 9.5, or 8.0 to 9.0). Solubilities of at least 0.1, 1, or 5 mg protein per mL solution are expedient. Solubility can be checked by centrifugation. An immunogenic product is soluble if it does not precipitate upon centrifugation at 10,000×g and a temperature in the range of 10 to 40° C., e.g. at 37° C.

Further, it is preferred that the immunogenic product of the invention comprises a plurality, e.g. 2 to 28, of the Aβ amino acid sequences described herein.

Thus, the immunogenic product of the invention is, in particular, an oligomer of Aβ muteins, that may optionally be truncated and/or crosslinked.

The terms “Aβ oligomer” or “Aβ mutein oligomer” as used herein refer to a soluble, (in the absence of deliberate crosslinking) non-covalent association of Aβ polypeptides and Aβ muteins as defined above. According to one aspect, Aβ oligomers are stable, non-fibrillary assemblies of Aβ (mutein) polypeptides which are obtainable by incubation with anionic detergents. The term “Aβ globulomer” as used herein refers to an Aβ oligomer having a 3-dimensional globular type structure (“molten globule”, see Barghorn et al., J Neurochem 95: 834-847, 2005). Aβ (mutein) oligomers may be characterized by one or more of the following features:
at least partial cleavability of N-terminal amino acids X-24 with promiscuous proteases (such as thermolysin or endoproteinase GluC) yielding truncated forms of Aβ (mutein) oligomers;
non-accessibility of C-terminal amino acids 25-Y with promiscuous proteases and antibodies;
truncated forms of these oligomers maintain the 3-dimensional core structure of said oligomers with a better accessibility of the core epitope Aβ(18-33).

The term “truncated Aβ oligomer” or “truncated Aβ mutein oligomer” as used herein refers to a truncated form of Aβ (mutein) oligomer which can be obtained by subjecting Aβ oligomer to limited proteolytic digestion. More specifically, truncated Aβ(X—Y) (mutein) oligomers include N-terminally truncated forms wherein X is selected from the group consisting of the numbers 2 . . . 24, and Y is as defined herein, which are obtainable by truncating Aβ(1-Y) (mutein) oligomers by treatment with appropriate proteases. For instance, an Aβ(20-42) oligomer can be obtained by subjecting an Aβ(1-42) oligomer to thermolysin proteolysis, and an Aβ(12-42) oligomer can be obtained by subjecting an Aβ(1-42) oligomer to endoproteinase GluC proteolysis. When the desired degree of proteolysis is reached, the protease is inactivated in a generally known manner. The resulting oligomers may then be isolated following the procedures already described herein and, if required, processed further by further work-up and purification steps.

The oligomers of the invention are obtainable by oligomerization of the corresponding Aβ mutein peptide comprising the Aβ amino acid sequence. The oligomerization comprises a noncovalent aggregation of monomeric Aβ mutein peptide so that the oligomers of the invention can be assumed to be composed of a plurality of Aβ mutein peptides.

The starting material, i.e. Aβ mutein peptide, may be prepared by known peptide-synthetic methods or recombinantly. In addition, a number of these proteins are commercially available. In a particular embodiment, the Aβ mutein peptide is synthetic Aβ mutein peptide.

Said peptide may be produced by chemical synthesis using various solid-phase techniques such as those described in G. Barany and R. B. Merrifield, “The Peptides: Analysis, Synthesis, Biology”; Volume 2-“Special Methods in Peptide Synthesis, Part A”, pp. 3-284, E. Gross and J. Meienhofer, Eds., Academic Press, New York, 1980; and in J. M. Stewart and J. D. Young, “Solid-Phase Peptide Synthesis”, 2nd Ed., Pierce Chemical Co., Rockford, I L, 1984. This strategy is based on the Fmoc (9-Fluorenylmethyl methyl-oxycarbonyl) group for temporary protection of the α-amino group, in combination with the tert-butyl group for temporary protection of the amino acid side chains (see for example E. Atherton and R. C. Sheppard, “The Fluorenylmethoxycarbonyl Amino Protecting Group”, in “The Peptides: Analysis, Synthesis, Biology”; Volume 9-“Special Methods in Peptide Synthesis, Part C”, pp. 1-38, S. Undenfriend and J. Meienhofer, Eds., Academic Press, San Diego, 1987.

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively. Alternatively, in cases where a C-terminal amino alcohol is present, the C-terminal residue may be attached to 2-Methoxy-4-alkoxybenzyl alcohol resin (SASRIN™, Bachem Bioscience, Inc., King of Prussia, PA) as described herein and, after completion of the peptide sequence assembly, the resulting peptide alcohol is released with LiBH₄ in THF (see J. M. Stewart and J. D. Young, supra, p. 92).

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valeryl-aminomethyl-Merrifield resin (PAL resin), for C-terminal carboxamides. Coupling of first and subsequent amino acids can be accomplished using HOBT or HOAT active esters produced from DIC/HOBT, HBTU/HOBT, BOP, PyBOP, or from DIC/HOAT, HATU/HOAT, respectively. Preferred solid supports are: 2-Chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to facilitate dissolution of the amino acid.

The syntheses can be carried out by using a peptide synthesizer, such as an Advanced Chemtech Multiple Peptide Synthesizer (MPS396) or an Applied Biosystems Inc. peptide synthesizer (ABI 433a).

Alternatively, any other appropriate methodology known to those familiar with the art could be used, including: 1) synthesis of multiple copies of the desired peptide separated by the appropriate cleavage sites for enzymatic or chemical cleavage of peptide bonds, resulting in the desired peptide, 2) recombinant expression of APP in any system known to those familiar with the art, and containing the amino acid sequence, followed by either enzymatic or chemical processing to yield the desired peptide, 3) recombinant expression of the desired peptide as a fusion protein in any system known to those familiar with the art, 4) recombinant expression of the desired peptide directly in any system known to those familiar with the art.

The recombinant expression of amyloid β peptides is described in WO2007/064917. Moreover, general methods for expression of heterologous proteins in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91: 501; Chaiken 1. M. (1981) CRC Crit. Rev. Biochem. 11: 255; Kaiser et al. (1989) Science 243: 187; Merrifield, B. (1986) Science 232: 342; Kent, S. B. H. (1988) Ann. Rev. Biochem. 57: 957; and Offord, R. E. (1980) Semisynthetic Proteins, Wiley Publishing).

The peptide obtained is then subjected to conditions that allow the oligomer to form. Conditions suitable for oligomer formation are described in, for instance, WO 2004/067561; WO 2006/094724; S. Barghorn et al., J. Neurochem. 95, 834 (2005) and WO 2007/064917, which are incorporated herein by reference.

In a first step, monomeric Aβ mutein peptide is dissolved in a solvent. Preferably, the solvent is a hydrogen bond-breaking agent. The purpose of this treatment is to provide a solution of the unfolded peptide.

Suitable hydrogen bond-breaking agents are known in the art. These include organic compounds such as 1,1,1,3,3,3-hexafluoro-2-propanol (HFIP) and aqueous solutions of bases such as sodium hydroxide, potassium hydroxide, formic acid, 2,2,2-trifluoroethanol (TFE), urea and guanidinium chloride.

According to a particular embodiment, the hydrogen bond-breaking agent is HFIP.

In order to help the Aβ mutein peptide dissolving in the hydrogen bond-breaking agent the mixture may be subjected to agitation, e.g. shaking. Times of dissolution of a few minutes to a few hours, for example 15 minutes to 5 hours, are sufficient when the temperature is from 22 to 50° C. For instance, the peptide can be expediently dissolved by shaking it in HFIP for about 2.5 hours at about 37° C.

The amount of Aβ mutein peptide may be such that 2 mg/mL to 50 mg/mL, 5 mg/mL to 40 mg/mL, or 5 mg/mL to 30 mg/mL of peptide are dissolved in the hydrogen bond-breaking agent. For instance, the concentration of Aβ mutein peptide dissolved in the hydrogen bond-breaking agent can be expediently adjusted to about 6 mg/mL in HFIP. It is expedient if a clear solution is obtained by dissolving the Aβ mutein peptide in the hydrogen bond-breaking agent.

The hydrogen bond-breaking agent is then removed, e.g. by evaporation, and the residue is resuspended in a suitable solvent, e.g. DMSO. The amount of Aβ mutein peptide may be such that 1 mM to 10 mM, 2 mM to 8 mM, or 4 mM to 6 mM of peptide are resuspended in the solvent. For instance, the concentration of resuspended Aβ mutein peptide can be expediently adjusted to about 5 mM in DMSO.

In a further step, an amphipatic agent is added to the solution of Aβ mutein peptide in the hydrogen bond-breaking agent. The addition of the amphipatic agent induces the oligomerization of the peptide to give the oligomers.

Amphipathic agents include fatty acids or detergents, some of which are listed in WO2007064917, which are incorporated herein by reference.

For instance, sulfates, in particular alkyl sulfates and alkyl ether sulfates; sulfonates, e.g. sodium dodecyl sulphate (SDS), carboxylic acids, such as fatty acids, e.g. lauric acid, sarcosines, e.g. N-lauroylsarcosin (also known as sarkosyl NL-30 or Gardol®), alkylaryl alcohol polyoxyethylene ethers, such as octylphenol polyoxyethylene ethers, e.g. tert-octylphenol x 9-10EO (also known as Triton® X100), or alkylaryl nonylphenol polyoxyethylene ethers, e.g. nonylphenol x 20EO (also known as Tergitol® NP-40), 3-(3-cholamidopropyldimethylammonio-1-propane sulfonate (CHAPS), dodecyl-N,N-dimethyl-3-amino-1-propane sulfonate (DDAP), and amines, in particular alkyl amines, e.g. dodecylamine, can be expediently used as amphipatic agent in the process of the invention. Also, sugar surfactants, in particular polyethoxylated sorbitol esters, such as, for example, polyethoxylated sorbitol fatty acid esters, e.g. polyoxyethylene sorbitan monooleate (also known as Polysorbat 80 or Tween® 80), can be expediently used as amphipatic agent in the process of the invention.

According to a particular embodiment, the amphipatic agent is added in the form of an aqueous solution comprising the amphipatic agent. Said solution may be buffered. A pH value in the range from 6.0 to 10.0, 6.5 to 9.5, or 7.0 to 9.0 proves expedient. For instance, a buffered aqueous solution having a pH value of about 7.4 can expediently be used. Suitable buffered aqueous solutions are known in the art. For instance, an aqueous solution comprising 5 mM NaH₂PO₄ and 35 mM NaCl can expediently be used. By adding the aqueous solution to the Aβ mutein peptide is diluted. An amount of aqueous solution added in the range of 5 to 50, 7 to 30, or 8 to 25 times the volume of the resuspended Aβ mutein peptide proves expedient. For instance, the amount of aqueous solution to be added can expediently be about 10 times the volume of the resuspended Aβ mutein peptide.

The concentration of amphipatic agent to be chosen depends on the agent used. If SDS is used, a concentration in the range from 0.05 to 0.7% by weight, from 0.075 to 0.4% by weight, or from 0.1 to 0.3% by weight in the incubation mixture proves expedient. For instance, a buffered aqueous solution comprising about 0.2% by weight of SDS can expediently be used. If lauric acid or N-lauroylsarcosin is used, somewhat higher concentrations are expedient, for example in a range from 0.1 to 1.0% by weight, from 0.25 to 0.75% by weight, or from 0.4 to 0.6% by weight. For instance, a buffered aqueous solution comprising about 0.5% by weight of lauric acid or N-lauroylsarcosin can expediently be used. If polyoxyethylene sorbitan monooleate (e.g. Tween® 80) is used, a concentration in the range from 0.05 to 1% by weight, from 0.075 to 0.5% by weight, or from 0.1 to 0.3% by weight in the incubation mixture proves expedient.

Usually, the resuspended Aβ mutein peptide and the buffered aqueous solution are mixed, expediently under agitation, e.g. vortexing.

Before the mixture is incubated to complete oligomer formation, it may be expedient to remove solids from the mixture.

Times of incubation for oligomer formation may range from a few minutes to a few hours. 1 hour to 48 hours, 2 hours to 36 hours, or 5 hours to 24 hours are sufficient when the temperature of incubation is from 15 to 50° C., from 18 to 45° C. or from 20 to 40° C. For instance, oligomer formation is complete if the mixture is incubated for about 24 hours at about 37° C.

Expediently, the incubation is carried out in two steps, i.e. after a first period of incubation the preparation is diluted (e.g. with water) followed by a second period of incubation.

Times of incubation in the first period may range from a few minutes to a few hours. 1 hour to 24 hours, 2 hours to 12 hours, or 4 hours to 8 hours are sufficient when the temperature of incubation is from 15 to 50° C., from 18 to 45° C. or from 20 to 40° C. For instance, the mixture is incubated for about 6 hours at about 37° C.

Dilution of the incubation mixture can be effected in manner known per se. According to a particular embodiment, dilution comprises adding water. Expediently, the incubation mixture is diluted about 2-fold to 20-fold, 3-fold to 15-fold, or 4-fold to 10-fold, e.g. 4-fold (1:3).

Times of incubation in the second period for completing oligomer formation may range from a few minutes to a few hours. 1 hour to 36 hours, 2 hours to 24 hours, or 4 hours to 18 hours are sufficient when the temperature of incubation is from 15 to 50° C., from 18 to 45° C. or from 20 to 40° C. For instance, oligomer formation is complete if the mixture is incubated for about 18 hours at about 37° C.

Once oligomer formation is complete, it may be expedient to centrifuge the incubation mixture and obtain the supernatant of centrifuged incubation mixture. For instance, centrifugation for about 20 minutes at about 3,000×g proves expedient.

According to a particular embodiment, the supernatant of centrifuged incubation mixture can then be frozen. For instance, the supernatant of centrifuged incubation mixture can expediently be frozen at −30° C. for 30 minutes. The frozen supernatant may then be thawed and optionally the thawed supernatant is again centrifuged (e.g. for 10 minutes at 10,000×g) and the supernatant of centrifuged mixture is obtained.

The oligomer preparation obtainable by this process may be used as such or subjected to further work-up, e.g. in order to concentrate and/or purify the oligomers.

According to a particular embodiment, the process of the invention comprises a step of concentrating the incubation mixture.

Concentrating the incubation mixture can be effected in manner known per se. According to a particular embodiment, concentrating is done by ultracentifugation. Ultracentifugation is a method well-known in the art. Ultracentifugation comprising a 10 to 100, a 20 to 80, or a 25 to 50 kDa cut-off proves expedient. For instance, the oligomers of the invention can expediently be concentrated by ultracentifugation comprising an about 30 kDa cut-off.

Ultracentrifugation reduces the volume of the incubation mixture while maintaining the amount of oligomer that is present in the incubation mixture. Thus, it is expedient to reduce the volume to 1 to 40%, 2 to 35%, or 4 to 33%. For instance, the volume of the incubation mixture can expediently be reduced by ultracentrifugation to about 32%, 10% or 5%.

According to a particular embodiment, the process of the invention comprises the step of reducing the salt concentration of the incubation mixture or the concentrated incubation mixture.

A reduction of the salt concentration (and of the amphipathic agent, the reduction of which is particularly important for a use in active immunization) can be effected in manner known per se. According to a particular embodiment, the salt concentration is reduced by subjecting the incubation mixture or the concentrated incubation mixture to dialysis. Dialysis is a method well-known in the art. For instance, dialysis of the incubation mixture or the concentrated incubation mixture can expediently be performed against a solution comprising 5 mM NaH₂PO₄ and 35 mM NaCl. The solution may also comprise amphipatic agent in a suitable amount. During dialysis it may be expedient to replace the solution by a fresh one.

Dialysis is performed until salt reduction is complete. For instance, about 2.5 hours at about 22° C. prove expedient.

It may further be expedient to centrifuge the dialysate and obtain the supernatant of centrifuged dialysate. For instance, centrifugation for about 10 minutes at about 10,000×g proves expedient.

Thus, according to a particular embodiment, the invention relates to a process for preparing an Aβ mutein oligomer, which process comprises
(i) dissolving monomeric Aβ mutein peptide in a hydrogen bond-breaking agent;
(ii) adding an amphipatic agent, mixing, and incubating;
(iii) diluting and incubating; and
(iv) optionally one or more of the following. centrifuging, reducing salt and/or amphipathic agent concentration by dialysis, concentrating by ultracentrifugation, and
(v) obtaining the supernatant.

According to a particular embodiment, the immunogenic product is a truncated Aβ mutein oligomer.

Such truncated Aβ mutein oligomer are obtainable by a process for preparing Aβ mutein oligomer, which process further comprise the step of (d) proteolytically cleaving the oligomer. Preference is given to endopeptidases, e.g. with an enzyme selected from the group consisting of: trypsin, chymotrypsin, thermolysin, elastase, papain and endoproteinase GluC. Conditions suitable for proteolytically cleaving the oligomer are described in, for instance, WO 2004/067561; WO 2006/094724; and WO 2007/064917, which are incorporated herein by reference. Particular truncated oligomers of the invention are those obtainable by the action of thermolysin.

The immunogenic product of the invention comprises an Aβ amino acid sequence having 62.5% or higher identity to the amino acid sequence Aβ(18-33) set forth in SEQ ID NO:1. Accordingly, the immunogenic product described herein comprises an Aβ amino acid sequence having 62.6% or higher, 68.75% or higher, 75% or higher, 81.25% or higher, 87.5% or higher, or 93.75 or higher identity to the amino acid sequence V₁₈F₁₉F₂₀A₂₁E₂₂D₂₃V₂₄G₂₅S₂₆N₂₇K₂₈G₂₉A₃₀I₃₁I₃₂G₃₃ [SEQ ID NO:2; Aβ(18-33)].

The term “identity” refers to the relatedness of two sequences on a amino acid-by-amino acid basis over a particular comparison window or segment. Thus, identity is defined as the degree of sameness, correspondence or equivalence between two amino acid sequences. “Percentage of sequence identity” is calculated by comparing two optimally aligned sequences over a particular region, determining the number of positions at which the identical amino acid occurs in both sequences in order to yield the number of matched positions, dividing the number of such positions by the total number of positions in the segment being compared and multiplying the result by 100. Optimal alignment of sequences may be conducted by the algorithm of Smith & Waterman, Appl. Math. 2: 482, 1981, by the algorithm of Needleman & Wunsch, J. Mol. Biol. 48: 443, 1970, by the method of Pearson & Lipman, Proc. Natl. Acad. Sci. (USA) 85: 2444, 1988, and by computer programs which implement the relevant algorithms (e.g., Clustal Macaw Pileup (http://cmgm.stanford.edu/biochem218/11Multiple.pdf; Higgins et al., CABIOS. 5L151-153, 1989), FASTDB (Intelligenetics), BLAST (National Center for Biomedical Information; Altschul et al., Nucleic Acids Research 25: 3389-3402, 1997), PILEUP (Genetics Computer Group, Madison, WI) or GAP, BESTFIT, FASTA and TFASTA (Wisconsin Genetics Software Package Release 7.0, Genetics Computer Group, Madison, WI)).

According to one embodiment of the invention, the amino acid sequence comprised by the immunogenic product of the invention is characterized by a particular secondary structure comprising a loop (synonym: turn). A loop (or turn) as used herein is meant to define the close approach (usually <7 Å) of at least two Ca atoms.

Suitable loops include α-, β-, γ-, and π-loops. According to one embodiment of the invention, the loop is a β-loop. A β-loop as used herein is meant to define a loop which is characterized by hydrogen bond(s) in which the donor and acceptor residues are separated by three residues (i→i+/−3H-bonding).

According to a particular embodiment of the invention, the loop is a β-hairpin loop. A β-hairpin loop as used herein is meant to define a loop, in which the direction of the peptide backbone reverses and the flanking secondary structure elements interact.

According to a particular embodiment of the invention, the loop, which may preferably be a β-hairpin loop, comprises a sequence selected from V₂₄G₂₅S₂₆N₂₇ [SEQ ID NO:10; Aβ(24-27)] and D₂₃V₂₄G₂₅S₂₆N₂₇K₂₈ [SEQ ID NO: 11; Aβ(23-28)].

In particular, the amino acid sequence of the immunogenic product described herein forms an intramolecular antiparallel β-sheet. An antiparallel β-sheet as used herein is meant to define an assembly of at least two β-strands connected laterally by three or more hydrogen bonds, forming a generally twisted, pleated sheet. A β-strand is a stretch of amino acids comprising typically 3-10 amino acids whose peptide backbones are almost fully extended.

In a related aspect of the invention, the immunogenic product described herein comprises an amino acid sequence in which the β-strands forming the antiparallel β-sheet are connected via the loop, preferably the β-hairpin loop defined herein.

According to a particular embodiment of said aspect, the amino acid sequence portions of the product corresponding to F₁₉F₂₀A₂₁ [SEQ ID NO:8; Aβ(19-21)] and A₃₀I₃₁I₃₂ [SEQ ID NO:9; Aβ(30-32)] are in anti-parallel orientation.

The oligomers of mutein Aβ peptides may be further characterized by particular interactions between two or more mutein Aβ peptides.

In one aspect of the invention, the immunogenic product described herein comprises an Aβ amino acid sequence having 72% or higher, 77% or higher, 81% or higher, 86% or higher, 90% or higher, or 95% or higher identity to the amino acid sequence identity to the amino acid sequence

[SEQ ID NO: 3; Aβ(18-39)]
V₁₈F₁₉F₂₀A₂₁E₂₂D₂₃V₂₄G₂₅S₂₆N₂₇K₂₈G₂₉A₃₀I₃₁I₃₂G₃₃L₃₄M₃₅V₃₆G₃₇G₃₈V₃₉.

In a related aspect of the invention, said immunogenic product comprises a first amino acid sequence L^{A}₃₄M^{A}₃₅V^{A}₃₆G^{A}₃₇G^{A}₃₈ [SEQ ID NO:5; Aβ(34-38)] that is in parallel orientation to a second amino acid sequence L^{B}₃₄M^{B}₃₅V^{B}₃₆G^{B}₃₇G^{B}₃₈. In this case, the interproton distance for at least one atom pair selected from the group consisting of M^{A}₃₅(NH)—V^{B}₃₆(NH), G^{A}₃₇(NH)-G^{B}₃₈(NH), L^{A}₃₄(NH)-L^{B}₃₄(CγH₃), M^{A}₃₅(NH)—V^{B}₃₆(CγH₃) may be 1.8 to 6.5 Angstroms.

In a further related aspect of the invention, said immunogenic product comprises a first amino acid sequence G^{A}₃₃L^{A}₃₄M^{A}₃₅V^{A}₃₆G^{A}₃₇G^{A}₃₈V^{A}₃₉ [SEQ ID NO:6; Aβ(33-38)] that is in parallel orientation to a second amino acid sequence G^{B}₃₃L^{B}₃₄M^{B}₃₅V^{B}₃₆G^{B}₃₇G^{B}₃₈V^{B}₃₉. In this case, the interproton distance for at least one atom pair selected from the group consisting of G^{A}₃₃(NH)-G^{B}₃₄(NH), M^{A}₃₅(NH)—V^{B}₃₆(NH), G^{A}₃₇(NH)-G^{B}₃₈(NH), L^{A}₃₄(NH)-L^{B}₃₄(CγH₃), M^{A}₃₅(NH)—V^{B}₃₆(CγH₃), G^{A}₃₈(NH)—V^{B}₃₉(CγH₃) and V^{A}₃₉(NH)—V^{B}₃₉(CγH₃) may be 1.8 to 6.5 Angstroms.

In a further related aspect of the invention, said immunogenic product comprises an inter-molecular parallel β-sheet between two Aβ amino acid sequences. In a particular aspect of the invention, said inter-molecular parallel β-sheet comprises a first amino acid sequence G^{A}₃₃L^{A}₃₄M^{A}₃₅V^{A}₃₆G^{A}₃₇G^{A}₃₈V^{A}₃₉ [SEQ ID NO:7; Aβ(33-39)] and a second amino acid sequence G^{B}₃₃L^{B}₃₄M^{B}₃₅V^{B}₃₆G^{B}₃₇G^{B}₃₈V^{B}₃₉. In this cases, the atom pairs G^{A}₃₃(CO)-L^{B}₃₄(N), L^{B}₃₄(CO)-M^{A}₃₅(N), M^{A}₃₅(CO)—V^{B}₃₆(N), V^{B}₃₆(CO)-G^{A}₃₇(N), and G^{B}₃₇(CO)-G^{A}₃₈(N) may be at a distance of 3.3±0.5 Å, wherein CO indicates the backbone oxygen atom, and the phi ((p) angles of the residues range from −180 to −30 and psi (ty) angles of the residues range from approximately 60 to 180 or from approximately −180 to −150.

Interproton distances defining the structure of the antiparallel β-sheet can be determined by the intra-molecular nuclear Overhauser effects (NOEs) between the backbone amides and between the backbone amides and side chains.

Interproton distances defining the structure of the parallel β-sheet can be determined by inter-molecular NOEs between backbone NH—NH and between backbone NH and methyl groups of the side chains.

The intra- vs. inter-molecular NOEs can be distinguished using different isotope-labeled samples, as described, for instance in WO2007/064917, in particular Example V, part G, NMR Features, which is incorporated herein by reference.

Using the NOE-derived distance restraints from the analysis of the NMR data, structures can be calculated, e.g. using the program CNX [A. T. Brunger, et al., Acta Crystallogr. D54 (Pt 5), 905-21, (1998)] by using a simulated annealing protocol [M. Nilges, et al., FEBS Lett. 229, 317-324, (1988)], thereby providing further intra-molecular and/or inter-molecular distances between two atoms.

In one aspect of the invention, the immunogenic product described herein comprises an Aβ amino acid sequence having 62.5% or higher, 64% or higher, 67% or higher, 71% or higher, 75% or higher, 78% or higher, 82% or higher, 85% or higher, 89% or higher, 92% or higher, or 96% or higher identity to a portion (X—Y) of amino acid sequence

[SEQ ID NO: 4; Aβ(12-39)]
V₁₂H₁₃H₁₄Q₁₅K₁₆L₁₇V₁₈F₁₉F₂₀A₂₁E₂₂D₂₃V₂₄G₂₅S₂₆N₂₇K₂₈G₂₉A₃₀I₃₁I₃₂G₃₃L₃₄M₃₅V₃₆G₃₇G₃₈V₃₉,

X being selected from the group consisting of the numbers 12 . . . 18 and Y being selected from the group consisting of the numbers 33 . . . 39.

In a related aspect of the invention, at least 2 non-contiguous residues of said amino acid sequence are covalently linked with each other, e.g. via a direct covalent bond or via a linker. In particular, at least one of the amino acid residues corresponding to V₁₂, H₁₃, H₁₄, Q₁₅, K₁₆, L₁₇, V₁₈, F₁₉, F₂₀, A₂₁ E₂₂ or D₂₃ of [SEQ ID NO:4; Aβ(12-39)] and at least one of the amino acid residues corresponding to K₂₈, G₂₉, A₃₀, I₃₁, I₃₂, G₃₃, L₃₄, M₃₅, V₃₆, G₃₇, G₃₈, V₃₉ of [SEQ ID NO:4; Aβ(12-39)] are covalently linked with each other.

A covalent linkage between two amino acid residues may be established by a variety of means well known in the art, for instance by disulfide bridge formation or cross-linking techniques. In particular, the side chains of amino acid residues may be linked with each other. Especially side chains with a functional group, e.g., a thiol, amino, carboxyl or hydroxyl group, may linked with each other directly, such as two cysteine residues which form a disulfide bridge, or indirectly via a linker. Accordingly, the amino acid residue that is covalently linked to the other amino acid residues may in particular be that of an amino acid residue selected from the group consisting of cysteine, lysine, aspartic acid and glutamic acid.

The crosslinking of proteins has a long and exhaustive history, with large amounts of literature precedent. Any methodology known to those familiar with the art that allows for specific covalent cross-links to be made between natural or non-natural amino acid side chains can be used to form the position specific cross-links envisioned in this invention. Some examples of this methodology are listed below.

There are a large number of chemical cross-linking agents that are known to those skilled in the art. For the present invention, the preferred cross-linking agents include homobifunctional and heterobifunctional cross-linkers, with heterobifunctional cross-linkers being preferred due to their suitability to link amino acids in a stepwise manner.

Also, heterobifunctional cross-linkers provide the ability to establish more specific linkages, thereby reducing the occurrences of unwanted side reactions.

A wide variety of heterobifunctional cross-linkers are known in the art.

These include heterobifunctional cross-linkers for forming linkages between two amino (—NH₂) groups, one amino and one thiol (or sufhydryl, i.e., —SH) group, or two thiol groups.

One reactive group useful as part of a heterobifunctional cross-linker is an amine-reactive group. Common amine-reactive groups include N-hydroxysuccinimide (NHS) esters. NHS esters react specifically with free amines (e.g., lysine residues) in minutes, under slightly acidic to neutral (pH 6.5-7.5) conditions.

It is noted that the cross-linking agents having N-hydroxysuccinimide moieties can also be used in the form of their N-hydroxysulfosuccinimide analogs, which generally have greater water solubility.

Another reactive group useful as part of a heterobifunctional cross-linker is a thiol reactive group. Common thiol reactive groups include maleimides, halogens, and pyridyl disulfides. Maleimides react specifically with free thiol groups (e.g., in cysteine residues) in minutes, preferably under slightly acidic to neutral (pH 6.5-7.5) conditions.

Halogens (iodoacetyl functions) react with —SH groups at physiological pH's. Both of these reactive groups result in the formation of stable thioether bonds.

For instance, succinimidyl-4-(N-maleimidomethyl)-cyclohexane-1-carboxylate (SMCC) or sulfo-SMCC can be used to form a cross-link between the amine of, e.g., a Lys side chain and the free —SH of, e.g., a Cys side chain. The amine-reactive N-hydroxysuccinimide (NHS) ester will react with an amino group (e.g., that of a Lys residue) to form a stable amide bond. The resulting maleimide-activated peptide will then react with a sulfhydryl group of the same peptide (e.g., that of a Cys residue) to form a disulfide bond, thereby establishing the covalent linkage. This chemistry is well described in the literature; see for instance: Uto, I., et al. (1991). J. Immunol. Methods 138, 87-94; Bieniarz, C., et al. (1996). Extended Length Heterobifunctional Coupling Agents for Protein Conjugations. Bioconjug. Chem. 7, 88-95; Chrisey, L. A., et al. (1996). Nucleic Acids Res. 24(15), 3031-3039; Kuijpers, W. H., et al. (1993). Bioconjug. Chem. 4(1), 94-102; Brinkley, M. A. (1992). A survey of methods for preparing protein conjugates with dyes, haptens and crosslinking reagents. Bioconjugate Chem. 3, 2-13; Hashida, S., et al. (1984). More useful maleimide compounds for the conjugation of Fab to horseradish peroxidase through thiol groups in the hinge. J. Appl. Biochem. 6, 56-63; Mattson, G., et al. (1993). A practical approach to crosslinking. Molecular Biology Reports 17, 167-183; Partis, M. D., et al. (1983). Crosslinking of proteins by omega-maleimido alkanoyl N-hydroxysuccinimide esters. J. Protein. Chem. 2, 263-277; Samoszuk, M. K., et al. (1989). A peroxide-generating immunoconjugate directed to eosinophil peroxidase is cytotoxic to Hodgkin's disease cells in vitro. Antibody, Immunoconjugates and Radiopharmaceuticals 2, 37-45; Yoshitake, S., et al. (1982). Mild and efficient conjugation of rabbit Fab and horseradish peroxidase using a maleimide compound and its use for enzyme immunoassay. J. Biochem. 92, 1413-1424.

Further heterobifunctional cross-linkers can be used in a similar fashion, e.g., ([N-ε-maleimidocaproyloxy]succinimide ester, N-[γ-maleimidobutyryloxy]succinimide ester, N-[κ-maleimidoundecanoyloxy]sulfosuccinimide ester, m-maleimidobenzoyl-N-hydroxysuccinimide ester (MBS), or their sulfosuccinimide analogs (e.g. sulfo-MBS).

A further example of a heterobifunctional cross-linker that can be used to form a cross-link between the amine of, e.g., a Lys side chain and the free —SH of, e.g., a Cys side chain, is succinimidyl-6-[(3-(2-pyridyldithio)-propionate]-hexanoate (LC-SPDP) or sulfo-LC-SPDP. The amine-reactive N-hydroxysuccinimide (NHS) ester will react with an amino group (e.g., that of a Lys residue) to form a stable amide bond. The resulting peptide has a pyridyldisulfide group that will then react with a sulfhydryl group of the same peptide (e.g., that of a Cys residue) to form a disulfide bond, thereby establishing the covalent linkage. This chemistry is well described in the literature; see for instance: Carlsson, J., et al. (1978) Biochem. J. 173, 723-737; Stan, R. V. (2004) Am. J. Physiol. Heart Circ. Physiol. 286, H1347-H1353; Mader, C., et al. (2004) J. Bacteriol. 186, 1758-1768.

Further heterobifunctional cross-linkers can be used in a similar fashion, e.g., 4-succinimidyloxycarbonyl-α-methyl-α-(2-pyridyldithio)-toluene (SMPT) or sulfo-SMPT, N-succinimidyl-3-(2-pyridyldithio)-propionate (SPDP) or sulfo-SPDP.

A further example of a heterobifunctional cross-linker that can be used to form a cross-link between the amine of, e.g., a Lys side chain and the free —SH of, e.g., a Cys side chain is N-succinimidyl-S-acetylthioacetate (SATA) or sulfo-SATA. The amine-reactive N-hydroxysuccinimide (NHS) ester will react with an amino group (e.g., that of a Lys residue) to form a stable amide bond. The protected —SH group of the resulting peptide will then be deprotected by treatment with hydroxylamine, and the resulating free —SH will then react with a sulfhydryl group of the same peptide (e.g., that of a Cys residue) to form a disulfide bond, thereby establishing the covalent linkage.

Further heterobifunctional cross-linkers can be used in a similar fashion, e.g., N-succinimidyl-S-acetylthiopropionate or its sulfosuccinimide analog.

Further suitable heterobifunctional cross-linkers include N-succinimidyl-(4-iodoacetyl)-aminobenzoate (SIAB) or sulfo-SIAB.

Specific, stepwise cross-linkages can also be formed between amino (—NH₂) and carboxy (—COOH) groups.

For instance, 1-ethyl-3-(3-dimethylaminopropyl)-carbodiimide hydrochloride (EDC) can be used to form a cross-link between the amine of, e.g., a Lys side chain and the free —COOH of an acidic side chain. The carboxy-reactive carbodiimide will react with an carboxy group (e.g., that of an Asp, Glu, Dab (2,4-diaminobutyric acid), Dap (2,4-diaminopropionic acid), or ornithine residue) to form an unstable o-acylisourea ester.

The reactive o-acylisourea ester will then react with an amino group of the same peptide (e.g., that of a Lys residue) to form an amide bond, thereby establishing the covalent linkage. Alternatively, the reactive o-acylisourea ester may be reacted with N-hydroxysuccinimide, N-hydroxysulfosuccinimide or sulfo-N-hydroxysulfosuccinimide to give the semi-stable amine-reactive NHS ester which will then react with an amino group of the same peptide (e.g., that of a Lys residue) to form an amide bond, thereby establishing the covalent linkage. This chemistry is well described in the literature; see for instance: DeSilva, N. S. (2003) Interactions of Surfactant Protein D with Fatty Acids. Am. J. Respir. Cell Mol. Biol. 29, 757-770; Grabarek, Z. and Gergely, J. (1990) Zero-length crosslinking procedure with the use of active esters. Anal. Biochem. 185, 131-135; Sinz, A. (2003). J. Mass Spectrom. 38, 1225-1237. Staros, J. V., Wright, R. W. and Swingle, D. M. (1986) Enhancement by N-hydroxysulfosuccinimide of water-soluble carbodiimide-mediated coupling reactions. Anal. Biochem. 156, 220-222; Taniuchi, M., et al. (1986). Induction of nerve growth factor receptor in Schwann cells after axotomy. Proc. Natl. Acad. Sci. USA83, 4094-4098.

Heterobifunctional cross-linkers also include the reaction of Lys(N3) and propargyl glycine amino acids. This reaction can be performed in solution or on resin (as described, for instance, in Jiang, S., (2008) Curr. Org. Chem. 12, 1502-1542 and references therein).

A particular class of cross-linkers, in particular heterobifunctional cross-linkers, includes photoreactive cross-linkers.

For instance, (SDA) can be used to form a cross-link between the amine of, e.g., a Lys side chain and the amine of, e.g., another Lys side chain. The amine-reactive N-hydroxysuccinimide (NHS) ester will react with an amino group (e.g., that of a Lys residue) to form a stable amide bond. The resulting peptide has a photo-labile diazirine moiety that, upon exposure to UV light, will react with an amino group (e.g., that of a Lys residue) of the same peptide to form a stable bond, thereby establishing the covalent linkage.

Further suitable photoreactive cross-linkers include bis-[β-(4-azidosalicylamido)-ethyl]-disulfide (BASED) and N-succinimidyl-6-(4′-azido-2′-nitrophenyl-amino)-hexanoate (SANPAH).

In addition to the heterobifunctional cross-linkers, there exist a number of other cross-linking agents including homobifunctional cross-linkers.

These include homobifunctional cross-linkers for forming linkages between two amino (—NH₂) groups.

For instance, disuccinimidyl suberate (DSS) can be used to form a cross-link between the amine of, e.g., a Lys side chain and the amine of, e.g., another Lys side chain. The amine-reactive N-hydroxysuccinimide (NHS) ester will react with an amino group (e.g., that of a Lys residue) to form a stable amide bond. The resulting peptide will then react with another amine group of the same peptide (e.g., that of a Lys residue) to form a further stable amide bond, thereby establishing the covalent linkage.

Further suitable homobifunctional cross-linkers include bismaleimidohexane (BMH) and dimethylpimelimidate (DMP).

Further suitable homobifunctional cross-linkers include a methylenedithioether linkage between two cysteines. The reaction of the peptide with TBAF (tetrabutylammonium fluoride) can be performed on resin containing the partially deprotected peptide followed by cleavage (see, for instance, Ueki et al., (1999) Bioorg. Med. Chem. Lett., 9, 1767-1772, and Ueki et al. in Peptide Science, 1999, 539-541).

Further suitable homobifunctional cross-linking systems include ring closing metathesis reactions between allylglycines (see, for instance, Wels, B. et al., (2005) Bioorg. Med. Chem. 13, 4221-4227) or modified amino acids, e.g. (S)-Fmoc-α(2′ pentenyl)alanine (see, for instance, Walensky, L. D., et al., (2004) Science 305, 1466-1470; Schafmeister, C. E., et al., (2000) J. Am. Chem. Soc. 122, 5891-5892; Qiu, W., et al., (2000) Tetrahedron 56, 2577-2582; Belokon, Y. N., et al., (1998) Tetrahedron: Asymmetry, 9, 4249-4252); Qiu W., (2008) Anaspec poster at 20th American Peptide Society Annual Meeting). These reactions can be performed in solution on the protected peptide fragment or on the resin, respectively.

Homo- and heterobifunctional cross-linker may comprise a spacer arm or bridge. The bridge is the structure that connects the two reactive ends. The most apparent attribute of the bridge is its effect on steric hindrance. In some instances, a longer bridge can more easily span the distance necessary to link two amino acid residues.

While one covalent linkage between 2 non-contiguous amino acid residues may provide sufficient stabilization, the immunogenic products of the invention may comprise more than one covalent linkage.

The conditions that allow the linkage to form will, of course, depend on the type of linkage to form and can easily be determined by the skilled artisan. Reference is made to description of linkages and their chemistry provided herein.

Oligomer and linkage formation can be independently used to ensure that the immunogenic products of the invention have the desired secondary structure. Thus, the present invention provides Aβ mutein oligomers with such linkages and monomeric Aβ mutein peptides having such linkages.

Further, oligomer and linkage formation can be used to ensure that the immunogenic product of the invention has the desired secondary structure. For, instance linkage formation may help to promote proper oligomer formation and vice versa.

In principle, oligomer formation may precede linkage formation. This is advantageous if the pre-formed oligomer directs or promotes the linkages to be formed. Alternatively, linkage formation may precede oligomer formation. This is advantageous if pre-formed linkages direct or promote oligomer formation. Oligomer formation and linkage formation may also take place concomitantly.

Both the Aβ mutein peptide and the oligomer may be prepared using a peptide which differs from the amino acid sequence comprised by the final immunogenic product. For instance, the starting peptide may comprise additional amino acids at its C- and/or N-terminus which will then be removed during the synthesis, e.g., by proteolytic cleavage.

In one embodiment of the invention, oligomers are formed with a peptide and subsequently stabilized by one or more intra-peptide covalent bond(s).

In another embodiment of the invention, oligomers are formed with a peptide, stabilized by one or more intra-peptide covalent bond(s), and subsequently processed by chemical or enzymatic means to a truncated form that better displays the relevant structural elements. Alternatively, oligomers are formed with a peptide, processed by chemical or enzymatic means to a truncated form that better displays relevant structural elements, and subsequently stabilized by one or more intra-peptide covalent bond(s).

In still another embodiment of the invention, a peptide is used to form the relevant structural elements, wherein the peptide would be held in the proper conformation by one or more intra-peptide covalent bond(s), rather than by interaction with adjacent peptides in an oligomer. It is envisioned that these immunogenic products, stabilized with the appropriate intra-peptide covalent bond(s), will present the relevant structural elements as a monomer.

The term “Aβ mutein” as used herein refers to a variant Aβ polypeptide that differs from human amyloid beta (Aβ) protein by one or more one amino acid substitutions. In particular, an Aβ mutein is an Aβ differing from the polypeptide having the amino acid sequence set forth in SEQ ID NO:1 by one, two, three, four, five, six, or more point mutations. Said point mutations are preferably located at hot spots within Aβ(18-33), Aβ(18-25), Aβ(19-24) and most preferably within Aβ(20-22).

Exemplary amino acid substitutions that may be present in Aβ amino acid sequences comprised by the immunogenic products described herein are summarized in Table 1.

TABLE 1
Exemplary amino acid substitutions present in Aβ amino acid
sequences according to the invention. Amino acid substitutions
that are preferred at the respective hot spot are indicated in bold.

| Amino acid substitution | |
|---|---|
| hot spot (indicated as | |
| corresponding amino acid | |
| of SEQ ID NO: 1) | substituted by |
| V18 | H, R, K, D, E, C, N, S, T, Q, P, F, Y, |
| | W |
| F19 | H, R, K, D, E, C, N, S, T, Q, A, G, P, |
| | V, L, M, I |
| F20 | H, R, K, D, E, C, N, S, T, Q, P, A, G, |
| | V, L, M, I |
| A21 | H, R, K, G, P, D, E, F, Y, W |
| E22 | V, L, M, I, F, Y, W, A, C, N, S, T, P, |
| | Q |
| D23 | H, R, K, V, L, M, I, F, Y, W, P, C, N, |
| | S, T, Q |
| V24 | H, R, K, D, E, C, N, S, T, Q, P, F, Y, |
| | W |
| G25 | H, R, K, V, L, M, I, F, Y, W, P, D, E |
| S26 | H, R, K, P, D, E, F, Y, W |
| N27 | H, R, K, V, L, M, I, F, Y, W, P, D, E |
| K28 | D, E, A, G, V, L, M, I, F, Y, W, P, C, |
| | N, S, T, Q |
| G29 | H, R, K, D, E, C, N, S, T, Q, V, L, M, |
| | I, F, Y, W, P |
| A30 | H, R, K, D, E, C, N, S, T, Q, G, V, L, |
| | M, I, F, Y, W, P |
| I31 | H, R, K, D, E, C, N, S, T, Q, P, F, Y, |
| | W |
| I32 | H, R, K, D, E, C, N, S, T, Q, P, F, Y, |
| | W |
| G33 | H, R, K, D, E, C, N, S, T, Q, V, L, M, |
| | I, F, Y, W, P |

In one aspect of the invention, the Aβ amino acid sequence comprised by the immunogenic product described herein is a variant of Aβ(18-33) that differs from the amino acid sequence set forth in SEQ ID NO:2 by one, two, three, four, five or six amino acids being substituted by other amino acids. Said amino acid substitutions may be selected from the point mutations set forth in Table 1. For example, where the Aβ amino acid sequence comprised by the immunogenic product described herein differs from the amino acid sequence of SEQ ID NO:2 by having two amino acid substitutions, one or both of these amino acid substitutions may be selected from the point mutations set forth in Table 1. Said two amino acid substitutions are preferably at hot spots corresponding to amino acid positions E22/G25, F20/E22, F20/131, A21/E22, A21/D23 and E22/S26 of SEQ ID NO:2, in particular at hot spots corresponding to amino acid positions E22/G25 and F20/E22. Where the Aβ amino acid sequence comprised by the immunogenic product described herein differs from the amino acid sequence of SEQ ID NO:2 by having three, four, five or six amino acid substitutions, one, more than one, or all of these amino acid substitutions may be selected from the point mutations set forth in Table 1.

In particular embodiments of the invention, the Aβ amino acid sequence comprised by the immunogenic product described herein differs from the amino acid sequence of SEQ ID NO:2 by having one amino acid substitution selected from E22A and E22V.

In further particular embodiments of the invention, the Aβ amino acid sequence comprised by the immunogenic product described herein differs from the amino acid sequence of SEQ ID NO:2 by having two amino acid substitutions selected from the double mutations F20G/E22A and E22A/G25A.

In a related aspect of the invention, the Aβ amino acid sequence comprised by the immunogenic product described herein differs from the amino acid sequence

(SEQ ID NO: 2; Aβ(18-33)])
V₁₈F₁₉F₂₀A₂₁E₂₂D₂₃V₂₄G₂₅S₂₆N₂₇K₂₈G₂₉A₃₀I₃₁I₃₂G₃₃,

by one, two, three, four, five or six amino acids being substituted by different amino acids. Examples said amino acid sequences comprise amino acid sequences, wherein
the amino acid corresponding to V₁₈ is selected from the group consisting of histidine, arginine, lysine, aspartic acid, glutamic acid, cysteine, asparagine, serine, threonine, glutamine, proline, phenylalanine, tyrosine, and tryptophan;
the amino acid corresponding to F₁₉ is selected from the group consisting of histidine, arginine, lysine, aspartic acid, glutamic acid, cysteine, asparagine, serine, threonine, glutamine, alanine, glycine, proline, valine, leucine, methionine, and isoleucine;
the amino acid corresponding to F₂₀ is selected from the group consisting of histidine, arginine, lysine, aspartic acid, glutamic acid, cysteine, asparagine, serine, threonine, glutamine, proline, alanine, glycine, valine, leucine, methionine, and isoleucine;
the amino acid corresponding to A₂₁ is selected from the group consisting of histidine, arginine, lysine, glycine, proline, aspartic acid, glutamic acid, phenylalanine, tyrosine, and tryptophan;
the amino acid corresponding to E₂₂ is selected from the group consisting of valine, leucine, methionine, isoleucine, phenylalanine, tyrosine, tryptophan, alanine, cysteine, asparagine, serine, threonine, proline, and glutamine;
the amino acid corresponding to D₂₃ is selected from the group consisting of histidine, arginine, lysine, valine, leucine, methionine, isoleucine, phenylalanine, tyrosine, tryptophan, proline, cysteine, asparagine, serine, threonine, and glutamine;
the amino acid corresponding to V₂₄ is selected from the group consisting of histidine, arginine, lysine, aspartic acid, glutamic acid, cysteine, asparagine, serine, threonine, glutamine, proline, phenylalanine, tyrosine, and tryptophan;
the amino acid corresponding to G₂₅ is selected from the group consisting of histidine, arginine, lysine, valine, leucine, methionine, isoleucine, phenylalanine, tyrosine, tryptophan, proline, aspartic acid, and glutamic acid;
the amino acid corresponding to S₂₆ is selected from the group consisting of histidine, arginine, lysine, proline, aspartic acid, glutamic acid, phenylalanine, tyrosine, tryptophan;
the amino acid corresponding to N₂₇ is selected from the group consisting of histidine, arginine, lysine, valine, leucine, methionine, isoleucine, phenylalanine, tyrosine, tryptophan, proline, aspartic acid, and glutamic acid;
the amino acid corresponding to K₂₈ is selected from the group consisting of aspartic acid, glutamic acid, alanine, glycine, valine, leucine, methionine, isoleucine, phenylalanine, tyrosine, tryptophan, proline, cysteine, asparagine, serine, threonine, and glutamine;
the amino acid corresponding to G₂₉ is selected from the group consisting of histidine, arginine, lysine, aspartic acid, glutamic acid, cysteine, asparagine, serine, threonine, glutamine, valine, leucine, methionine, isoleucine, phenylalanine, tyrosine, tryptophan, and proline;
the amino acid corresponding to A₃₀ is selected from the group consisting of histidine, arginine, lysine, aspartic acid, glutamic acid, cysteine, asparagine, serine, threonine, glutamine, glycine, valine, leucine, methionine, isoleucine, phenylalanine, tyrosine, tryptophan, and proline;
the amino acid corresponding to I₃₁ is selected from the group consisting of histidine, arginine, lysine, aspartic acid, glutamic acid, cysteine, asparagine, serine, threonine, glutamine, proline, phenylalanine, tyrosine, and tryptophan;
the amino acid corresponding to I₃₂ is selected from the group consisting of histidine, arginine, lysine, aspartic acid, glutamic acid, cysteine, asparagine, serine, threonine, glutamine, proline, phenylalanine, tyrosine, and tryptophan;
the amino acid corresponding to G₃₃ is selected from the group consisting of histidine, arginine, lysine, aspartic acid, glutamic acid, cysteine, asparagine, serine, threonine, glutamine, valine, leucine, methionine, isoleucine, phenylalanine, tyrosine, tryptophan, and proline;
the amino acid corresponding to F₂₀ is selected from the group consisting of histidine, arginine, lysine, aspartic acid, glutamic acid, cysteine, asparagine, serine, threonine, glutamine, proline, alanine, glycine, valine, leucine, methionine, and isoleucine; and the amino acid corresponding to E₂₂ is selected from the group consisting of alanine, valine, proline, phenylalanine, methionine, isoleucine, tryptophan, cysteine, asparagine, serine, threonine, tyrosine, and leucine;
the amino acid corresponding to F₂₀ is glycine, and the amino acid corresponding to E₂₂ is alanine;
the amino acid corresponding to F₂₀ is selected from the group consisting of histidine, arginine, lysine, aspartic acid, glutamic acid, cysteine, asparagine, serine, threonine, glutamine, proline, alanine, glycine, valine, leucine, methionine, and isoleucine; and
the amino acid corresponding to I₃₁ is selected from the group consisting of histidine, arginine, lysine, aspartic acid, glutamic acid, cysteine, asparagine, serine, threonine, glutamine, proline, phenylalanine, tyrosine, and tryptophan;
the amino acid corresponding to A₂₁ is selected from the group consisting of histidine, arginine, lysine, glycine, proline, aspartic acid, glutamic acid, phenylalanine, tyrosine, and tryptophan; and the amino acid corresponding to E₂₂ is selected from the group consisting of alanine, valine, proline, phenylalanine, methionine, isoleucine, tryptophan, cysteine, asparagine, serine, threonine, tyrosine, and leucine;
the amino acid corresponding to A₂₁ is selected from the group consisting of histidine, arginine, lysine, glycine, proline, aspartic acid, glutamic acid, phenylalanine, tyrosine, and tryptophan; and the amino acid corresponding to D₂₃ is selected from the group consisting of histidine, arginine, lysine, valine, leucine, methionine, isoleucine, phenylalanine, tyrosine, tryptophan, proline, cysteine, asparagine, serine, threonine, and glutamine;
the amino acid corresponding to E₂₂ is selected from the group consisting of valine, leucine, methionine, isoleucine, phenylalanine, tyrosine, tryptophan, alanine, cysteine, asparagine, serine, threonine, proline, and glutamine; and the amino acid corresponding to G₂₅ is selected from the group consisting of histidine, arginine, lysine, valine, leucine, methionine, isoleucine, phenylalanine, tyrosine, tryptophan, proline, aspartic acid, and glutamic acid; or
the amino acid corresponding to E₂₂ is selected from the group consisting of valine, leucine, methionine, isoleucine, phenylalanine, tyrosine, tryptophan, alanine, cysteine, asparagine, serine, threonine, proline, and glutamine; and the amino acid corresponding to S₂₆ is selected from the group consisting of histidine, arginine, lysine, proline, aspartic acid, glutamic acid, phenylalanine, tyrosine, tryptophan.

More specifically, the immunogenic products of the invention comprise an amyloid β (Aβ) amino acid sequence which is identical to a portion (X—Y) of an amino acid sequence selected from the group consisting of:

[SEQ ID NO: 13; Aβ(1-43)F19A]
D₁A₂E₃F₄R₅H₈D₇S₈G₉Y₁₀E₁₁V₁₂H₁₃H₁₄Q₁₅K₁₈L₁₇V₁₈A₁₉F₂₀A₂₁E₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 14; Aβ(1-43)F20A]
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 15; Aβ(1-43)E22A]
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 16; Aβ(1-43)E22F]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁F₂₂D₂₃V₂₄G₂₅S₂₆N₂₇K₂₈
G₂₉A₃₀I₃₁I₃₂G₃₄L₃₄M₃₅V₃₆G₃₇G₃₈V₃₉V₄₀I₄₁A₄₂T₄₃;
[SEQ ID NO: 17; Aβ(1-43)E22V]
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁V₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 18; Aβ(1-43)E22L]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 19; Aβ(1-43)D23K]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂K₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 20; Aβ(1-43)D23L]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂L₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 21; Aβ(1-43)G25V]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄V₂₅S₂₆N₂₇K₂₈
G₂₉A₃₀I₃₁I₃₂G₃₃L₃₄M₃₅V₃₆G₃₇G₃₈V₃₉V₄₀₁₄₁A₄₂T₄₃;
[SEQ ID NO: 22; Aβ(1-43)A30G]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄G₂₅S₂₆N₂₇K₂₈
G₂₉G₃₀I₃₁I₃₂G₃₃L₃₄M₃₅V₃₆G₃₇G₃₈V₃₉V₄₀I₄₁A₄₂T₄₃;
[SEQ ID NO: 23; Aβ(1-43)F20G E22A]
D₁A₂E₃F₄R₅H₆D₇S₈G₉Y₁₀E₁₁V₁₂H₁₃H₁₄Q₁₅K₁₆L₁₇V₁₈F₁₉G₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 24; Aβ(1-43)F20A I31A]
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀A₃₁I₃₂G₃₃L₃₄M₃₅V₃₆G₃₇G₃₈V₃₉V₄₀I₄₁A₄₂T₄₃;
[SEQ ID NO: 25; Aβ(1-43)F20C I31C]
D₁A₂E₃F₄R₅H₆D₇S₈G₉Y₁₀E₁₁V₁₂H₁₃H₁₄Q₁₅K₁₆L₁₇V₁₈F₁₉C₂₀A₂₁E₂₂D₂₃V₂₄G₂₅S₂₆N₂₇K₂₈
G₂₉A₃₀C₃₁I₃₂G₃₃L₃₄M₃₅V₃₆G₃₇G₃₈V₃₉V₄₀I₄₁A₄₂T₄₃;
[SEQ ID NO: 26; Aβ(1-43)A21Q E22L]
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 27; Aβ(1-43)A21L E22Q]
D₁A₂E₃F₄R₅H₆D₇S₈G₉Y₁₀E₁₁V₁₂H₁₃H₁₄Q₁₅K₁₆L₁₇V₁₈F₁₉F₂₀L₂₁Q₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 28; Aβ(1-43)A21Q D23N]
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁E₂₂N₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 29; Aβ(1-43)E22A G25A]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄A₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
and
[SEQ ID NO: 30; Aβ(1-43)E22A S26A]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄G₂₅A₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,

wherein X is selected from the group consisting of the numbers 1 . . . 18, 4 . . . 18, 12 . . . 18 or 18 and Y is selected from the group consisting of the numbers 33 . . . 43, 33 . . . 42, 33 . . . 41, or 33 . . . 40. In particular embodiment, (X—Y) is selected from the group consisting of (1-42), (4-42), (12-42), or (18-42).

The immunogenic products of the invention are, in particular, oligomers which comprise a plurality of a particular amyloid β (Aβ) amino acid sequence as defined above.

The present invention also relates to purified immunogenic products of the invention. According to one embodiment of the present invention, a purified immunogenic product is one which has a purity of more than 80% by weight of total Aβ peptide, preferably of more than 90% by weight of total Aβ peptide, preferably of more than 95% by weight of total Aβ peptide.

It may be expedient that the immunogenic products of the invention comprise, in addition to the amyloid β-derived amino acid sequence, one or more further moieties. For instance, diagnostic applications may require labelling the immunogenic products. Also, in active immunization it may be of advantage to attach moieties which prove expedient in active immunization applications.

Thus, the present invention also relates to immunogenic products, as defined herein, which comprise a covalently linked group that facilitates detection, preferably a fluorophore, e. g. fluorescein isothiocyanate, phycoerythrin, Alexa-488, Aequorea victoria fluorescent protein, Dictyosoma fluorescent protein or any combination or fluorescence-active derivative thereof; a chromophore; a chemoluminophore, e. g. luciferase, preferably Photinus pyralis luciferase, Vibrio fischeri luciferase, or any combination or chemoluminescence-active derivative thereof; an enzymatically active group, e. g. peroxidase, e. g. horseradish peroxidase, or any enzymatically active derivative thereof; an electron-dense group, e. g. a heavy metal containing group, e.g. a gold containing group; a hapten, e. g. a phenol derived hapten; a strongly antigenic structure, e. g. peptide sequence predicted to be antigenic, e. g. predicted to be antigenic by the algorithm of Kolaskar and Tongaonkar; a molecule which helps elicit an immune response to the immunogenic products, e.g., serum albumin, ovalbumin, keyhole limpet hemocyanin, thyroglobulin, a toxoid from bacteria such as tetanus toxoid and diphtheria toxoid, a naturally occurring T cell epitope, a naturally occurring T helper cell epitope; an artificial T-cell epitope such as the pan DR epitope (“PADRE”; WO 95/07707), or another immunostimulatory agent, e.g., mannan, tripalmitoyl-S-glycerine cysteine, and the like; an aptamer for another molecule; a chelating group, e. g. hexahistidinyl; a natural or nature-derived protein structure mediating further specific protein-protein interactions, e. g. a member of the fos/jun pair; a magnetic group, e. g. a ferromagnetic group; or a radioactive group, e. g. a group comprising 1H, 14C, 32P, 35S or 125I or any combination thereof. With a view to avoiding the unfavored pro-inflammatory immune response Th1-pathway, immunogenic products comprising a molecule which is capable of directing the immune response to the anti-inflammatory pathway (Th2-pathway), e.g. molecules comprising a B cell epitope such as PADRE are expected to provide particular advantages in active immunization (see also, Petrushina I., et al., The Journal of Neuroscience 2007, 27(46): 12721-12731; Woodhouse A., et al., Drugs Aging 2007; 24(2): 107-119).

Such groups and methods for linking them to the immunogenic products are known in the art.

The immunogenic products of the invention have many utilities. For instance, they can be used in: 1) immunization-based interventional therapies (e.g., the immunogenic products may be used in active immunization to treat or prevent an amyloidosis); 2) diagnostic testing (e.g., the immunogenic products may be used to diagnose an amyloidosis; 3) providing agents such as antibodies and aptamers that bind to the immunogenic products; and 4) crystallographic or NMR-based structure-based design research for developing agents such as antibodies and aptamers that bind to the immunogenic products.

In active immunization, Aβ(20-42) globulomer was shown to be effective in reversing cognitive defects in Alzheimer Disease transgenic mice. The immunogenic products of the present invention are able to elicit an immune response whose profile is similar to the profile of the immune response elicited by Aβ(20-42) globulomer.

Thus, the invention also relates to the immunogenic products as defined herein for therapeutic uses.

In one aspect, the invention relates to a composition comprising an immunogenic product as disclosed herein, in particular a composition that is a vaccine, i.e. can be used for active immunization. According to a particular embodiment, said compositions are pharmaceutical compositions which further comprise a pharmaceutical acceptable carrier. The composition may further comprise a pharmaceutical acceptable adjuvant such as Complete Freund's Adjuvant (CFA) or an adjuvant comprising an aluminium salt.

The present invention also relates to a method of treating or preventing an amyloidosis in a subject in need thereof, which comprises administering an effective amount of immunogenic product as disclosed herein to the subject. Preferably, the product is for active immunization.

In a related aspect, the present invention relates to an immunogenic product as disclosed herein for use in treating or preventing an amyloidosis, and in particular for active immunization.

The term “amyloidosis” here denotes a number of disorders characterized by abnormal folding, clumping, aggregation and/or accumulation of particular proteins (amyloids, fibrous proteins and their precursors) in various tissues of the body. In Alzheimer's disease and Down's syndrome, nerve tissue is affected, and in cerebral amyloid angiopathy (CAA) blood vessels are affected. According to a particular embodiment of the present invention, an amyloidosis is selected from the group consisting of Alzheimer's disease (AD) and the amyloidosis of Down's syndrome.

In the context of active immunization, it is particularly preferred if the immunogenic product is not able to enter the patient's CNS in significant amounts.

It is also particularly preferred if the pharmaceutical composition comprising the immunogenic product is capable of inducing a strong immune response against Aβ oligomers, preferably a strong immune response directed against Aβ oligomers only, more preferably a strong non-inflammatory antibody-based immune response against Aβ oligomers only. Thus, in one embodiment of the invention the pharmaceutical composition comprises an immunological adjuvant, preferably an adjuvant and a signalling molecule, e. g. a cytokine, that directs the immune response towards the non-inflammatory, antibody-based type. Such adjuvants and signalling molecules are well known to those skilled in the art.

It is particularly preferred if the pharmaceutical composition for active immunization is administered via a route selected from the group consisting of the intravenous route, the intramuscular route, the subcutaneous route, the intranasal route, and by inhalation. It is also particularly preferred if the composition is administered by a method selected from injection, bolus infusion and continuous infusion, each of which may be performed once, repeatedly or in regular intervals.

In a particular embodiment of the invention, long-term continuous infusion is achieved by employing an implantable device. In a further particular embodiment of the invention, the composition is applied as an implantable sustained release or controlled release depot formulation. Suitable formulations and devices are known to those skilled in the art. The details of the method to be used for any given route will depend on the stage and severity of the disease and the overall medical parameters of the subject and are preferably decided upon individually at the treating physician's or veterinary's discretion.

In an especially preferred embodiment of the invention, the pharmaceutical composition for active immunization comprises one or more substances selected from the group consisting of pharmaceutically acceptable preservatives, pharmaceutically acceptable colorants, pharmaceutically acceptable protective colloids, pharmaceutically acceptable pH regulators and pharmaceutically acceptable osmotic pressure regulators. Such substances are described in the art.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

In line with globulomer hypothesis, it is believed that subjects suffering from an amyloidosis develop an immune response against endogenous globulomer epitopes. As the immunogenic products of the present invention react with antibodies that are specifically reactive with said epitopes the oligomers are believed to display the same or a very similar epitope.

The invention thus also relates to the immunogenic products as defined herein for diagnostic uses.

In one aspect, the present invention relates to a method of diagnosing an amyloidosis which comprises providing a sample from the subject suspected of having the amyloidosis, contacting the sample with an immunogenic product as disclosed herein for a time and under conditions sufficient for the formation of a complex comprising the product and an antibody, the presence of the complex indicating the subject has the amyloidosis. According to a particular embodiment, at least the step of contacting the sample is carried out ex vivo and in particular in vitro.

In a related aspect, the present invention relates to an immunogenic product as disclosed herein for use in diagnosing an amyloidosis.

Thus, the immunogenic products of the present invention may be used in a variety of diagnostic methods and assays.

According to one embodiment, the method of diagnosing an amyloidosis in a patient suspected of having this disease comprises the steps of: a) isolating a biological sample from the patient; b) contacting the biological sample with an immunogenic product of the invention for a time and under conditions sufficient for the formation of antibody/product complexes; c) adding a conjugate to the resulting antibody/product complexes for a time and under conditions sufficient to allow the conjugate to bind to the bound antibody, wherein the conjugate comprises an antibody attached to a signal generating compound capable of generating a detectable signal; and d) detecting the presence of antibodies which may be present in the biological sample by detecting a signal generated by the signal generating compound, the signal indicating a diagnosis of an amyloidosis in the patient. According to a particular embodiment, at least one of steps b), c) and d) is carried out ex vivo and in particular in vitro. According to a further particular embodiment, the method does not comprise step a).

According to a further embodiment, the method of diagnosing an amyloidosis in a patient suspected of having this disease comprises the steps of: a) isolating a biological sample from the patient; b) contacting the biological sample with anti-antibody specific for antibodies in the sample for a time and under conditions sufficient to allow for formation of anti-antibody/antibody complexes; b) adding a conjugate to resulting anti-antibody/antibody complexes for a time and under conditions sufficient to allow the conjugate to bind to bound antibody, wherein the conjugate comprises an immunogenic product of the present invention attached to a signal generating compound capable of generating a detectable signal; and c) detecting a signal generated by the signal generating compound, the signal indicating a diagnosis of an amyloidosis in the patient. According to a particular embodiment, at least one of said steps b) and c) is carried out ex vivo and in particular in vitro. According to a further particular embodiment, the method does not comprise step a).

More specifically, as the immunogenic products of the present invention display the globulomer epitope and the globulomer epitope is believed to be an endogenous antigen which gives rise to an endogenous immune response, diagnosis of amyloidoses can be related to the determination of the presence of auto-antibodies which specifically bind to the immunogenic products of the invention.

The invention thus also relates to the use of the immunogenic products as defined herein for preparing a composition for detecting in a subject auto-antibodies that bind to the immunogenic product. Accordingly, the invention also relates to a method of detecting auto-antibodies in a subject, which method comprises administering to the subject an immunogenic product as defined herein and detecting a complex formed by the antibody and the immunogenic product, the presence of the complex indicating the presence of the auto-antibodies. According to a particular embodiment, at least the step of contacting the sample is carried out ex vivo and in particular in vitro. In a particular embodiment of the invention, the subject is suspected of having any form of amyloidosis, e.g. Alzheimer's disease, and detecting auto-antibodies is for diagnosing the presence or absence of any form of amyloidosis, e.g. Alzheimer's disease, in the subject.

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells, organs, tissues, bone marrow, lymph nodes and spleen.

Suitable samples include in particular biological fluids which may be tested in the methods described herein. These include plasma, whole blood, dried whole blood, serum, cerebrospinal fluid or aqueous or organo-aqueous extracts of tissues and cells.

It is particularly preferred if the subject suspected of having an amyloidosis is a subject having the amyloidosis or having an increased risk of getting the amyloidosis.

According to a particular embodiment of the invention, detecting auto-antibodies as described herein further comprises a pre-treatment of the preparation (sample) which causes dissociation of auto-antibody/antigen complexes. A method comprising such a pre-treatment may therefore be used in order to determine the total amount of auto-antibodies present in the preparation (sample) while a method not comprising said pre-treatment may be used in order to determine the amount of auto-antibodies which can still bind to the antigen. Further, both methods will allow to indirectly determine the amount of complexed auto-antibodies.

Conditions suitable for inducing dissociation of auto-antibody/antigen complexes are known to the skilled person. For instance, treating the preparation (sample) with acid, e.g., using a buffer such that the pH of the resulting preparation (sample) is in the range of 1 to 5, preferably in the range of 2 to 4 and in particular in the range of 2 to 3, may be expedient. Suitable buffers include salts in a physiological concentration, e.g. NaCl and acetic acid. A method for separation of antibody/antigen complexes has been described in WO2005/037209, which is incorporated herein in its entirety.

Briefly, dissociating the antibody from the antigen in the antibody/antigen complex comprises the steps of: contacting the sample containing an antibody/antigen complex with a dissociation buffer; incubating the sample; and optionally concentrating the sample.

The dissociation buffer may be a PBS buffer which has a pH in the range as indicated herein. For instance a PBS buffer containing about 1.5% BSA and 0.2 M glycine-acetate pH 2.5, or 140 mM NaCl and 0.58% acetic acid is suitable.

Incubation for several minutes, for instance such as 10 to 30, e.g., 20 minutes at a temperature in the range of 20 to 40° C. has proven sufficient.

Concentration can be achieved in a manner known per se, for instance by passing the sample over a Centriprep YM30 (Amincon Inc.).

In one embodiment of the present invention, the immunogenic products of the invention are coated on a solid phase. The sample (e.g., whole blood, cerebrospinal fluid, serum, etc.) is then contacted with the solid phase. If the antibodies, e.g. the auto-antibodies, are present in the sample, such antibodies bind to the immunogenic product on the solid phase and are then detected by either a direct or indirect method. The direct method comprises simply detecting presence of the complex itself and thus presence of the antibodies. In the indirect method, a conjugate is added to the bound antibody. The conjugate comprises a second antibody, which binds to the first bound antibodies, attached to a signal-generating compound or label. Should the second antibody bind to a bound first antibody, the signal-generating compound generates a measurable signal. Such a signal then indicates presence of the first antibodies in the sample.

Examples of solid phases used in diagnostic immunoassays are porous and non-porous materials, latex particles, magnetic particles, microparticles (see U.S. Pat. No. 5,705,330), beads, membranes, microtiter wells and plastic tubes. The choice of the solid phase material and the method of labeling the antigen or antibodies present in the conjugate, if desired, are determined based upon desired assay format performance characteristics.

As noted herein, the conjugate (or indicator reagent) will comprise an antibody (or perhaps anti-antibodies, depending upon the assay), attached to a signal-generating compound or “label”. This signal-generating compound or label is itself detectable or may be reacted with one or more additional compounds to generate a detectable product. Examples of signal-generating compounds are described herein and in particular include chromophores, radioisotopes (e.g., 125I, 131I, 32P, 3H, 35S and 14C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Kits are also included within the scope of the present invention. More specifically, the present invention includes kits for determining the presence of antibodies such as auto-antibodies in a subject. In particular, a kit for determining the presence of said antibodies in a sample comprises a) an immunogenic product as defined herein; and optionally b) a conjugate comprising an antibody attached to a signal generating compound capable of generating a detectable signal. The kit may also contain a control or calibrator which comprises a reagent which binds to the antigen.

The present invention also includes another type of kit for detecting antibodies such as auto-antibodies in a sample. The kit may comprise a) an anti-antibody specific for the antibody of interest, and b) an immunogenic product as defined herein. A control or calibrator comprising a reagent which binds to the immunogenic product may also be included. More specifically, the kit may comprise a) an anti-antibody specific for the auto-antibody and b) a conjugate comprising the immunogenic product, the conjugate being attached to a signal generating compound capable of generating a detectable signal. Again, the kit may also comprise a control or calibrator comprising a reagent which binds to the antigen.

The kit may also comprise one container such as a vial, bottle or strip, with each container with a pre-set solid phase, and other containers containing the respective conjugates. These kits may also contain vials or containers of other reagents needed for performing the assay, such as washing, processing and indicator reagents.

The immunogenic products of the invention are also useful for providing agents that are capable of binding to the immunogenic products. Such agents include, e.g., antibodies (hereinafter also referred to as anti-product antibody), non-antibody binding molecules (such as affibodies, affilin molecules, AdNectins, Anticalins, DARPins, domain antibodies, evibodies, knotins, Kunitz-type domains, maxibodies, tetranectins, trans-bodies, and V(NAR)s, as described, for instance, in the Handbook of Therapeutic Antibodies, ed. by Stefan Dubel, Volume II, Chapter 7, Wiley-VCH Verlag GmbH & Co. KGaA, Weinheim, 2007), aptamers or small-molecular weight compounds.

In one aspect, the invention thus relates to the use of the immunogenic products for screening an agent that is capable of binding to the immunogenic product. Accordingly, the present invention also relates to a method of identifying an agent capable of binding to an immunogenic product as disclosed herein, which method comprises the steps of: a) exposing one or more agents of interest to the product for a time and under conditions sufficient for the one or more agents to bind to the product; and b) identifying those agents which bind to the product.

Said agent may be selected from the group consisting of an antibody, a non-antibody binding molecule, an aptamer or a small molecular weight compound.

In another aspect, the invention relates to the use of the immunogenic products for enriching an agent that is capable of binding to the immunogenic product in a preparation comprising said agent. Accordingly, the invention also relates to a method of enriching such an agent in a preparation comprising said agent, which method comprises the steps of: a) exposing to the immunogenic product the preparation comprising the agent that is capable of binding to the immunogenic product for a time and under conditions sufficient for the agent to bind to the immunogenic product; and b) obtaining the agent in enriched form. More particularly, the immunogenic product can be immobilized (for instance on a resin), which allows the agent to be captured. Obtaining the agent in enriched form may then comprise desorbing the captured agent, preferably in such a way that desorbing the captured agent comprises contacting the captured agent with a high salt buffer or an acidic solution. This method can, for instance, be used for enriching auto-antibodies as described herein by subjecting commercial immunoglobulin preparations like IVIG or Octagam® (Octapharma Inc. Vienna, Austria) to this method. It is believed that these immunoglobulin preparations contain auto-antibodies to Aβ, and by treating subjects one raises the level of anti-AB antibodies in their body. A preparation that is enriched for said auto-antibodies would be expected to be more efficacious.

In a further aspect, the invention thus relates to the use of the immunogenic products for providing an antibody that binds to the immunogenic products. Accordingly, the invention provides a method of providing an antibody capable of binding to an immunogenic product of the invention, which comprises
i) providing an antigen comprising the product;
ii) exposing an antibody repertoire to said antigen; and
iii) selecting from said repertoire an antibody which binds to the product.

Here it is to be understood that a “potential antibody repertoire” refers to any library, collection, assembly or set of amino acid or corresponding nucleic acid sequences or to any generator of such a library, collection, assembly or set of amino acid sequences that can be used for producing an antibody repertoire in vivo or in vitro. In a preferred embodiment of the invention, the generator is the adaptive immune system of an animal, in particular the antigen-producing part of the immune system of a mammal which generates antibody diversity by a recombination process well known to those skilled in the art. In another preferred embodiment of the invention, the generator is a system for the spawning of random nucleic acid sequences which can then, by insertion into a suitable antibody framework, be used to produce an antibody repertoire in vitro.

In a preferred embodiment of the invention, the antibody repertoire or potential antibody repertoire is exposed to the antigen in vivo by immunizing an organism with said antigen. In another preferred embodiment of the invention, the potential antibody repertoire is a library of suitable nucleic acids which is exposed to the antibody by in vitro affinity screening as described in the art, e.g. a phage display and panning system.

In another aspect, the invention also provides antibodies that bind to the immunogenic products as defined herein.

In a preferred embodiment of the invention, the antibody is obtainable by a method comprising selecting the antibody from a repertoire or potential repertoire as described herein.

According to a particularly preferred embodiment, the present invention provides immunogenic product-specific antibodies. These include in particular antibodies having a comparatively smaller affinity for both the monomeric and fibrillomeric forms of Aβ peptide than for the immunogenic product of the invention. In certain embodiments, an antibody is said to specifically bind an antigen when it preferentially recognizes its target antigen in a complex mixture of proteins and/or macromolecules.

In a preferred embodiment of the invention, the affinity of the antibody to the immunogenic product is at least 2 times, e. g. at least 3 times or at least 5 times, preferably at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times greater than the binding affinity of the antibody to a monomeric Aβ(1-42).

In a preferred embodiment of the invention, the affinity of the antibody to the immunogenic product is at least 2 times, e. g. at least 3 times or at least 5 times, preferably at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times greater than the binding affinity of the antibody to a monomeric Aβ(1-40).

Expediently, the antibody of the present invention binds to one or, more preferably, both monomers with low affinity, most preferably with a K_{D} of 1×10⁻⁸ M or smaller affinity, e. g. with a K_{D} of 3×10⁻⁸ M or smaller affinity, with a K_{D} of 1×10⁻⁷ M or smaller affinity, e. g. with a K_{D} of 3×10⁻⁷ M or smaller affinity, or with a K_{D} of 1×10⁻⁶ M or smaller affinity, e. g. with a K_{D} of 3×10⁻⁵ M or smaller affinity, or with a K_{D} of 1×10⁻⁵ M or smaller affinity.

In a preferred embodiment of the invention, the affinity of the antibody to the immunogenic product is at least 2 times, e. g. at least 3 times or at least 5 times, preferably at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times greater than the binding affinity of the antibody to a fibrillomeric Aβ(1-42).

In a preferred embodiment of the invention, the affinity of the antibody to the immunogenic product is at least 2 times, e. g. at least 3 times or at least 5 times, preferably at least 10 times, e. g. at least 20 times, at least 30 times or at least 50 times, more preferably at least 100 times, e. g. at least 200 times, at least 300 times or at least 500 times, and even more preferably at least 1000 times, e. g. at least 2000 times, at least 3000 times or at least 5000 times, even more preferably at least 10000 times, e. g. at least 20000 times, at least 30000 or at least 50000 times, and most preferably at least 100000 times greater than the binding affinity of the antibody to a fibrillomeric Aβ(1-40).

Expediently, the antibody of the present invention binds to one or, more preferably, both fibrils with low affinity, most preferably with a K_{D} of 1×10⁻⁸ M or smaller affinity, e. g. with a K_{D} of 3×10⁻⁸ M or smaller affinity, with a K_{D} of 1×10⁻⁷ M or smaller affinity, e. g. with a K_{D} of 3×10⁻⁷ M or smaller affinity, or with a K_{D} of 1×10⁻⁶ M or smaller affinity, e. g. with a K_{D} of 3×10⁻⁵ M or smaller affinity, or with a K_{D} of 1×10⁻⁵ M or smaller affinity.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such functional fragment, mutant, variant, or derivative antibody formats are known in the art. Nonlimiting embodiments of which are discussed below. A “full-length antibody”, as used herein, refers to an Ig molecule comprising four polypeptide chains, two heavy chains and two light chains. The chains are usually linked to one another via disulfide bonds. Each heavy chain is comprised of a heavy chain variable region (also referred to herein as “variable heavy chain”, or abbreviated herein as HCVR or VH) and a heavy chain constant region. The heavy chain constant region is comprised of three domains, CH1, CH2 and CH3. Each light chain is comprised of a light chain variable region (also referred to herein as “variable light chain”, or abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG3, IgG4, IgA1 and IgA2) or subclass.

The terms “antigen-binding portion” of an antibody (or simply “antibody portion”), “antigen-binding moiety” of an antibody (or simply “antibody moiety”), as used herein, refers to one or more fragments of an antibody that retain the ability to specifically bind to an antigen (i.e. the immunogenic product of the invention), i.e. are functional fragments of an antibody. It has been shown that the antigen-binding function of an antibody can be performed by one or more fragments of a full-length antibody. Such antibody embodiments may also be bispecific, dual specific, or multi-specific, specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341: 544-546, 1989; Winter et al., WO 90/05144 A1, herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 242: 423-426, 1988; and Huston et al., Proc. Natl. Acad. Sci. USA 85: 5879-5883, 1988). Such single chain antibodies are also encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al., Proc. Natl. Acad. Sci. USA 90: 6444-6448, 1993; Poljak et al., Structure 2: 1121-1123, 1994). Such antibody binding portions are known in the art (Kontermann and Dubel eds., Antibody Engineering, Springer-Verlag. New York. 790 pp., 2001, ISBN 3-540-41354-5).

The term “antibody”, as used herein, also comprises antibody constructs. The term “antibody construct” as used herein refers to a polypeptide comprising one or more of the antigen-binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger et al., Proc. Natl. Acad. Sci. USA 90: 6444-6448, 1993; Poljak et al., Structure 2: 1121-1123, 1994).

An immunoglobulin constant domain refers to a heavy or light chain constant domain. Human IgG heavy chain and light chain constant domain amino acid sequences are known in the art.

Still further, a binding protein of the present invention (e.g. an antibody) may be part of a larger immunoadhesion molecule, formed by covalent or noncovalent association of the binding protein of the invention with one or more other proteins or peptides. Examples of such immunoadhesion molecules include the use of the streptavidin core region to make a tetrameric scFv molecule (Kipriyanov et al., Human Antibodies and Hybridomas 6: 93-101, 1995) and use of a cysteine residue, a marker peptide and a C-terminal polyhistidine tag to make bivalent and biotinylated scFv molecules (Kipriyanov et al., Mol. Immunol. 31: 1047-1058, 1994). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities. An isolated antibody that specifically binds the immunogenic product of the invention may, however, have cross-reactivity to other antigens, such as Aβ globulomers, e.g. Aβ(20-42) globulomer or other Aβ forms. Moreover, an isolated antibody may be substantially free of other cellular material and/or chemicals and/or any other targeted Aβ form.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g. mutations introduced by random or site-specific mutagenesis in vitro or by somatic mutation in vivo), for example in the CDRs and in particular in CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further in Section B, below), antibodies isolated from a recombinant, combinatorial human antibody library (Hoogenboom, TIB Tech. 15: 62-70, 1997; Azzazy and Highsmith, Clin. Biochem. 35: 425-445, 2002; Gavilondo J. V., and Larrick J. W. (2002) BioTechniques 29:128-145; Hoogenboom H., and Chames P. (2000) Immunology Today 21:371-378), antibodies isolated from an animal (e.g. a mouse) that is transgenic for human immunoglobulin genes (see e.g. Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine CDRs (e.g., CDR3) in which one or more of the murine variable heavy and light chain regions has been replaced with human variable heavy and light chain sequences.

The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, for example at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, particular embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In one embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): 183-200 (2002); Marchalonis et al., Adv Exp Med Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, the term “humanized antibody” is an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one aspect, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3 and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

The term “antibody”, as used herein, also comprises multivalent binding proteins. The term “multivalent binding protein” is used in this specification to denote a binding protein comprising two or more antigen binding sites. The multivalent binding protein is engineered to have the three or more antigen binding sites, and is generally not a naturally occurring antibody. The term “multispecific binding protein” refers to a binding protein capable of binding two or more related or unrelated targets. Dual variable domain (DVD) binding proteins as used herein, are binding proteins that comprise two or more antigen binding sites and are tetravalent or multivalent binding proteins. Such DVDs may be monospecific, i.e. capable of binding one antigen or multispecific, i.e. capable of binding two or more antigens. DVD binding proteins comprising two heavy chain DVD polypeptides and two light chain DVD polypeptides are referred to a DVD Ig.

Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. DVD binding proteins and methods of making DVD binding proteins are disclosed in U.S. patent application Ser. No. 11/507,050 and incorporated herein by reference.

The term “labeled binding protein”, as used herein, refers to a binding protein with a label incorporated that provides for the identification of the binding protein. Likewise, the term “labeled antibody” as used herein, refers to an antibody with a label incorporated that provides for the identification of the antibody. In one aspect, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵5, ⁹⁰Y, ⁹⁹Tc, ¹¹¹In, ¹²⁵I, ¹³¹I, ¹⁷⁷Lu ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody”, as used herein, also comprises antibody conjugates. The term “antibody conjugate” refers to a binding protein, such as an antibody, chemically linked to a second chemical moiety, such as a therapeutic agent.

Antibodies of the present invention may be made by any of a number of techniques known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In one embodiment, the present invention provides methods of generating monoclonal antibodies as well as antibodies produced by the method comprising culturing a hybridoma cell secreting an antibody of the invention wherein, e.g., the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. Briefly, mice can be immunized with an immunogenic product of the invention. In a particular embodiment, the antigen is administered with a adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with an immunogenic product of the invention, antibodies and/or antibody-producing cells may be obtained from the animal. An anti-product antibody-containing serum is obtained from the animal by bleeding or sacrificing the animal. The serum may be used as it is obtained from the animal, an immunoglobulin fraction may be obtained from the serum, or the anti-product antibodies may be purified from the serum. Serum or immunoglobulins obtained in this manner are polyclonal, thus having a heterogeneous array of properties.

Once an immune response is detected, e.g., antibodies specific for the immunogenic product of the invention are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding the immunogenic product of the invention. Ascites fluid, which generally contains high levels of antibodies, can be generated by immunizing mice with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art (See, e.g., Harlow and Lane, supra). In a particular embodiment, the myeloma cells do not secrete immunoglobulin polypeptides (a non-secretory cell line). After fusion and antibiotic selection, the hybridomas are screened using the immunogenic product of the invention, or a portion thereof, or a cell expressing the immunogenic product of the invention. In a particular embodiment, the initial screening is performed using an enzyme-linked immunoassay (ELISA) or a radioimmunoassay (RIA). An example of ELISA screening is provided in WO 00/37504, herein incorporated by reference.

Anti-product antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a particular embodiment, the hybridomas are mouse hybridomas, as described above. In another particular embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-product antibody. Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

In another aspect of the invention, recombinant antibodies are generated from single, isolated lymphocytes using a procedure referred to in the art as the selected lymphocyte antibody method (SLAM), as described in U.S. Pat. No. 5,627,052, PCT Publication WO92/02551 and Babcock, J. S. et al. (1996) Proc. Natl. Acad. Sci. USA 93:7843-7848. In this method, single cells secreting antibodies of interest, e.g., lymphocytes derived from any one of the immunized animals described in Section 1, are screened using an antigen-specific hemolytic plaque assay, wherein the immunogenic product of the invention, or a subunit thereof, is coupled to sheep red blood cells using a linker, such as biotin, and used to identify single cells that secrete antibodies with specificity for the immunogenic product of the invention. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to Aβ(20-42) globulomer. The amplified immunoglobulin sequences further can be manipulated in vitro, such as by in vitro affinity maturation methods such as those described in PCT Publication WO 97/29131 and PCT Publication WO 00/56772.

In another embodiment of the instant invention, antibodies are produced by immunizing a non-human animal comprising some, or all, of the human immunoglobulin locus with an immunogenic product antigen. In a particular embodiment, the non-human animal is a XENOMOUSE transgenic mouse, an engineered mouse strain that comprises large fragments of the human immunoglobulin loci and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The XENOMOUSE transgenic mouse produces an adult-like human repertoire of fully human antibodies, and generates antigen-specific human monoclonal antibodies. The XENOMOUSE transgenic mouse contains approximately 80% of the human antibody repertoire through introduction of megabase sized, germline configuration YAC fragments of the human heavy chain loci and x light chain loci. See Mendez et al., Nature Genetics 15:146-156 (1997), Green and Jakobovits J. Exp. Med. 188:483-495 (1998), the disclosures of which are hereby incorporated by reference.

In vitro methods also can be used to make the antibodies of the invention, wherein an antibody library is screened to identify an antibody having the desired binding specificity. Methods for such screening of recombinant antibody libraries are well known in the art and include methods described in, for example, Ladner et al. U.S. Pat. No. 5,223,409; Kang et al. PCT Publication No. WO92/18619; Dower et al. PCT Publication No. WO91/17271; Winter et al. PCT Publication No. WO92/20791; Markland et al. PCT Publication No. WO92/15679; Breitling et al. PCT Publication No. WO93/01288; McCafferty et al. PCT Publication No. WO92/01047; Garrard et al. PCT Publication No. WO92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, US patent application publication 20030186374, and PCT Publication No. WO97/29131, the contents of each of which are incorporated herein by reference.

The recombinant antibody library may be from a subject immunized with the immunogenic product of the invention, or a portion of the immunogenic product of the invention. Alternatively, the recombinant antibody library may be from a naïve subject, i.e., one who has not been immunized with the immunogenic product of the invention, such as a human antibody library from a human subject who has not been immunized with the immunogenic product of the invention. Antibodies of the invention are selected by screening the recombinant antibody library with the peptide comprising the immunogenic product of the invention to thereby select those antibodies that recognize the immunogenic product of the invention and discriminate other Aβ-forms such as Aβ(1-40) and Aβ(1-42) monomer, Aβ-fibrils and sAPPα. Methods for conducting such screening and selection are well known in the art, such as described in the references in the preceding paragraph.

In one aspect, the invention pertains to an isolated antibody, or an antigen-binding portion thereof, that binds the immunogenic product of the invention and discriminates Aβ(1-40) and Aβ(1-42) monomer, Aβ-fibrils and sAPPα. According to one aspect, the antibody is a neutralizing antibody. In various embodiments, the antibody is a recombinant antibody or a monoclonal antibody.

For example, the antibodies of the present invention can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO90/02809; WO91/10737; WO92/01047; WO92/18619; WO93/11236; WO95/15982; WO95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Alternative to screening of recombinant antibody libraries by phage display, other methodologies known in the art for screening large combinatorial libraries can be applied to the identification of dual specificity antibodies of the invention. One type of alternative expression system is one in which the recombinant antibody library is expressed as RNA-protein fusions, as described in PCT Publication No. WO 98/31700 by Szostak and Roberts, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

In another approach the antibodies of the present invention can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present invention include those disclosed Wittrup, et al. U.S. Pat. No. 6,699,658 incorporated herein by reference.

Antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is possible to express the antibodies of the invention in either prokaryotic or eukaryotic host cells. According to a particular aspect of the invention, expression of antibodies is performed using eukaryotic cells, for example mammalian host cells, because such eukaryotic cells (and in particular mammalian cells) are more likely than prokaryotic cells to assemble and secrete a properly folded and immunologically active antibody.

According to one aspect, mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibodies genes are introduced into mammalian host cells, the antibodies are produced by culturing the host cells for a period of time sufficient to allow for expression of the antibodies in the host cells or secretion of the antibodies into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In a particular system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art and discussed in detail herein. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entireties. In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454 which are incorporated herein by reference in their entireties) by splicing genes from a mouse antibody molecule of appropriate antigen specificity together with genes from a human antibody molecule of appropriate biological activity can be used.

CDR-grafted antibodies of the invention comprise heavy and light chain variable region sequences from a human antibody wherein one or more of the CDR regions of VH and/or VL are replaced with CDR sequences of the murine antibodies of the invention. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, the human variable framework chosen to replace the murine variable framework apart from the CDRs have for example at least a 65% sequence identity with the murine antibody variable region framework. The human and murine variable regions apart from the CDRs have for example at least 70%, least 75% sequence identity, or at least 80% sequence identity. Methods for producing chimeric antibodies are known in the art and discussed in detail herein. (also see EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352).

Humanized antibodies are antibody molecules from non-human species antibody that binds the desired antigen having one or more complementarity determining regions (CDRs) from the non-human species and framework regions from a human immunoglobulin molecule.

Known human Ig sequences are disclosed, e.g., www.ncbi.nlm.nih.gov/entrez-/query.fcgi; www.atcc.org/phage/hdb.html; www.sciquest.com/; www.abcam.com/; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kuby05.htm; www.Iibrary.thinkquest.org/12429/Immune/Antibody.html; www.hhmi.org/grants/lectures/1996/vlab/; www.path.cam.ac.uk/.about.mrc7/m-ikeimages.html; www.antibodyresource.com/; mcb.harvard.edu/BioLinks/Immuno-logy.html.www.immunologylink.com/; pathbox.wustl.edu/.about.hcenter/index.-html; www.biotech.ufl.edu/.about.hcl/; www.pebio.com/pa/340913/340913.html-; www.nal.usda.gov/awic/pubs/antibody/; www.m.ehime-u.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.uni-marburg.de/.about.rek/AEP-Start.html; baserv.uci.kun.nl/.about.jraats/linksl.html; www.recab.uni-hd.de/immuno.bme.nwu.edu/; www.mrc-cpe.cam.ac.uk/imt-doc/pu-blic/INTRO.html; www.ibt.unam.mx/vir/V_mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOsem-inar/Slide01.html; www.cryst.bbk.ac.uk/.about.ubcg07s/; www.nimr.mrc.ac.uk/CC/ccaewg/ccaewg.htm; www.path.cam.ac.uk/.about.mrc7/h-umanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Web-pages/Pept/spottech.html; www.jerini.de/fr roducts.htm; www.patents.ibm.com/ibm.html.Kabat et al., Sequences of Proteins of Immunological Interest, U.S. Dept. Health (1983), each entirely incorporated herein by reference. Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al., Nature 321:522 (1986); Verhoeyen et al., Science 239:1534 (1988), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska. et al; PNAS 91:969-973 (1994); PCT publication WO 91/09967, PCT: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

In certain embodiments, the antibody comprises a heavy chain constant region, such as an IgG1, IgG2, IgG3, IgG4, IgA, IgE, IgM or IgD constant region. According to one aspect, the heavy chain constant region is an IgG1 heavy chain constant region or an IgG4 heavy chain constant region. According to a further aspect, the antibody comprises a light chain constant region, either a kappa light chain constant region or a lambda light chain constant region. According to one aspect, the antibody comprises a kappa light chain constant region. An antibody portion can be, for example, a Fab fragment or a single chain Fv fragment.

Replacements of amino acid residues in the Fc portion to alter antibody effector function are known in the art (Winter, et al. U.S. Pat. Nos. 5,648,260 and 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment provides a labeled antibody wherein an antibody of the invention is derivatized or linked to another functional molecule (e.g., another peptide or protein). For example, a labeled antibody of the invention can be derived by functionally linking an antibody of the invention (by chemical coupling, genetic fusion, noncovalent association or otherwise) to one or more other molecular entities, such as another antibody (e.g., a bispecific antibody or a diabody), a detectable agent, a pharmaceutical agent, and/or a protein or peptide that can mediate association of the antibody with another molecule (such as a streptavidin core region or a polyhistidine tag).

Useful detectable agents with which an antibody of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

Another embodiment of the invention provides a crystallized antibody. According to one aspect, the invention relates to crystals of whole anti-Aβ(20-42) globulomer antibodies and fragments thereof as disclosed herein, and formulations and compositions comprising such crystals. According to a further aspect, the crystallized antibody has a greater half-life in vivo than the soluble counterpart of the antibody. According to a further aspect, the antibody retains biological activity after crystallization.

Crystallized antibody of the invention may be produced according methods known in the art and as disclosed in WO02/072636, incorporated herein by reference.

Another embodiment of the invention provides a glycosylated antibody wherein the antibody comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins.

Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick, S. C., et al., Exp. Med. (1988) 168:1099-1109; Wright, A., et al., EMBO J. (1991) 10:2717 2723).

One aspect of the present invention is directed to generating glycosylation site mutants in which the O- or N-linked glycosylation site of the antibody has been mutated. One skilled in the art can generate such mutants using standard well-known technologies. The creation of glycosylation site mutants that retain the biological activity but have increased or decreased binding activity is another object of the present invention.

In still another embodiment, the glycosylation of the antibody of the invention is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region glycosylation sites to thereby eliminate glycosylation at that site. Such aglycosylation may increase the affinity of the antibody for antigen. Such an approach is described in further detail in International Appln. Publication No. WO03/016466A2, and U.S. Pat. Nos. 5,714,350 and 6,350,861, each of which is incorporated herein by reference in its entirety.

Additionally or alternatively, a modified antibody of the invention can be made that has an altered type of glycosylation, such as a hypofucosylated antibody having reduced amounts of fucosyl residues or an antibody having increased bisecting GIcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent NO.: EP1,176,195; International Appln. Publication Nos. WO03/035835 and WO99/54342 80, each of which is incorporated herein by reference in its entirety.

Protein glycosylation depends on the amino acid sequence of the protein of interest, as well as the host cell in which the protein is expressed. Different organisms may produce different glycosylation enzymes (e.g., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. According to one aspect, the glycosylated antibody comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S Patent Application Publication Nos. 20040018590 and 20020137134; and WO05/100584).

Another embodiment is directed to an anti-idiotypic (anti-Id) antibody specific for such antibodies of the invention. An anti-Id antibody is an antibody, which recognizes unique determinants generally associated with the antigen-binding region of another antibody. The anti-Id can be prepared by immunizing an animal with the antibody or a CDR containing region thereof. The immunized animal will recognize, and respond to the idiotypic determinants of the immunizing antibody and produce an anti-Id antibody. The anti-Id antibody may also be used as an “immunogen” to induce an immune response in yet another animal, producing a so-called anti-anti-Id antibody.

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. According to a further aspect, the protein having a particularly selected novel glycosylation pattern exhibits improved or altered biological properties.

In a further aspect, the invention also relates to the use of the immunogenic product of the invention for providing an aptamer that binds to the immunogenic product (hereinafter also referred to as anti-product aptamer). Accordingly, the invention relates also to a method for providing an aptamer having specificity for the immunogenic product as defined herein, which method comprises at least the steps of
a) providing a binding target comprising the immunogenic product;
b) exposing an aptamer repertoire or potential aptamer repertoire to said binding target; and
c) selecting from said repertoire an aptamer which specifically binds to said immunogenic product.

An “aptamer” herein refers to oligonucleic acid or peptide molecules that are capable of specific, non-covalent binding to its target. Apatmer preferably comprise peptide, DNA or RNA sequence, more preferably peptide, DNA or RNA sequence of about 3 to 100 monomers, which may at one end or both ends be attached to a larger molecule, preferably a larger molecule mediating biochemical functions, more preferably a larger molecule inducing inactivation and/or degradation, most preferably ubiquitin, or preferably a larger molecule facilitating destruction, more preferably an enzyme or a fluorescent protein.

Here it is to be understood that a “potential aptamer repertoire” refers to any library, collection, assembly or set of amino acid sequences or nucleic acid sequences or to any generator of such a library, collection, assembly or set of amino acid sequences that can be used for producing an aptamer repertoire in vivo or in vitro.

In another aspect, the invention also provides aptamers that bind to the immunogenic products as defined herein.

In a preferred embodiment of the invention, the aptamer is obtainable by a method comprising selecting the aptamer from a repertoire or potential repertoire as described herein.

According to a particularly preferred embodiment, the present invention provides immunogenic product-specific aptamers. These include in particular aptamers having a comparatively smaller affinity for both the monomeric and fibrillomeric forms of Aβ peptide than for the immunogenic product of the invention.

The agents that are capable of binding to the immunogenic product of the invention also have many potential applications, some of which are described in the following. They are especially useful for therapeutic and diagnostic purposes.

The present invention further relates to a molecule comprising an amino acid sequence identical to a portion (X—Y) of an amino acid sequence selected from the group consisting of:

[SEQ ID NO: 13; Aβ(1-43)F19A]
D₁A₂E₃F₄R₅H₈D₇S₈G₉Y₁₀E₁₁V₁₂H₁₃H₁₄Q₁₅K₁₈L₁₇V₁₈A₁₉F₂₀A₂₁E₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 14; Aβ(1-43)F20A]
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 15; Aβ(1-43)E22A]
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 16; Aβ(1-43)E22F]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁F₂₂D₂₃V₂₄G₂₅S₂₆N₂₇K₂₈
G₂₉A₃₀I₃₁I₃₂G₃₄L₃₄M₃₅V₃₆G₃₇G₃₈V₃₉V₄₀I₄₁A₄₂T₄₃;
[SEQ ID NO: 17; Aβ(1-43)E22V]
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁V₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 18; Aβ(1-43)E22L]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 19; Aβ(1-43)D23K]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂K₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 20; Aβ(1-43)D23L]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂L₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 21; Aβ(1-43)G25V]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄V₂₅S₂₆N₂₇K₂₈
G₂₉A₃₀I₃₁I₃₂G₃₃L₃₄M₃₅V₃₆G₃₇G₃₈V₃₉V₄₀₁₄₁A₄₂T₄₃;
[SEQ ID NO: 22; Aβ(1-43)A30G]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄G₂₅S₂₆N₂₇K₂₈
G₂₉G₃₀I₃₁I₃₂G₃₃L₃₄M₃₅V₃₆G₃₇G₃₈V₃₉V₄₀I₄₁A₄₂T₄₃;
[SEQ ID NO: 23; Aβ(1-43)F20G E22A]
D₁A₂E₃F₄R₅H₆D₇S₈G₉Y₁₀E₁₁V₁₂H₁₃H₁₄Q₁₅K₁₆L₁₇V₁₈F₁₉G₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 24; Aβ(1-43)F20A I31A]
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀A₃₁I₃₂G₃₃L₃₄M₃₅V₃₆G₃₇G₃₈V₃₉V₄₀I₄₁A₄₂T₄₃;
[SEQ ID NO: 25; Aβ(1-43)F20C I31C]
D₁A₂E₃F₄R₅H₆D₇S₈G₉Y₁₀E₁₁V₁₂H₁₃H₁₄Q₁₅K₁₆L₁₇V₁₈F₁₉C₂₀A₂₁E₂₂D₂₃V₂₄G₂₅S₂₆N₂₇K₂₈
G₂₉A₃₀C₃₁I₃₂G₃₃L₃₄M₃₅V₃₆G₃₇G₃₈V₃₉V₄₀I₄₁A₄₂T₄₃;
[SEQ ID NO: 26; Aβ(1-43)A21Q E22L]
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 27; Aβ(1-43)A21L E22Q]
D₁A₂E₃F₄R₅H₆D₇S₈G₉Y₁₀E₁₁V₁₂H₁₃H₁₄Q₁₅K₁₆L₁₇V₁₈F₁₉F₂₀L₂₁Q₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 28; Aβ(1-43)A21Q D23N]
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁E₂₂N₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
[SEQ ID NO: 29; Aβ(1-43)E22A G25A]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄A₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃;
and
[SEQ ID NO: 30; Aβ(1-43)E22A S26A]
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄G₂₅A₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,

with X being selected from the group consisting of the numbers 1 . . . 18, 4 . . . 18, 12 . . . 18 or being 18 and Y being selected from the group consisting of the numbers 33 . . . 43, 33 . . . 42, 33 . . . 41, or 33 . . . 40; or a crosslinked derivative thereof, wherein at least 2 non-contiguous residues of the amino acid sequence are covalently linked with each other.

In a particular embodiments of said molecule or crosslinked derivative thereof, (X—Y) is selected from the group consisting of (1-42), (4-42), (12-42), or (18-42).

### EXAMPLES

Aβ mutein peptides were synthesized using standard methods.

### Example 1: Preparation of as Mutein Oligomer

a) Aβ(1-42) E22A Mutein Oligomer:

The Aβ(1-42) E22A peptide which was obtained via peptide synthesis (MoBiTec GmbH, Gottingen, Germany) was suspended in 100% 1,1,1,3,3,3-hexafluoro-2-propanol (HFIP) at 6 mg/ml and incubated for complete solubilization under shaking at 37° C. for 2.5 h. The HFIP acts as a hydrogen-bond breaker and is used to eliminate pre-existing structural inhomogeneities in the Aβ peptide. HFIP was removed by evaporation in a SpeedVac and Aβ(1-42) E22A resuspended at a concentration of 5 mM in dimethylsulfoxide and sonicated for 20 s. The HFIP-pre-treated Aβ(1-42) E22A was diluted in phosphate-buffered saline (PBS) (20 mM NaH₂PO4, 140 mM NaCl, pH 7.4) to 400 μM and 1/10 volume 2% sodium dodecyl sulfate (SDS) (in H₂O) added (final concentration of 0.2% SDS). The solution was incubated for 6 h at 37° C. Next, the solution was further diluted with three volumes of H₂O and incubated for 18 h at 37° C. which lead to the generation of the Aβ(1-42) E22A mutein oligomer. After centrifugation at 3000 g for 20 min the sample was two times dialyzed at room temperature against 0.5 l of buffer (5 mM sodium phosphate, 35 mM NaCl, pH 7.4) in a dialysis tube for 2.5 h. The dialysate was 15-fold concentrated by ultrafiltration (30 kDa cut-off), centrifuged at 10,000 g for 5 min and the supernatant comprising the Aβ(1-42) E22A mutein oligomer withdrawn.

b) Truncated as E22A Mutein Oligomer:

30 μl of a 1 mg/ml thermolysin solution (Sigma) in H₂O were added to 0.8 ml of Aβ(1-42) E22A mutein oligomer preparation prepared according to Example 1a. The reaction mixture was shaken at 30° C. for 20 h. Then, 4 μl of a 100 mM EDTA solution, pH 7.4, in water were added and the mixture was furthermore adjusted to an SDS content of 0.1% with 8 μl of a 10% strength SDS solution. The reaction mixture was shaken for 10 min at room temperature and then dialyzed at room temperature against 0.5 l of buffer (5 mM sodium phosphate, 35 mM NaCl, 0.1% SDS, pH 7.4) in a dialysis tube for 6 h and after exchange of the dialysis buffer for further 20 h. The dialysate was removed and stored at −80° C. for further usage.

c) Ethanol Precipitated Truncated Aβ E22A Mutein Oligomer:

For the use of antigen in active immunization the truncated Aβ E22A mutein oligomer from example 1b was ethanol precipitated. To this end one part (v/v; e.g. 1 ml) of truncated Aβ mutein oligomer with a concentration of 0.5-10 mg/ml was thawed at room temperature. Then 8 parts (v/v; e.g. 8 ml) ice cold ethanol were added to the sample. The sample was briefly mixed and 1 part (v/v, based on the initial volume of truncated Aβ mutein oligomer; e.g. 1 ml) 10×-PBS (Fa. Gibco, cat. no. 14200-067) were added. Subsequently the sample was briefly mixed again and incubated for 30 min in an ice bath. The sample was centrifuged for 20 min at 3000 g and the supernatant was discarded. The remaining pellet was suspended with an appropriate volume 5 mM NaH₂PO₄, 35 mM NaCl, pH 7.4 to a final concentration of 1 mg/ml. After 10 min cooling in an ice bath the sample was sonicated with an ultra sonicator (UP 200s Dr. Hielscher GmbH) for 5×3 seconds at 0° C. in an ice bath (with intermediate cooling for 10 sec on ice) at 50% of the maximum power. After this step the sample was aliquoted and frozen at −80° C. until further use.

Using the procedures of examples 1a, 1b and 1c, the Aβ mutein oligomers listed in table 2 below were prepared, subjected to proteolytic digestion and precipitated from ethanol.

### Example 2: Surface-Enhanced Laser Desorption Ionization-Mass Spectrometry (SELDI-MS) Semi-Quantitative Determination of Aβ Peptide Composition of Truncated as Mutein Oligomers

1 μl truncated Aβ E22A mutein oligomer from example 1b was diluted with 249 μl 50% acetonitrile; 0.5% TFA (500 μl acetonitrile+500 μl 1% TFA). 1 μl sample was spotted onto a H4 Protein Chip Array (BioRad; Cat. no. C57-30028). The spots were allowed to dry on a warm incubator plate at 40° C. CHCA-solution was prepared as follows: 5 mg CHCA (BioRad; Cat. no. C30-00001) were dissolved in 150 μl acetonitrile+150 μl 1% TFA=stock solution (stored at −20° C.). 10 μl of the stock solution were diluted with 20 μl acetonitrile and 20 μl 1% TFA to give the working CHCA-solution. 2 μl of the working CHCA-solution was applied onto the spots. The spots were allowed to dry on a warm incubator plate at 40° C. and analyzed by SELDI-MS (Surface-Enhanced Laser Desorption Ionization-Mass Spectrometry; BioRad, Protein chip SELDI system enterprise edition) using the following parameters: mass range: 500 to 10000 Da; focus mass: 2220 Da; matrix attenuation: 500 Da; sampling rate: 400 MHz; warming shots: 2 with energy: 1100 nj; data shots: 10 with energy 1000 nJoule; partition 1 of 3. The truncated forms of the other Aβ mutein oligomers listed in table 2 were subjected to the same procedure.

It was observed that the Aβ peptide compositions of the truncated Aβ mutein oligomers varies. In table 2, the amounts of characteristic Aβ-peptide fragments for each truncated Aβ mutein oligomer are indicated.

TABLE 2
Seldi-MS analysis of different truncated Aβ mutein oligomers

| Mutein | Seldi-MS |
|---|---|
| Aβ(1-42) V18A | <5% Aβ4-42 |
| Aβ(1-42) F19A | <5% Aβ4-42 |
| Aβ(1-42) F20A | ca. 5% Aβ4-42 |
| Aβ(1-42) A21G | <5% Aβ4-42 |
| Aβ(1-42) E22A | >50% Aβ24-42 |
| Aβ(1-42) D23A | >80% Aβ23-42 |
| Aβ(1-42) V24A | <5% Aβ24-42 |
| Aβ(1-42) G25A | ca. 10% Aβ4-42 |
| Aβ(1-42) S26A | 60% Aβ20-42; 40% Aβ24-42 |
| Aβ(1-42) N27A | <5% Aβ24-42 |
| Aβ(1-42) K28A | ca. 10% Aβ4-42 |
| Aβ(1-42) G29A | ca. 5% Aβ20-42; ca. 5% Aβ4-42 |
| Aβ(1-42) A30G | ca. 20% Aβ24-42; ca. 10% Aβ4-42 |
| Aβ(1-42) I31A | ca. 40% Aβ4-42 |
| Aβ(1-42) I32A | ca. 10% Aβ4-42 |
| Aβ(1-42) G33A | ca. 15% Aβ24-42 |
| Aβ(1-42) A21Q | ca. 15% Aβ24-42 |
| Aβ(1-42) A21L | ca. 50% Aβ21-42, <5% Aβ4-30 |
| Aβ(1-42) E22G | <5% Aβ24-42 |
| Aβ(1-42) E22Q | ca. 20% Aβ4-42 (old procedure) |
| Aβ(1-42) E22K | ca. 20% Aβ4-42 |
| Aβ(1-42) E22D | <5% Aβ4-42 |
| Aβ(1-42) E22L | >80% Aβ22-42; 20% Aβ4-30 + Aβ4-33 |
| Aβ(1-42) D23N | >50% Aβ24-42 |
| Aβ(1-42) D23L | >80% Aβ23-42; 20% Aβ4-30 + Aβ4-33 |
| Aβ(1-42) E22F | 10% Aβ20-42; 60% Aβ22-42; 30% Aβ24-42 |
| Aβ(1-42) E22V | 20% Aβ22-42; 20% Aβ24-42; 60% |
| | different unknown peaks |
| Aβ(1-42) D23K | 30% Aβ24-42 |
| Aβ(1-42) D23V | no peaks detectible |
| Aβ(1-42) G25V | 60% Aβ20-42; 40% Aβ24-42 |
| Aβ(1-42) G25T | 70% Aβ20-42; 30% Aβ24-42 |
| Aβ(1-42) S26L | 20% Aβ24-42 |
| Aβ(1-42) A21G, E22Q | <5% Aβ4-42 |
| Aβ(1-42) A21G, E22K | <5% Aβ4-42 |
| Aβ(1-42) A21Q, E22L | >80% Aβ22-42; 20% Aβ4-30 + Aβ4-29 |
| Aβ(1-42) A21L, E22Q | >50% Aβ21-42, 30% Aβ24-42 |
| Aβ(1-42) A21G, D23N | <5% Aβ4-42 |
| Aβ(1-42) F20A, I31A | 20% Aβ4-42; 30% Aβ19-42 and |
| | Aβ20-42; 20% Aβ17-42 |
| Aβ(1-42) F20G, E22A | 10% Aβ4-42; 20% Aβ20-42 and Aβ24-42; |
| | 50% Aβ21-42 |
| Aβ(1-42) E22A, D23A | — |
| Aβ(1-42) E22A, G25A | 40% Aβ20-42; 60% Aβ24-42 |
| Aβ(1-42) E22A, S26A | 40% Aβ20-42; 60% Aβ24-42 |
| Aβ(1-42) G25A, S26A | 40% Aβ20-42; 60% Aβ24-42 |
| Aβ(0-42) F20C, I31C | >80% Aβ12-42 |

### Example 3: Size Exclusion Chromatography of Truncated as Mutein Oligomers

Size exclusion chromatography was performed using a SEC column Superose 12 HR 10/300 GL (GE Health Care, catalogue no. 17-5173-01) and a flow-rate of 0.5 ml/min. The mobile phase was 20 mM NaH₂PO₄, 140 mM NaCl, 0.5% SDS, pH 7.4. 30 μg Truncated Aβ mutein oligomer from example 1b was diluted with mobile phase to obtain 150 μl with a concentration of 200 μg/ml. 100 μl of this mixture were loaded onto the column. Peptide with extinction at 215 nm was detected.

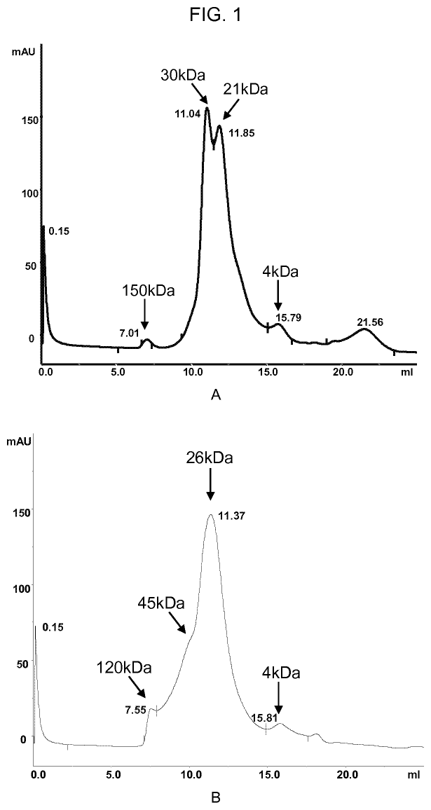

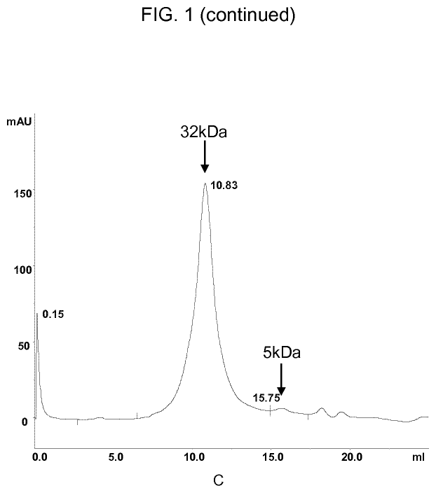

The resulting size exclusion chromatogram (FIG. 1B) for the truncated Aβ E22A mutein oligomer shows a major peak at 11.37 ml corresponding to 26 kDa and minor peaks at 45 kDa, 120 kDa and 4 kDa. The truncated Aβ F20G, E22A mutein oligomer (FIG. 1C) shows a more uniform size distribution with a major peak at 10.83 ml corresponding to 32 kDa and only a small peak at 5 kDa. As a reference, the size exclusion chromatogram of wild type Aβ(20-42) globulomer (FIG. 1A) is shown which has a major double peak at 11.04 ml and 11.85 ml corresponding to 30 kDa and 21 kDa, respectively, and minor peaks at 150 kDa and 4 kDa. Taken together, size exclusion chromatography confirmed that the oligomeric nature of the truncated Aβ mutein oligomers resembles wild type Aβ(20-42) globulomer.

### Example 4: Direct ELISA of Truncated as Mutein Oligomers

The immunoreactivity of truncated Aβ mutein oligomers from example 1b was further characterized by using the murine, Aβ(20-42) globulomer-reactive, monoclonal antibodies m7C6 and m4D10 in order to predict the propensity of truncated Aβ mutein oligomers to elicit an undesired polyclonal cross-reactivity to PF-4. The antibody m7C6 has been shown to cross-react with PF-4 while the m4D10 has been proven to not cross-react with PF-4.

Direct-ELISA protocol used for the determination of truncated Aβ mutein oligomer recognition:

Reagents:
1. F96 Cert. Maxisorp NUNC-Immuno Plate Cat. No.: 439454
2. Antigen: truncated Aβ mutein oligomer from example 1b
3. Coating buffer: 100 mM sodiumhydrogencarbonate; pH 8.2
4. Blocking reagent for ELISA; Roche Diagnostics GmbH Cat. No.: 1112589
5. PBST-Buffer: 20 mM NaH₂PO₄; 140 mM NaCl; 0.05% Tween 20; pH 7.4
6. PBST+0.5% BSA-Buffer: 20 mM NaH₂PO₄; 140 mM NaCl; 0.05% Tween 20; pH 7.4+0.5% BSA; Serva cat. 11926
7. Primary antibodies:
   Anti-Aβ mAb clone 7C6; conc.: 2.83 mg/ml OD 280 nm; stored at −80° C.
   Anti-Aβ mAb clone 4D10; conc.: 8.60 mg/ml OD 280 nm; stored at −80° C.
8. Label reagent: anti-mouse-POD conjugate; Jackson ImmunoResearch Ltd. Cat. No.: 715-035-150
9. Staining: TMB; Roche Diagnostics GmbH Cat. No.: 92817060; 42 mM in DMSO; 3% H₂O₂ in water; 100 mM sodium acetate, pH 4.9
10. Stop Solution: 2M sulfonic acid

Method Used in Preparation of Reagents:

1. Antigen Solution:

12 μg truncated Aβ mutein oligomer were diluted with 12 ml coating buffer to 1 μg/ml

2. Blocking Reagent:

Blocking reagent was dissolved in 100 ml water to prepare the blocking stock solution and aliquots of 10 ml were stored at −20° C. 3 ml blocking stock solution was diluted with 27 ml water for each plate to block.

3. Primary Antibody Dilution:
A) Anti-Aβ mAb clone 7C6 was diluted in PBST+0.5% BSA to a concentration of 100 ng/ml (=Stock solution A).
B) Anti-Aβ mAb clone 4D10 was diluted in PBST+0.5% BSA to a concentration of 100 ng/ml (=Stock solution B).

Primary Antibody Curve (Prepared for A) Clone 7C6 and B) Clone 4D10):

| No | Stock solution A) or B) | PBST + 0.5% BSA | | Final conc. |
|---|---|---|---|---|
| 1 | 2 ml 100 ng/ml | 0 | ml | 100 ng/ml |
| 2 | 0.633 ml (1) | 1.367 | ml | 31.6 ng/ml |
| 3 | 0.633 ml (2) | 1.367 | ml | 10 ng/ml |
| 4 | 0.633 ml (3) | 1.367 | ml | 3.16 ng/ml |
| 5 | 0.633 ml (4) | 1.367 | ml | 1 ng/ml |
| 6 | 0.633 ml (5) | 1.367 | ml | 0.316 ng/ml |
| 7 | 0.633 ml (6) | 1.367 | ml | 0.1 ng/ml |
| 8 | 0 ml | 2 | ml | 0.0 ng/ml |

4. Label Reagent:

Anti-mouse-POD conjugate lyophilizate was reconstituted in 0.5 ml water. 500 μl Glycerol was added and aliquots of 100 μl were stored at −20° C. for further use. The concentrated label reagent was diluted 1/10000 in PBST-buffer. The reagent was used immediately.

5. Tmb Solution:

20 ml 100 mM of sodium acetate, pH 4.9, were mixed with 200 μl TMB solution and 29.5 μl 3% H₂O₂ solution. The solution was used immediately.

Standard Plate Setup. Numbers indicate final antibody concentration in ng/ml. The standard for each antibody was run in duplicate.

| | 1 | 2 | 3 | 4 | | | | | | | | |
|---|---|---|---|---|---|---|---|---|---|---|---|---|
| | m7C6 | | m4D10 | | 5 | 6 | 7 | 8 | 9 | 10 | 11 | 12 |
| A | 100 | 100 | 100 | 100 | | | | | | | | |
| B | 31.6 | 31.6 | 31.6 | 31.6 | | | | | | | | |
| C | 10 | 10 | 10 | 10 | | | | | | | | |
| D | 3.16 | 3.16 | 3.16 | 3.16 | | | | | | | | |
| E | 1 | 1 | 1 | 1 | | | | | | | | |
| F | 0.316 | 0.316 | 0.316 | 0.316 | | | | | | | | |
| G | 0.1 | 0.1 | 0.1 | 0.1 | | | | | | | | |
| H | 0.0 | 0.0 | 0.0 | 0.0 | | | | | | | | |

Procedure:
1. 100 μl antigen solution per well were applied and incubated overnight at 4° C.
2. The antigen solution was discarded and the wells were washed three times with 250 μl PBST buffer.
3. 265 μl block solution per well were added and incubated for 2 h at room temperature.
4. The block solution was discarded and the wells were washed three times with 250 μl PBST buffer.
5. After preparation of the antibody curves, 100 μl per well of the dilution series were applied to the plate. The plate was incubated for 2 h at room temperature.
6. The antibody solution was discarded and the wells were washed three times with 250 μl PBST buffer.
7. 200 μl label solution per well were added and incubated for 1 h at room temperature.
8. The label solution was discarded and the wells were washed three times with 250 μl PBST buffer.
9. 100 μl of TMB solution were added to each well and incubated 5-15 min at room temperature.
10. Color staining was observed and 50 μl of the stop solution per well were applied.
11. The absorbance at 450 nm was measured.

Results:

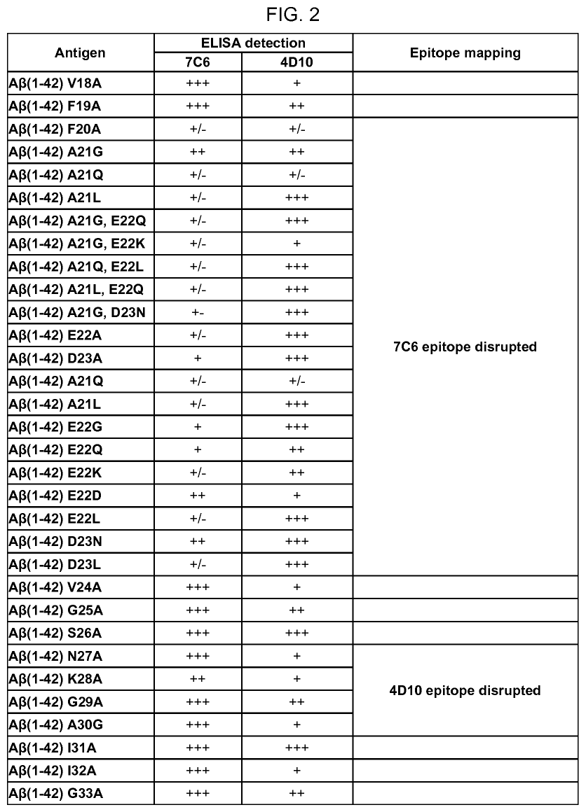

By introducing a single or double amino acid point mutation in the region of amino acid positions 20-23 the recognition of the resulting truncated Aβ mutein oligomers by antibody m7C6 was reduced, whereas the recognition by m4D10 is not reduced or was reduced only to a limited extent (see FIG. 2). In contrast, recognition of Aβ(20-42) oligomers by the non-PF-4 cross-reacting antibody 4D10 is reduced by point mutations mainly in the region of amino acid positions 27-30, although the epitope cannot be mapped so precisely. The region of reduced m7C6 recognition, but maintained m4D10 recognition, can be interpreted as comprising hot-spots that are relevant for eliciting a polyclonal immune response upon immunization with a respective Aβ(20-42) mutein oligomer that does not concomitantly elicit a PF-4 cross reactivity.

### Example 5: Truncated Aβ E22A Mutein Oligomer Induces an as Globulomer-Specific Immune Response

The antigenicity of the reactivity Aβ mutein oligomers was tested by active immunization of rodents (rabbits, mice). The polyclonal antisera obtained from said animals were affinity-purified and subsequently tested for their specificity towards different forms of Aβ using the dot blot method. The individual forms of Aβ were blotted in serial dilutions and incubated with the corresponding affinity-purified mouse antisera containing anti-Aβ antibodies produced in the immune reaction. The individual dot blots correspond to different individuals of the immunized rodents.

### Example 5A: Active Immunization of Mice with Truncated Aβ E22A Mutein Oligomer

The mice (Balb/c mice) received 30 μg of the ethanol precipitated truncated Aβ E22A mutein oligomer prepared according to example 1c and mixed with complete Freund's adjuvant, Alum adjuvant or no adjuvant subcutaneously at day 0. The mice were boosted according to the following scheme: boost 1: on day 17, boost 2: on day 35 and boost 3 on day 52. For titer determination, plasma was withdrawn 7-10 days after boost 2 and/or 3.

Preparation of Adjuvants:

Alum Adjuvant Preparation:

1 ml 1.4 M NaCl solution pH7.4 were added to 9 ml aluminum hydroxide gel (Sigma; cat. no. A8222-250 ml). The mixture was incubated for 24 h at room temperature prior to use.

Complete Freund's Adjuvant (CFA):

CFA was obtained as a ready-to-use adjuvant solution and was used for the initial immunization. For all subsequent boost immunizations Incomplete Freund's Adjuvant (IFA) was used.

No Adjuvant:

In cases where no adjuvant was used, % PBS buffer (5 mM NaH₂PO₄; 35 mM NaCl; pH7.4) was used instead.

Prior to active immunization, 100 μl of the truncated Aβ mutein oligomer (the antigen) was mixed with an equal volume of the respective adjuvants. The antigen/adjuvant mixture was incubated at room temperature for 1 h and briefly shaken. Then, the total volume of 200 μl was injected subcutaneously in the neck of the mouse. Where CFA or IFA was used as adjuvant, the antigen and CFA or IFA adjuvant solutions were mixed until a suspension had formed which was then used immediately for injection.

### Example 5B-1: Affinity Purification of Polyclonal Antibodies from Mouse Plasma Samples Via Sepharose Beads

Immobilization of Aβ(20-42) Mutein Oligomer on Sepharose Beads

Reagents:

30% Isopropanol in 1 mM HCl in H₂O (pre-cooled at 0° C. in an ice bath)

1 mM HCl in H₂O (pre-cooled at 0° C. in an ice bath)

50 mM NaHCO₃; pH 7.5 (pre-cooled at 0° C. in an ice bath)

50 mM NaHCO₃/250 mM ethanolamine; pH 7.5

¼ PBS (5 mM NaH₂PO₄; 35 mM NaCl; pH 7.4)

NHS-activated sepharose beads (Fa. GE #17-0906-01) in 100% isopropanol

TBS: (25 mM Tris; 150 mM NaCl; pH7.5)

Procedure:

2 ml NHS-activated sepharose beads (=2.8 ml suspension in isopropanol) were washed 4× with 10 ml 30% isopropanol in 1 mM HCl in H₂O (pre-cooled at 0° C. in an ice bath), 4× with 10 ml 1 mM HCl in H₂O (pre-cooled at 0° C. in an ice bath), and 4× with 10 ml 50 mM NaHCO₃, pH 7.5 (pre-cooled at 0° C. in an ice bath). 0.2 mg truncated Aβ mutein oligomer from example 1b were diluted with 50 mM NaHCO₃ pH 7.5+0.1% SDS to obtain a concentration of 0.5 mg/ml. The truncated Aβ mutein oligomer solution was added to 0.2 g of the washed NHS-activated sepharose beads, and the mixture was shaken at room temperature for 2 h. After centrifugation at 3000 g for 5 min, 1 ml 50 mM NaHCO₃/250 mM ethanolamine, pH 7.5+0.1% SDS was added to the sepharose beads, and the mixture was shaken at room temperature for 1 h. The sample was transferred into a PolyPrep Chromatography Column (Fa. Biorad #731-1550) and washed 5× with 1 ml PBS (5 mM NaH₂PO₄; 35 mM NaCl; pH7.4)+0.1% SDS, and then 5× with 1 ml TBS. After the last washing step, the sepharose beads carrying the immobilized Aβ(20-42) mutein oligomer were transferred into a 1.5 ml tube and stored at 6° C. for further use.

Immobilization of Aβ(1-42) Monomer on Sepharose Beads:

Reagents:

see above

Procedure:

2 ml NHS-activated sepharose beads (=2.8 ml suspension in isopropanol) were washed with 4× with 10 ml 30% Isopropanol in 1 mM HCl in H₂O (pre-cooled at 0° C. in an ice bath), 4× with 10 ml 1 mM HCl in H₂O (pre-cooled at 0° C. in an ice bath), and 4× with 10 ml 50 mM NaHCO₃, pH 7.5 (pre-cooled at 0° C. in an ice bath). 0.81 mg Aβ(1-42) synthetic peptide (H-1368, Bachem, Bubendorf, Switzerland) were dissolved in 80 μl 0.1% NaOH in H₂O. 50 μl of this 10 mg/ml Aβ(1-42) monomer solution were diluted with 950 μl 50 mM NaHCO₃; pH 7.5 to obtain a concentration of 0.5 mg/ml. The Aβ(1-42) monomer solution was added to 0.5 g of the washed NHS-activated sepharose beads, and the mixture was shaken at room temperature for 2 h. After centrifugation at 3000 g for 5 min, 1 ml 50 mM NaHCO₃/250 mM ethanolamine, pH 7.5 was added to the sepharose beads, and the mixture was shaken at room temperature for 1 h. The sample was filled into a PolyPrep Chromatography Column and washed 5× with 1 ml PBS (5 mM NaH₂PO₄; 35 mM NaCl; pH7.4), and then 5× with 1 ml TBS. After the last washing step, the sepharose beads carrying the Aβ(1-42) monomer were transferred into a 1.5 ml tube and stored at 6° C. for further use.

Affinity Purification of Polyclonal Antibodies from Mouse Plasma Samples

Reagents:

TBS (25 mM Tris; 150 mM NaCl; pH7.5)

Complete; Protease Inhibitor Cocktail tablets; Roche, cat. no. 11697498001

1/10 TBS (2.5 mM Tris; 15 mM NaCl; pH7.5)

elution buffer: 0.58% CH₃COOH/140 mM NaCl

neutralization buffer: 2 M Tris/HCl; pH 8.5

sepharose beads carrying immobilized truncated Aβ mutein oligomer

sepharose beads carrying Aβ(1-42) monomer

Antigen-specific antibodies generated by immunization of the mice with truncated Aβ mutein oligomer were affinity-purified using a mixture of the respective truncated Aβ mutein oligomer and monomeric Aβ(1-42) peptide as affinity capture proteins. The monomeric Aβ(1-42) peptide was used to ensure that all anti-Aβ antibodies are affinity-purified, including antibodies binding to non-globulomer epitopes, e.g. to sAPPα, monomeric or fibrillary Aβ peptide, that might potentially be present in the antisera.

Procedure:

250 μl of each mouse plasma sample were mixed with 250 μl TBS+1/50 Complete (1 tablet dissolved in 1 ml H₂O), and solution was centrifuged for 10 min at 10000 g. The supernatant was removed and 50 μl sepharose beads were added that carried the truncated Aβ mutein oligomer corresponding to the antigen used for immunization of the mouse. After 5 min shaking at room temperature, 12.5 μl sepharose beads carrying Aβ(1-42) monomer were added. The mixture was shaken for 20 h at room temperature and 1100 rpm in a Eppendorf Thermomixer Comfort. Then, the sepharose beads were transferred using 2×100 μl TBS into a PolyPrep Chromatography Column, washed 4× with 250 μl TBS and 2× with 250 μl 1/10 TBS. After the last washing step, the beads were eluted twice with 100 μl and then once with 120 μl 0.58% CH₃COOH/140 mM NaCl. The eluate (approximately 250-270 μl) was collected in a 1.7 ml tube pre-loaded with 22 μl 2M Tris/HCl, pH 8.5. After the elution step, the sample was immediately mixed and then stored at −80° C. for further use. The protein concentration of the affinity-purified polyclonal antibodies from mouse plasma was determined by measuring the absorption of each affinity purification eluate at 280 nm against a TBS only reference blank. The binding of the affinity-purified polyclonal antibodies to Aβ globulomer was confirmed via direct ELISA.

### Example 5B-2: Affinity Purification of Polyclonal Antibodies from Mouse Plasma Samples Via Magnetic Dynabeads

Immobilization of Truncated Aβ Mutein Oligomer on Tosyl-Activated Dynabeads

Reagents:

Dynabeads M-280 Tosyl activated, Invitrogen, cat. no. 142-04; 2×1E09 beads/ml

100 mM sodium borate, pH9.5

100 mM sodium borate, pH9.5+0.5% BSA

PBS (20 mM NaH₂PO₄, 140 mM NaCl, pH7.4)

PBS (20 mM NaH₂PO₄, 140 mM NaCl, pH7.4)+0.1% BSA

PBS (20 mM NaH₂PO₄, 140 mM NaCl, pH7.4)+0.1% BSA+0.02% sodium azide

Procedure:

The stock-suspension of dynabeads was homogenized by shaken carefully to prevent foaming. 66 μl suspension were removed and transferred to a 1.5 ml reaction vial. The dynabeads were washed 2×2 min with 200 μl 100 mM sodium borate, pH 9.5. In the washing procedure the supernatant was carefully removed while the dynabeads were immobilized at the wall of the reaction vial using a magnetic separator stand (MSS). The washed dynabeads were incubated with 100 μg truncated Aβ mutein oligomer in 100 mM sodium borate, pH 9.5. The sample was shaken for 20 min at 37° C. Then, the sample was diluted 1:2 with 100 mM sodium borate, pH 9.5+0.5% BSA and was shaken overnight at 37° C. The dynabeads carrying the immobilized truncated Aβ mutein oligomer were washed 2×5 min (again using the MSS) with 200 μl PBS and 2×5 min with 200 μl PBS, 0.1% BSA, and were finally resuspended in 0.2 ml PBS, 0.1% BSA, 0.02% sodium azide and centrifuged briefly. The washed dynabeads carrying the immobilized truncated Aβ mutein oligomer were stored at 4° C. until further use.

Affinity Purification of pMAb's from Mouse Plasma Samples

Reagents:

PBS (20 mM NaH₂PO₄, 140 mM NaCl, pH7.4)

PBST (PBS+0.05% Tween 20)

PBST+0.5% BSA

elution buffer: 0.58% CH₃COOH/140 mM NaCl

neutralization buffer: 2 M Tris/HCl; pH 8.5

dynabeads carrying immobilized truncated Aβ mutein oligomer

Procedure:

10 μl mouse plasma sample were diluted with 80 μl PBST+0.5% BSA. 10 μl dynabeads carrying immobilized truncated Aβ mutein oligomer were added. The immunoprecipitation was carried out by shaking overnight (˜20 h) at room temperature. The dynabeads were immobilized using the MSS. The supernatant was carefully removed and discarded, and the dynabeads were washed 1×5 min with 500 μl PBST, 1×5 min with 500 μl PBS and 1×3 min with 500 μl 2 mM NaH₂PO₄, 14 mM NaCl, pH 7.5. After the last removal of the washing buffer, the reaction vials were once again centrifuged and remaining liquid was carefully and thoroughly removed. The dynabeads were suspended in 25 μl elution buffer and shaken for 2 min at room temperature. The reaction vials were centrifuged for 15 s at 4000 rpm, placed back in the MSS and supernatant (i.e. the eluate) was carefully removed and added to 975 μl PBST+0.5% BSA. 1 μl neutralization buffer was added and the sample mixed immediately for about 2-3 s. The binding of the affinity-purified polyclonal antibodies to Aβ globulomer was confirmed via direct ELISA.

### Example 5C: Analysis of Antibody Selectivity Via Dot Blot

In order to characterize the selectivity of the Aβ(20-42) mutein globulomer induced immune response, the affinity-purified polyclonal antisera were tested for binding to different Aβ forms. To this end, serial dilutions of the individual Aβ forms ranging from 100 pmol/μl to 0.00001 pmol/μl in PBS supplemented with 0.2 mg/ml BSA were prepared. 1 μl of each dilution was blotted onto a nitrocellulose membrane. Detection was performed by incubating with the corresponding affinity purified polyclonal antibodies response (0.2 μg/ml) followed by immunostaining with peroxidase-(POD-)conjugated IgG (anti-mouse-POD for antisera from mice, anti-rabbit-POD for rabbit antisera) and BM Blue POD Substrate (Roche).

Aβ-Standards for Dot-Blot:

1. Aβ(12-42) globulomer

Aβ(12-42) globulomer was prepared as described in Reference example 4

2. Aβ(1-42) globulomer

Aβ(1-42) globulomer was prepared as described in Reference example 3

3. Aβ(20-42) globulomer

Aβ(20-42) globulomer was prepared as described in Reference example 5.

4. Aβ(1-40) monomer, 0.1% NaOH

Aβ(1-40) monomer was prepared as described in Reference example 1.

5. Aβ(1-42) monomer, 0.1% NaOH

Aβ(1-42) monomer, 0.1% NaOH was prepared as described in Reference example 2

6. Aβ(1-42) fibrils

Aβ(1-42) fibrils were prepared as described in Reference example 6

7. sAPPα

sAPPα was prepared as described in Reference example 7

Materials for Dot Blot:

Serial dilution of Aβ-standards (see above 1. to 7.) in 20 mM NaH₂PO₄, 140 mM NaCl, pH 7.4+0.2 mg/ml BSA to obtain concentrations of: 100 pmol/μl, 10 pmol/μl, 1 pmol/μl, 0.1 pmol/μl, 0.01 pmol/μl, 0.001 pmol/μl, 0.0001 pmol/μl, and 0.00001 pmol/μl.

Nitrocellulose: Trans-Blot Transfer medium, Pure Nitrocellulose Membrane (0.2 μm); BIO-RAD

Anti-mouse-POD: cat no: 715-035-150 (Jackson Immuno Research)

Detection reagent: BM Blue POD Substrate, precipitating, cat no: 11442066001 (Roche)

Bovine serum albumin (BSA): cat no: 11926 (Serva)

Blocking reagent: 5% low fat milk in TBS

Buffer solutions:
TBS: 25 mM Tris/HCl buffer pH 7.5+150 mM NaCl
TTBS: 25 mM Tris/HCl-buffer pH 7.5+150 mM NaCl+0.05% Tween 20
PBS+0.2 mg/ml BSA: 20 mM NaH₂PO₄ buffer pH 7.4+140 mM NaCl+0.2 mg/ml BSA

Antibody solution I: 0.2 μg/ml antibody in 20 ml 1% low fat milk in TBS
Antibodies:
   Anti-Aβmouse monoclonal antibody clone 6E10; conc.: 1 mg/ml; cat. no.: SIG-39320 (Covance); stored at −80° C.
   Affinity-purified murine polyclonal anti-Aβ antibodies from example 5B-1, stored at −80° C.

Antibody solution II: for detection of mouse antibodies: 1:5000 dilution of anti-mouse-POD in 1% low fat milk in TBS

Dot Blot Procedure:
1) 1 μl of each of the 8 concentrations of the different Aβ-standards (obtained by serial dilution) was dotted onto the nitrocellulose membrane in a distance of approximately 1 cm from each other.
2) The dots of Aβ-standards were allowed to dry on the nitrocellulose membrane on air for at least 10 min at room temperature (=dot blot)
3) Blocking: The dot blot was incubated with 30 ml 5% low fat milk in TBS for 1.5 h at room temperature.
4) Washing: The blocking solution was discarded and the dot blot was incubated under shaking with 20 ml TTBS for 10 min at room temperature.
5) Antibody solution I: The washing buffer was discarded and the dot blot was incubated with antibody solution I for 2 h at room temperature
6) Washing: The antibody solution I was discarded and the dot blot was incubated under shaking with 20 ml TTBS for 10 min at room temperature. The washing solution was discarded and the dot blot was incubated under shaking with 20 ml TTBS for 10 min at room temperature. The washing solution was discarded and the dot blot was incubated under shaking with 20 ml TBS for 10 min at room temperature.
7) Antibody solution II: The washing buffer was discarded and the dot blot was incubated with antibody solution II for 1h at room temperature.
8) Washing: The antibody solution II was discarded and the dot blot was incubated under shaking with 20 ml TTBS for 10 min at room temperature. The washing solution was discarded and the dot blot was incubated under shaking with 20 ml TTBS for 10 min at room temperature. The washing solution was discarded and the dot blot was incubated under shaking with 20 ml TBS for 10 min at room temperature.
9) Development: The washing solution was discarded. The dot blot was developed with 7.5 ml BM Blue POD Substrate for 5 min. The development was stopped by intense washing of the dot blot with H₂O pH 5.3 (pH adjusted with sodium dihydrogenphosphate salt crystals).
10) Quantitative evaluation was done based on a densitometric analysis of the dot intensity using a GS800 densitometer (BioRad) and software package Quantity one, Version 4.5.0 (BioRad). Only dots were evaluated that had a relative density of greater than 20% of the relative density of the last optically unambiguously identified dot of the Aβ(20-42) globulomer. This threshold value was determined for every dot blot independently. The calculated value indicates the relation between recognition of Aβ(20-42) globulomer and the respective Aβ form for the given antibody.

Dot blot analysis was performed with different murine monoclonal (m6E10) and polyclonal anti-Aβ antibodies. Polyclonal anti-Aβ antibodies were obtained by active immunization of mice with truncated Aβ mutein oligomers followed by affinity purification (see example 5). The individual Aβ forms were applied in serial dilutions and incubated with the respective antibodies for immune reaction (1=Aβ(1-42) globulomer; 2=Aβ(20-42) globulomer; 3=Aβ(1-40) monomer, 0.1% NaOH; 4=Aβ(1-42) monomer, 0.1% NaOH; 5=Aβ(1-42) fibril preparation; 6=sAPPα (Sigma) (first dot: 1 pmol); 7=Aβ(12-42) globulomer). Results are shown in table 3.

TABLE 3
Dot blot quantification data of mouse polyclonal antibodies
A) Immunization with truncated Aβ E22A mutein oligomer
(mouse, Alum adjuvant)

| | Polyclonal antibodies of mouse # | | | |
|---|---|---|---|---|
| ANTIGEN | 1 | 2 | 3 | 4 |
| Aβ(1-42) globulomer | 0.86 | >10 | >100 | >100 |
| Aβ(12-42) globulomer | 2.82 | 1.97 | 1.98 | 2.61 |
| Aβ(20-42) globulomer | 1 | 1 | 1 | 1 |
| Aβ(1-40) monomer | 76.55 | >10 | >100 | >100 |
| Aβ(1-42) monomer | 36.45 | >10 | >100 | >100 |
| Aβ(1-42) fibril | 269.51 | >10 | >100 | >100 |
| sAPPα | 1.01 | >10 | >1 | >1 |

B) Immunization with truncated Aβ E22A mutein oligomer
(mouse, without adjuvant)

| | Polyclonal antibodies of mouse # | | | |
|---|---|---|---|---|
| ANTIGEN | 1 | 2 | 3 | 4 |
| Aβ(1-42) globulomer | >1000 | >1000 | >10 | >10 |
| Aβ(12-42) globulomer | 2.43 | 2.54 | 1.23 | 0.79 |
| Aβ(20-42) globulomer | 1 | 1 | 1 | 1 |
| Aβ(1-40) monomer | >1000 | >1000 | >10 | >10 |
| Aβ(1-42) monomer | 1950.71 | >1000 | >10 | >10 |
| Aβ(1-42) fibril | >1000 | >1000 | >10 | >10 |
| sAPPα | >10 | >10 | 1-10 | >0.38 |

C) Immunization with truncated Aβ F20G, E22A mutein oligomer
(mouse, Alum adjuvant)

| | Polyclonal antibodies of mouse # | | |
|---|---|---|---|
| ANTIGEN | 1 | 2 | 3 |
| Aβ(1-42) globulomer | 4.91 | 0.01 | 0.05 |
| Aβ(12-42) globulomer | 1.61 | 0.30 | 0.23 |
| Aβ(20-42) globulomer | 1 | 1 | 1 |
| Aβ(1-40) monomer | >10 | 2.81 | 13.41 |
| Aβ(1-42) monomer | >10 | >10 | 0.44 |
| Aβ(1-42) fibril | >10 | 15.56 | >10 |
| sAPPα | 0.29 | 1-10 | 0.001 |

D) Immunization with truncated Aβ G25V mutein oligomer
(mouse, Alum adjuvant)

| | Polyclonal antibodies of mouse # | |
|---|---|---|
| ANTIGEN | 1 | 2 |
| Aβ(1-42) globulomer | >100 | >10 |
| Aβ(12-42) globulomer | >100 | 0.72 |
| Aβ(20-42) globulomer | 1 | 1 |
| Aβ(1-40) monomer | >100 | >10 |
| Aβ(1-42) monomer | >100 | >10 |
| Aβ(1-42) fibril | >100 | >10 |
| sAPPα | >1 | 1-10 |

E) Immunization with truncated Aβ E22V mutein oligomer
(mouse, Alum adjuvant)

| | Polyclonal antibodies of mouse # | | | |
|---|---|---|---|---|
| ANTIGEN | 1 | 2 | 3 | 4 |
| Aβ(1-42) globulomer | 3.51 | 1.27 | 233.46 | 49.86 |
| Aβ(12-42) globulomer | 0.68 | 1.82 | 3.65 | 4.99 |
| Aβ(20-42) globulomer | 1 | 1 | 1 | 1 |
| Aβ(1-40) monomer | 286.00 | >100 | >100 | >100 |
| Aβ(1-42) monomer | 19.78 | 31.74 | 272.33 | >100 |
| Aβ(1-42) fibril | 172.65 | 125.59 | >100 | >100 |
| sAPPα | 0.092 | 0.039 | 1 | >1 |

F) Immunization with truncated Aβ E22A, G25A mutein oligomer
(mouse, Alum adjuvant)

| | Polyclonal antibodies of mouse # | | | |
|---|---|---|---|---|
| ANTIGEN | 1 | 2 | 3 | 4 |
| Aβ(1-42) globulomer | 1323.08 | 2171.70 | >1000 | 1864.35 |
| Aβ(12-42) globulomer | 1.74 | 5.32 | 2.75 | 1.92 |
| Aβ(20-42) globulomer | 1 | 1 | 1 | 1 |
| Aβ(1-40) monomer | >1000 | 3203.50 | 3899.23 | 3119.49 |
| Aβ(1-42) monomer | 1063.72 | 395.32 | 4406.81 | 724.17 |
| Aβ(1-42) fibril | >1000 | 2687.77 | >1000 | 2896.99 |
| sAPPα | 43.11 | 31.29 | 45.86 | 72.75 |

G) Immunization with truncated Aβ E22A, S26A mutein oligomer
(mouse, Alum adjuvant)

| | Polyclonal antibodies of mouse # | | | |
|---|---|---|---|---|
| ANTIGEN | 1 | 2 | 3 | 4 |
| Aβ(1-42) globulomer | 4.45 | 0.09 | 76.46 | >1 |
| Aβ(12-42) globulomer | 0.46 | 0.18 | 2.12 | 1.39 |
| Aβ(20-42) globulomer | 1 | 1 | 1 | 1 |
| Aβ(1-40) monomer | >10 | 5.96 | >100 | >1 |
| Aβ(1-42) monomer | 9.98 | 0.40 | 408.41 | >1 |
| Aβ(1-42) fibril | 2.37 | 0.01 | >100 | >1 |
| sAPPα | 1-10 | 0.0047 | >1 | 10-100 |

H) Immunization with truncated Aβ E22F mutein oligomer
(mouse, Alum adjuvant)

| | Polyclonal antibodies of mouse # | |
|---|---|---|
| ANTIGEN | 1 | 2 |
| Aβ(1-42) globulomer | 350.78 | 3116.21 |
| Aβ(12-42) globulomer | 1.35 | 1.57 |
| Aβ(20-42) globulomer | 1 | 1 |
| Aβ(1-40) monomer | >1000 | 528.23 |
| Aβ(1-42) monomer | >1000 | 812.97 |
| Aβ(1-42) fibril | 87.19 | 11.56 |
| sAPPα | 8.58 | 83.83 |

The dot-blot results show that immunization of mice with truncated Aβ mutein oligomers elicits a highly selective immune response for the Aβ globulomer epitope, which was also shown previously with wild type Aβ(20-42) globulomer. In the dot blot, the recognition of the polyclonal immune response was tested against the wild type Aβ(20-42) globulomer which presents the globulomer epitope as present in the brain of Alzheimer's disease patients. We presume that truncated Aβ mutein oligomers do not occur in the human body. However, immunization with the truncated Aβ mutein oligomers elicited an immune response that, as desired, was still able to recognize the wild type Aβ globulomer epitope. An active immunization with truncated Aβ mutein oligomers can thus be expected to be effective in reversing cognitive deficits in Alzheimer's disease transgenic mouse models because the polyclonal antisera dot blot profile of the elicited antibody response will be comparable to that of a response elicited by an active immunization with wild type Aβ(20-42) globulomer regarding the recognition of globulomer epitopes in vivo. The latter has been proven to reverse cognitive deficits in an object recognition task.

### Example 6: Determination of Cross-Reaction with Pf-4 in Cynomolgus Monkey Plasma Via Aligned Sandwich-ELISA

### Example 6A: Pf-4 Cross-Reactivity of Polyclonal Mouse Antibodies Affinity-Purified Via Sepharose Beads

Materials:

F96 Cert. Maxisorp NUNC-Immuno Plate cat. no. 439454

Binding Antibodies in Experiment:
polyclonal antibodies affinity-purified via sepharose beads from mouse plasma samples after immunization with different truncated Aβ mutein oligomers from example 5B-1
commercially available reference anti-PF4 antibody: monoclonal anti-HPF4 antibody (Abcam cat. no.: ab49735)

Coating buffer: 100 mM sodium hydrogen carbonate; pH 9.6

Blocking reagent for ELISA; Roche Diagnostics GmbH cat. no. 1112589

PBST buffer: 20 mM NaH₂PO₄; 140 mM NaCl; 0.05% Tween 20; pH 7.4 PBST+0.5% BSA buffer: 20 mM NaH₂PO₄; 140 mM NaCl; 0.05% Tween 20; pH 7.4+0.5% BSA; Serva cat. no. 11926

Cynomolgus plasma: Cynomolgus EDTA plasma pool from 13 different donors; stored at −30° C.

Trypsin inhibitor: Sigma cat. no. T7902

Aligning antibody: anti-mouse IgG (Fc specific; produced in goat; Sigma cat. no.: M3534; 2.3 mg/ml; stored at −20° C.

Detection antibody: polyclonal rabbit anti-PF-4 antibody pRAb-HPF4; 0.5 mg/ml; Abcam cat. no. ab9561

Label reagent: anti-rabbit-POD conjugate; Jackson ImmunoResearch Ltd. cat. no. 111-036-045

Staining Solution: 42 mM TMB (Roche Diagnostics GmbH cat. no.: 92817060) in DMSO; 3% H₂O₂ in water; 100 mM sodium acetate, pH 4.9

Stop solution: 2 M sulfonic acid

Preparation of Reagents:

Aligning antibody: The aligning antibody was diluted to 10 μg/ml in coating buffer.

Blocking solution: Blocking reagent was dissolved in 100 ml water to prepare the blocking stock solution and aliquots of 10 ml were stored at −20° C. 3 ml blocking stock solution was diluted with 27 ml water for each plate to block.

Each binding antibody was diluted with PBST+0.5% BSA buffer to 3.16 μg/ml (stock solution). Dilution series of each affinity-purified polyclonal antibody preparation were prepared as follows:

| | Volume of | | Volume of | | Final antibody |
|---|---|---|---|---|---|
| No | antibody dilution | | PBST + 0.5% BSA buffer | | concentration |
| 1 | 250 μl stock solution | | 0 | ml | 3160 ng/ml |
| 2 | 79 | μl (1) | 171 | μl | 1000 ng/ml |
| 3 | 79 | μl (2) | 171 | μl | 316 ng/ml |
| 4 | 79 | μl (3) | 171 | μl | 100 ng/ml |
| 5 | 79 | μl (4) | 171 | μl | 31.6 ng/ml |
| 6 | 79 | μl (5) | 171 | μl | 10 ng/ml |
| 7 | 79 | μl (6) | 171 | μl | 3.16 ng/ml |
| 8 | 0 | μl | 250 | μl | buffer only |

Cynomolgus Plasma:

5 ml Cynomolgus plasma pool were centrifuged for 10 min at 10000 g. 4.5 ml of the supernatant was removed and diluted with 40.5 ml PBST+0.5% BSA (=1:10 dilution). Then 450 μl 10 mg/ml trypsin inhibitor in H2O were added. After incubation for 10 min at room temperature the sample was filtrated through a 0.22 μm filter (Millipore cat. no. SLGS0250S).

Label Reagent:

Anti-rabbit-POD conjugate lyophilised was reconstituted in 0.5 ml water. 500 μl glycerol was added and aliquots of 100 μl were stored at 20° C. for further use. The concentrated label reagent was diluted in PBST buffer. The dilution factor was 1:5000. The reagent was used immediately.

Binding Antibody Plate Setup. Numbers indicate final concentrations of binding antibody in ng/ml. Each concentration of each binding antibody was run in duplicate.

| | 1 | 2 | 3 | 4 | 5 | 6 | 7 | 8 | 9 | 10 | 11 | 12 |
|---|---|---|---|---|---|---|---|---|---|---|---|---|
| | polyclonal | | polyclonal | | polyclonal | | polyclonal | | polyclonal | | polyclonal | |
| | antibody | | antibody | | antibody | | antibody | | antibody | | antibody | |
| A | 3160 | 3160 | 3160 | 3160 | 3160 | 3160 | 3160 | 3160 | 3160 | 3160 | 3160 | 3160 |
| B | 1000 | 1000 | 1000 | 1000 | 1000 | 1000 | 1000 | 1000 | 1000 | 1000 | 1000 | 1000 |
| C | 316 | 316 | 316 | 316 | 316 | 316 | 316 | 316 | 316 | 316 | 316 | 316 |
| D | 100 | 100 | 100 | 100 | 100 | 100 | 100 | 100 | 100 | 100 | 100 | 100 |
| E | 31.6 | 31.6 | 31.6 | 31.6 | 31.6 | 31.6 | 31.6 | 31.6 | 31.6 | 31.6 | 31.6 | 31.6 |
| F | 10 | 10 | 10 | 10 | 10 | 10 | 10 | 10 | 10 | 10 | 10 | 10 |
| G | 3.16 | 3.16 | 3.16 | 3.16 | 3.16 | 3.16 | 3.16 | 3.16 | 3.16 | 3.16 | 3.16 | 3.16 |
| H | 0.0 | 0.0 | 0.0 | 0.0 | 0.0 | 0.0 | 0.0 | 0.0 | 0.0 | 0.0 | 0.0 | 0.0 |

Procedure:
1. 100 μl of the aligning antibody solution per well were applied and incubated overnight at 6° C.
2. The antibody solution was discarded and the wells were washed three times with 250 μl PBST-buffer.
3. 265 μl blocking solution per well were added and incubated 2 h at room temperature.
4. The blocking solution was discarded and the wells were washed three times with 250 μl PBST buffer.
5. After preparation of the dilution series of each binding antibody, 100 μl per well of these antibody dilutions were applied to the plate. The plate was incubated 2 h at room temperature.
6. The antibody solutions were discarded and the wells were washed three times with 250 μl PBST buffer.
7. 100 μl 1:10 dilution of cynomolgus plasma per well were added and incubated 2 h at room temperature.
8. The plasma solution was discarded and the wells were washed three times with 250 μl PBST buffer.
9. 100 μl primary antibody solution per well were added and incubated 1 h at room temperature.
10. The primary antibody solution was discarded and the wells were washed three times with 250 μl PBST buffer.
11. 200 μl label reagent per well were added and incubated 1 h at room temperature.
12. The label reagent was discarded and the wells were washed three times with 250 μl PBST buffer.
13. 100 μl of TMB solution were added to each well.
14. Plate color was monitored during development (5-15 min at ambient temperature) and the reaction was terminated by adding 50 μl/well of stop solution when an appropriate color had developed.
15. The absorbance at 450 nm was measured.

Data Analysis:

The binding antibody concentrations (X-values) were log-transformed using the equation: X=log(X). Data were plotted using the log-transformed X-values on the X-axis expressed as amounts of antibody (expressed in ng/ml). The OD (450 nm) value of the respective PBST blank in row H was subtracted from the values of the polyclonal mouse antibody dilution series of each column in row A-G. The resulting background corrected OD (450 nm) values were plotted on the Y-axis. The concentration effect curves were calculated from these data points by curve fitting using a non-linear regression “four parameter logistic equation” with a “least squares (ordinary) fit” fitting method (that equals the fitting method “sigmoidal dose-response (variable slope)”) using the Data analysis software package GraphPadPrism (Version 5.03; GraphPad Software Inc.). Curve fitting was performed for the sole purpose of data visualization but not as basis for any further calculations i.e. the area under the curve calculation. The area under the curve (AUC, or total peak area) was determined based on non-curve fitted data, the log-transformed X-values and the OD (450 nm) values in the measured range (final plasma dilutions from 3.16 ng/ml to 3160 ng/ml). The following calculation settings were used within the Data analysis software package GraphPadPrism (Version 5.03; GraphPad Software Inc.):
The baseline was set to Y=0.0.
Minimum peak height: Ignore peaks that are less than 10% of the distance from minimum to maximum Y.
Peak direction: By definition, all peaks must go above the baseline.

For each individual antibody a PF4 discrimination factor was calculated using the commercially available anti-HPF4 antibody (Abcam cat. no.: ab49735) as a reference antibody for PF4 recognition, wherein

$$[ PF 4 discrimination factor ] = \frac{[ total peak area of anti - HPF 4 antibody ab 49735 ]}{[ total peak area of antibody to be determined ]}$$

Results of example 6A are shown tables 4A, 4B, and 4C.

TABLE 4A-C
AUC (or total peak area)
calculated from log-transformed data
A

| | | | anti-HPF4 | AUC |
|---|---|---|---|---|
| experim. 1 | Adjuvant | AUC | AUC mean | Ratio |
| anti-HPF4 | n.a. | 2.939 | 2.9 | 1.00 |
| anti-HPF4 | n.a. | 2.631 | | |
| mMAb 7C6 | n.a. | 1.577 | | 1.77 |
| mMAb 4D10 | n.a. | 0.127 | | 21.93 |
| mouse 1 (wt) | CFA | 0.512 | | 5.44 |
| mouse 2 (wt) | CFA | 0.610 | | 4.57 |
| mouse 3 (wt) | CFA | 2.993 | | 0.93 |
| mouse 4 (wt) | CFA | 0.675 | | 4.13 |
| mouse 37 (E22A) | CFA | 0.115 | | 24.15 |
| mouse 38 (E22A) | CFA | 0.053 | | 52.33 |
| mouse 39 (E22A) | CFA | 0.097 | | 28.57 |
| mouse 40 (E22A) | CFA | 0.083 | | 33.57 |
| mouse 73 (E22A) | CFA | 0.017 | | 159.23 |
| mouse 74 (E22A) | CFA | 0.002 | | 1274.02 |
| mouse 75 (E22A) | CFA | 0.013 | | 217.58 |
| mouse 76 (E22A) | CFA | 0.036 | | 76.36 |
| mouse 41 (D23A) | CFA | 0.356 | | 7.83 |
| mouse 42 (D23A) | CFA | 0.110 | | 25.41 |
| mouse 43 (D23A) | CFA | 0.098 | | 28.28 |
| mouse 44 (D23A) | CFA | 0.045 | | 61.71 |
| mouse 49 (S26A) | CFA | 3.099 | | 0.90 |
| mouse 50 (S26A) | CFA | 0.696 | | 4.00 |
| mouse 51 (S26A) | CFA | 0.352 | | 7.92 |
| mouse 52 (S26A) | CFA | 0.143 | | 19.43 |

B

| | Adjuvant | AUC | AUC Ratio |
|---|---|---|---|
| experim. 1 | | | |
| anti-HPF4 | n.a | 3.784 | 1.00 |
| mMAb 7C6 | n.a. | 2.882 | 1.31 |
| mMAb 4D10 | n.a. | 0.243 | 15.57 |
| m13 WT | Alum | 1.287 | 2.94 |
| m14 WT | Alum | 3.557 | 1.06 |
| m15 WT | Alum | 3.999 | 0.95 |
| experim. 2 | | | |
| anti-HPF4 | n.a. | 3.844 | 1.00 |
| m17 E22A | Alum | 0.150 | 25.70 |
| m18 E22A | Alum | 0.228 | 16.88 |
| m19 E22A | Alum | 0.100 | 38.44 |
| m20 E22A | Alum | 0.069 | 55.61 |
| m21 E22A | Alum | 0.040 | 96.80 |
| experim. 3 | | | |
| anti-HPF4 | n.a. | 3.868 | 1.00 |
| m22 E22A | Alum | 0.112 | 34.66 |
| m23 E22A | Alum | 0.168 | 23.04 |
| m24 E22A | Alum | 0.152 | 25.43 |
| m25 G25A | Alum | 2.929 | 1.32 |
| m26 G25A | Alum | 0.250 | 15.47 |
| experim. 4 | | | |
| anti-HPF4 | n.a. | 3.637 | 1.00 |
| mMAb 7C6 | n.a. | 2.997 | 1.21 |
| mMAb 4D10 | n.a. | 0.248 | 14.64 |
| m28 G25A | Alum | 2.779 | 1.31 |
| m29 G25A | Alum | 1.167 | 3.12 |
| m31 G25A | Alum | 0.557 | 6.53 |

C

| | Adjuvant | AUC | AUC Ratio |
|---|---|---|---|
| experim. 1 | | | |
| anti-HPF4 | n.a. | 3.043 | 1.00 |
| m1 WT | Alum | 0.065 | 47.00 |
| m2 WT | Alum | 0.063 | 48.21 |
| m3 WT | Alum | 2.288 | 1.33 |
| m4 WT | Alum | 0.254 | 11.96 |
| m5 E22A | Alum | 0.018 | 167.66 |
| m6 E22A | Alum | 0.084 | 36.40 |
| m7 E22A | Alum | 0.029 | 104.00 |
| m8 E22A | Alum | 0.047 | 65.27 |
| m9 E22A—Alum | Alum | 0.064 | 47.47 |
| m10 E22A—Alum | Alum | 0.019 | 161.95 |
| m11 E22A—Alum | Alum | 0.125 | 24.40 |
| m12 E22A—Alum | Alum | 0.024 | 125.54 |
| m13 F20G, E22A | Alum | 0.057 | 53.42 |
| m14 F20G, E22A | Alum | 0.001 | 4642.97 |
| m15 F20G, E22A | Alum | 0.039 | 78.77 |
| m17 G25T | Alum | 0.076 | 40.11 |
| m18 G25T | Alum | 1.639 | 1.86 |
| m19 G25T | Alum | 0.026 | 116.46 |
| m20 G25T | Alum | 1.657 | 1.84 |
| m21 G25V | Alum | 0.022 | 140.75 |
| m22 G25V | Alum | 0.031 | 99.77 |
| m23 G25V | Alum | 0.020 | 148.87 |
| experim. 2 | | | |
| anti-HPF4 | n.a. | 2.862 | 1.00 |
| m24 G25V | Alum | 0.012 | 247.15 |
| m25 E22V | Alum | 0.053 | 54.39 |
| m26 E22V | Alum | 0.066 | 43.59 |
| m27 E22V | Alum | 0.061 | 47.21 |
| m28 E22V | Alum | 0.053 | 53.88 |
| m29 E22A, G25A | Alum | 0.040 | 70.82 |
| m30 E22A, G25A | Alum | 0.037 | 78.28 |
| m31 E22A, G25A | Alum | 0.101 | 28.22 |
| m32 E22A, G25A | Alum | 0.012 | 238.90 |
| m33 G25A, S26A | Alum | 0.141 | 20.37 |
| m34 G25A, S26A | Alum | 0.004 | 751.18 |
| m35 G25A, S26A | Alum | 0.043 | 66.56 |
| m36 G25A, S26A | Alum | 0.092 | 30.99 |
| m37 E22A, S26A | Alum | 0.047 | 61.06 |
| m38 E22A, S26A | Alum | 0.155 | 18.49 |
| m39 E22A, S26A | Alum | 0.039 | 74.03 |
| m40 E22A, S26A | Alum | 0.022 | 132.38 |
| m41 D23K | Alum | 0.112 | 25.67 |
| m43 S26L | Alum | 0.022 | 130.86 |
| m44 S26L | Alum | 0.328 | 8.72 |
| m45 E22F | Alum | 0.066 | 43.12 |
| m46 E22F | Alum | 0.041 | 70.04 |

### Example 6B: PF-4 Cross-Reactivity of Polyclonal Mouse Antibodies Affinity-Purified Via Magnetic Dynabeads

Materials and preparation of reagents correspond to those in example 6A, except from: Binding antibodies in experiment:
polyclonal antibodies affinity-purified via activated dynabeads from mouse plasma samples after immunization with different truncated Aβ mutein oligomers from example 5B-2
commercially available reference anti-PF4 antibody: monoclonal anti-HPF4 antibody (Abcam cat. no.: ab49735)

The samples obtained in example 5B-2 after affinity purification of mouse plasma via magnetic dynabeads had a pre-dilution of 1:100. This affinity purified plasma stock solution was used here for a further dilution series. Dilution series of each affinity-purified polyclonal antibody preparation were prepared as follows:

| | | Volume of | | | |
|---|---|---|---|---|---|
| | Volume of | PBST + 0.5% | | Further plasma | Final plasma |
| No | antibody dilution | BSA buffer | | dilution factor | dilution factor |
| 1 | 250 μl stock solution | 0 | ml | directly | 1:100 |
| | (1:100 pre-diluted) | | | | |
| 2 | 50 μl (1) | 200 | μl | 1:5 | 1:500 |
| 3 | 50 μl (2) | 200 | μl | 1:25 | 1:2500 |
| 4 | 50 μl (3) | 200 | μl | 1:125 | 1:12500 |

Cynomolgus plasma and label reagent were prepared as in example 6A.

Binding Antibody Plate Setup. Numbers indicate dilutions of binding antibodies. Each concentration of each binding antibody was run in duplicate.

| | 1 | 2 | 3 | 4 | 5 | 6 | 7 | 8 | 9 | 10 | 11 | 12 |
|---|---|---|---|---|---|---|---|---|---|---|---|---|
| | polyclonal | | polyclonal | | polyclonal | | polyclonal | | polyclonal | | polyclonal | |
| | antibody | | antibody | | antibody | | antibody | | antibody | | antibody | |
| A | 1:100 | 1:100 | 1:100 | 1:100 | 1:100 | 1:100 | 1:100 | 1:100 | 1:100 | 1:100 | 1:100 | 1:100 |
| B | 1:500 | 1:500 | 1:500 | 1:500 | 1:500 | 1:500 | 1:500 | 1:500 | 1:500 | 1:500 | 1:500 | 1:500 |
| C | 1:2500 | 1:2500 | 1:2500 | 1:2500 | 1:2500 | 1:2500 | 1:2500 | 1:2500 | 1:2500 | 1:2500 | 1:2500 | 1:2500 |
| D | 1:12500 | 1:12500 | 1:12500 | 1:12500 | 1:12500 | 1:12500 | 1:12500 | 1:12500 | 1:12500 | 1:12500 | 1:12500 | 1:12500 |
| E | 1:100 | 1:100 | 1:100 | 1:100 | 1:100 | 1:100 | 1:100 | 1:100 | 1:100 | 1:100 | PBST | PBST |
| F | 1:500 | 1:500 | 1:500 | 1:500 | 1:500 | 1:500 | 1:500 | 1:500 | 1:500 | 1:500 | PBST | PBST |
| G | 1:2500 | 1:2500 | 1:2500 | 1:2500 | 1:2500 | 1:2500 | 1:2500 | 1:2500 | 1:2500 | 1:2500 | PBST | PBST |
| H | 1:12500 | 1:12500 | 1:12500 | 1:12500 | 1:12500 | 1:12500 | 1:12500 | 1:12500 | 1:12500 | 1:12500 | PBST | PBST |

The procedure corresponds to that in example 6A.

Data Analysis:

The binding antibody dilution factors (X-values) were log-transformed using the equation: X=log(X). Data were plotted using the log-transformed X-values on the X-axis expressed as dilution of plasma (1:X). The OD (450 nm) value of the respective PBST blank was subtracted from the values of the plasma dilution series of each column. The resulting background corrected OD (450 nm) values were plotted on the Y-axis. The dilution effect curves were calculated from these data points by curve fitting using a non-linear regression “four parameter logistic equation” with a “least squares (ordinary) fit” fitting method (that equals the fitting method “sigmoidal dose-response (variable slope)”) using the Data analysis software package GraphPadPrism (Version 5.03; GraphPad Software Inc.). Curve fitting was performed for the sole purpose of data visualization but not as basis for any further calculations i.e. the area under the curve calculation. The area under the curve (AUC, or total peak area) was determined based on non-curve fitted data, the log-transformed X-values and the OD (450 nm) values in the measured range (final plasma dilutions factors from 1:100 to 1:12500). The following calculation settings were used within the Data analysis software package GraphPadPrism (Version 5.03; GraphPad Software Inc.):
The baseline was set to Y=0.0.
Minimum peak height: Ignore peaks that are less than 10% of the distance from minimum to maximum Y.
Peak direction: By definition, all peaks must go above the baseline.

### Reference Example 1

Aβ(1-40) Monomer (0.1% NaOH)

1 mg Aβ(1-40) (Bachem Inc., cat. no. H-1194) was dissolved in 232.6 μl 0.1% NaOH in H₂O (freshly prepared) (=4.3 mg/ml=1 nmol/1 μl) and immediately shaken for 30 sec. at room temperature to get a clear solution. The sample was stored at −20° C. for further use.

### Reference Example 2

Aβ(1-42) Monomer (0.1% NaOH)

1 mg Aβ(1-42) (Bachem Inc., cat. no. H-1368) were dissolved in 222.2 μl 0.1% NaOH in H₂O (freshly prepared) (=4.5 mg/ml=1 nmol/1 μl) and immediately shaken for 30 sec. at room temperature to get a clear solution. The sample was stored at −20° C. for further use.

### Reference Example 3

Aβ(1-42) Globulomer

The Aβ(1-42) synthetic peptide (H-1368, Bachem, Bubendorf, Switzerland) was suspended in 100% 1,1,1,3,3,3-hexafluoro-2-propanol (HFIP) at 6 mg/ml and incubated for complete solubilization under shaking at 37° C. for 1.5 h. The HFIP acts as a hydrogen-bond breaker and is used to eliminate pre-existing structural inhomogeneities in the Aβ peptide. HFIP was removed by evaporation in a SpeedVac and Aβ(1-42) resuspended at a concentration of 5 mM in dimethylsulfoxide and sonicated for 20 s. The HFIP-pre-treated Aβ(1-42) was diluted in phosphate-buffered saline (PBS) (20 mM NaH₂PO₄, 140 mM NaCl, pH 7.4) to 400 μM and 1/10 volume 2% sodium dodecyl sulfate (SDS) (in H2O) added (final concentration of 0.2% SDS). An incubation for 6 h at 37° C. resulted in the 16/20 kDa Aβ(1-42) globulomer intermediate. The 38/48 kDa Aβ(1-42) globulomer was generated by a further dilution with three volumes of H2O and incubation for 18 h at 37° C. After centrifugation at 3000 g for 20 min the sample was concentrated by ultrafiltration (30-kDa cut-off), dialysed against 5 mM NaH₂PO₄, 35 mM NaCl, pH 7.4, centrifuged at 10,000 g for 10 min and the supernatant comprising the 38/48 kDa Aβ(1-42) globulomer withdrawn.

### Reference Example 4

Aβ(12-42) Globulomer

2 ml of the Aβ(1-42) globulomer preparation of reference example 3 were admixed with 38 ml buffer (5 mM sodium phosphate, 35 mM sodium chloride, pH 7.4) and 150 μl of a 1 mg/ml GluC endoproteinase (Roche) in water. The reaction mixture was stirred for 6 h at RT, and a further 150 μl of a 1 mg/ml GluC endoproteinase (Roche) in water were subsequently added. The reaction mixture was stirred at RT for another 16 h, followed by addition of 8 μl of a 5 M DIFP solution. The reaction mixture was concentrated to approx. 1 ml via a 15 ml 30 kDa Centriprep tube. The concentrate was admixed with 9 ml of buffer (5 mM sodium phosphate, 35 mM sodium chloride, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 1% strength SDS solution in water. The sample was centrifuged at 10,000 g for 10 min and the Aβ(12-42) globulomer supernatant was withdrawn.

### Reference Example 5

Aβ(20-42) Globulomer 1.59 ml of the Aβ(1-42) globulomer preparation of reference example 3 were admixed with 38 ml of buffer (50 mM MES/NaOH, pH 7.4) and 200 μl of a 1 mg/ml thermolysin solution (Roche) in water. The reaction mixture was stirred at RT for 20 h. Then 80 μl of a 100 mM EDTA solution, pH 7.4, in water were added and the mixture was furthermore adjusted to an SDS content of 0.01% with 400 μl of a 1% strength SDS solution. The reaction mixture was concentrated to approx. 1 ml via a 15 ml 30 kDa Centriprep tube. The concentrate was admixed with 9 ml of buffer (50 mM MES/NaOH, 0.02% SDS, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 2% strength SDS solution in water. The sample was centrifuged at 10,000 g for 10 min and the Aβ(20-42) globulomer supernatant was withdrawn.

### Reference Example 6

Aβ Fibrils 1 mg Aβ(1-42) (Bachem Inc. Catalog Nr.: H-1368) were dissolved in 500 μl aqueous 0.1% NH₄OH (Eppendorff tube) and the sample was stirred for 1 min at room temperature. The sample was centrifuged for 5 min at 10,000×g and the supernatant was withdrawn. 100 μl of this freshly prepared Aβ(1-42) solution were neutralized with 300 μl 20 mM NaH₂PO₄; 140 mM NaCl, pH 7.4. The pH was adjusted to pH 7.4 with 1% HCl. The sample was incubated for 24 h at 37° C. and centrifuged (10 min at 10,000 g). The supernatant was discarded and the fibril pellet washed twice with 400 μl 20 mM NaH₂PO₄, 140 mM NaCl, pH 7.4 and then finally resuspended with 400 μl of 20 mM NaH₂PO₄; 140 mM NaCl, pH 7.4 by vortexing for 1 min.

### Reference Example 7

sAPPα

Supplied from Sigma (cat. no. S9564; 25 μg in 20 mM NaH2PO4; 140 mM NaCl; pH 7.4). The sAPPα was diluted with 20 mM NaH₂PO₄, 140 mM NaCl, pH 7.4, 0.2 mg/ml BSA to 0.1 mg/ml (=1 pmol/μl).

What is claimed is:

1-103. (canceled)

104. An immunogenic product which comprises an N-terminally truncated form of an Aβ-mutein oligomer, which is obtainable by limited proteolytic digestion of a non-truncated Aβ mutein oligomer, which non-truncated Aβ mutein oligomer comprises a plurality of amino acid sequences, derived from the Aβ sequence according SEQ ID NO:1 by one or two amino acid substitutions:
i) wherein said one or two amino acid substitutions are performed in any one of the amino acid sequence positions F20, A21, E22, D23, V24, G25 and S26, and
ii) is reactive with monoclonal antibody 4D10 obtainable from a hybridoma designated by American Type Culture Collection deposit number PTA-7405.

105. The product of claim 104, wherein the monoclonal antibody binds the product with K_{D} of 1×10⁻⁶ M or greater affinity.

106. The product of claim 104, wherein the monoclonal antibody binds the product with K_{D} of 1×10⁻⁷ M or greater affinity.

107. The product of claim 104, wherein the one or two amino acid substitutions are performed in any one of the amino acid sequence positions F20, E22, D23, G25 and S26.

108. The product of claim 104, which comprises one or more single amino acid substitutions selected from the group consisting of: E22A, E22F, E22V, D23A, D23K, G25A, G25V, G25T, S26A and S26L;

109. The product of claim 104, which comprises one or more double amino acid substitutions selected from the group consisting of: (F20G, E22A), (E22A, G25A), (E22A, S26A), and (G25A, S26A).

110. The product of claim 104, capable of eliciting an polyclonal antiserum, wherein the polyclonal antiserum has an affinity to an Aβ globulomer which is at least 2 times greater than the affinity of the antiserum to at least one Aβ form selected from the group consisting of monomeric Aβ(1-42), monomeric Aβ(1-40), fibrillomeric Aβ(1-42), and fibrillomeric Aβ(1-40).

111. The product of claim 104, capable of eliciting an polyclonal antiserum, wherein the polyclonal antiserum has an affinity to an Aβ globulomer which is at least 100000 times greater than the affinity of the antiserum to at least one Aβ form selected from the group consisting of Aβ(1-42) globulomers, Aβ(12-42) globulomers and Aβ(20-42) globulomers.

112. The product of claim 104, capable of eliciting an polyclonal antiserum, wherein the polyclonal antiserum has an affinity to an Aβ(20-42) globulomer which is at least 2 times greater than the affinity of the antiserum to at least one Aβ globulomer selected from the group consisting of Aβ(1-42) globulomer and Aβ(12-42) globulomer.

113. The product of claim 104, capable of eliciting an polyclonal antiserum, wherein the polyclonal antiserum has an affinity to an Aβ(20-42) globulomer which is at least 100000 times greater than the affinity of the antiserum to at least one Aβ globulomer selected from the group consisting of Aβ(1-42) globulomer and Aβ(12-42) globulomer.

114. The product of claim 104 that is further characterized by one or more of the following features:
(a) at least part of said amino acid sequence forms a loop;
(b) the amino acid sequence portions of the product corresponding to F₁₉F₂₀A₂₁ (SEQ ID NO: 8) and A₃₀I₃₁I₃₂ (SEQ ID NO: 9) are in anti-parallel orientation;
(c) part of the amino acid sequence forms a loop comprising a sequence V₂₄G₂₅S₂₆N₂₇ (SEQ ID NO: 10) or D₂₃V₂₄G₂₅S₂₆N₂₇K₂₈ (SEQ ID NO: 11).

115. The product of claim 114, wherein the loop is a β-hairpin loop.

116. The product of claim 104, which is an oligomer comprising 2 to 28 of said Aβ amino acid sequences.

117. The product of claim 104, which is obtainable by a process comprising the following steps:
(a) dissolving a mutated Aβ peptide comprising said mutated Aβ amino acid sequence in a solvent;
(b) adding an amphipatic agent to the solution of the mutated Aβ peptide; and
(c) incubating the resulting mixture to form the oligomer; and
(d) proteolytically cleaving the oligomer.

118. The product of claim 104 that is further characterized by one or more of the following features:
(a) a first amino acid sequence L^{A}₃₄M^{A}₃₅V^{A}₃₆G^{A}₃₇G^{A}₃₈ (SEQ ID NO: 5) is in parallel orientation to a second amino acid sequence L^{B}₃₄M^{B}₃₅V^{B}₃₆G^{B}₃₇G^{B}₃₈ (SEQ ID NO: 5), wherein the interproton distance for at least one atom pair selected from the group consisting of M^{A}₃₅(NH)—V^{B}₃₆(NH), G^{A}₃₇(NH)-G^{B}₃₈(NH), L^{A}₃₄(NH)-L^{B}₃₄(CγH₃), M^{A}₃₅(NH)—V^{B}₃₆(CγH₃) is 1.8 to 6.5 Angstroms;
(b) a first amino acid sequence G^{A}₃₃L^{A}₃₄M^{A}₃₅V^{A}₃₆G^{A}₃₇G^{A}₃₈V^{A}₃₉ (SEQ ID NO: 6) is in parallel orientation to a second amino acid sequence G^{B}₃₃L^{B}₃₄M^{B}₃₅V^{B}₃₆G^{B}₃₇G^{B}₃₈V^{B}₃₉ (SEQ ID NO: 6), wherein the interproton distance for at least one atom pair selected from the group consisting of G^{A}₃₃(NH)-G^{B}₃₄(NH), M^{A}₃₅(NH)—V^{B}₃₆(NH), G^{A}₃₇(NH)-G^{B}₃₈(NH), L^{A}₃₄(NH)-L^{B}₃₄(CγH₃), M^{A}₃₅(NH)—V^{B}₃₆(CγH₃), G^{A}₃₈(NH)—V^{B}₃₉(CγH₃) and V^{A}₃₉(NH)—V^{B}₃₉(CγH₃) is 1.8 to 6.5 Angstroms; or
(c) the product comprises an inter-molecular parallel β-sheet between two Aβ amino acid sequences, wherein the inter-molecular parallel 3-sheet comprises a first amino acid sequence G^{A}₃₃L^{A}₃₄M^{A}₃₅V^{A}₃₆G^{A}₃₇G^{A}₃₈V^{A}₃₉ (SEQ ID NO: 7) and a second amino acid sequence G^{B}₃₃L^{B}₃₄M^{B}₃₅V^{B}₃₆G^{B}₃₇G^{B}₃₈V^{B}₃₉ (SEQ ID NO: 7), and wherein the atom pairs G^{A}₃₃(CO)-L^{B}₃₄(N), L^{B}₃₄(CO)-M^{A}₃₅(N), M^{A}₃₅(CO)—V^{B}₃₆(N), V^{B}₃₆(CO)-G^{A}₃₇(N), and G^{B}₃₇(CO)-G^{A}₃₈(N) are at a distance of 3.3+0.5 Δ, CO indicates the backbone oxygen atom, the phi (φ) angles of the residues range from −180 to −30 and psi (ψ) angles of the residues range from approximately 60 to 180 or from approximately −180 to −150.

119. The product of claim 104, wherein the mutated Aβ amino acid sequence is identical to a portion (X—Y) of a mutated amino acid sequence selected from the group consisting of:
(SEQ ID NO: 15)
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 16)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁F₂₂D₂₃V₂₄G₂₅S₂₆N₂₇K₂₈
G₂₉A₃₀I₃₁I₃₂G₃₄L₃₄M₃₅V₃₆G₃₇G₃₈V₃₉V₄₀I₄₁A₄₂T₄₃,
(SEQ ID NO: 17)
D₁A₂E₃F₄R₅H₆D₇S₈G₉Y₁₀E₁₁V₁₂H₁₃H₁₄Q₁₅K₁₈L₁₇V₁₈F₁₉F₂₀A₂₁V₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 18)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 19)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂K₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 20)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂L₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 21)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄V₂₅S₂₆N₂₇K₂₈
G₂₉A₃₀I₃₁I₃₂G₃₃L₃₄M₃₅V₃₆G₃₇G₃₈V₃₉V₄₀₁₄₁A₄₂T₄₃,
(SEQ ID NO: 22)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁E₂₂D₂₃V₂₄G₂₅S₂₆N₂₇K₂₈
G₂₉G₃₀I₃₁I₃₂G₃₃L₃₄M₃₅V₃₆G₃₇G₃₈V₃₉V₄₀I₄₁A₄₂T₄₃,
(SEQ ID NO: 23)
D₁A₂E₃F₄R₅H₆D₇S₈G₉Y₁₀E₁₁V₁₂H₁₃H₁₄Q₁₅K₁₆L₁₇V₁₈F₁₉G₂₀A₂₁A₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 24)
D₁A₂E₃F₄R₅H₆D₇S₈G₉Y₁₀E₁₁V₁₂H₁₃H₁₄Q₁₅K₁₆L₁₇V₁₈F₁₉A₂₀A₂₁E₂₂D₂₃V₂₄G₂₅S₂₆N₂₇K₂₈
G₂₉A₃₀A₃₁I₃₂G₃₃L₃₄M₃₅V₃₆G₃₇G₃₈V₃₉V₄₀I₄₁A₄₂T₄₃,
(SEQ ID NO: 25)
D₁A₂E₃F₄R₅H₆D₇S₈G₉Y₁₀E₁₁V₁₂H₁₃H₁₄Q₁₅K₁₆L₁₇V₁₈F₁₉C₂₀A₂₁E₂₂D₂₃V₂₄G₂₅S₂₆N₂₇K₂₈
G₂₉A₃₀C₃₁I₃₂G₃₃L₃₄M₃₅V₃₆G₃₇G₃₈V₃₉V₄₀I₄₁A₄₂T₄₃,
(SEQ ID NO: 26)
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁L₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 27)
D₁A₂E₃F₄R₅H₆D₇S₈G₉Y₁₀E₁₁V₁₂H₁₃H₁₄Q₁₅K₁₆L₁₇V₁₈F₁₉F₂₀L₂₁Q₂₂D₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 28)
D₁A₂E₃F₄R₅H₆D₇S₈G₉Y₁₀E₁₁V₁₂H₁₃H₁₄Q₁₅K₁₆L₁₇V₁₈F₁₉F₂₀Q₂₁E₂₂N₂₃V₂₄G₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
(SEQ ID NO: 29)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄A₂₅S₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃,
and
(SEQ ID NO: 30)
D₁A₂E₃F₄R₅H₆D₇S₈G₉Y₁₀E₁₁V₁₂H₁₃H₁₄Q₁₅K₁₆L₁₇V₁₈F₁₉F₂₀A₂₁A₂₂D₂₃V₂₄G₂₅A₂₆N₂₇K₂₈
G₂₉A₃₀I₃₁I₃₂G₃₃L₃₄M₃₅V₃₆G₃₇G₃₈V₃₉V₄₀I₄₁A₄₂T₄₃
with X being selected from the group consisting of the numbers, 4 to 18, 12 to 18, and 18; and with Y being selected from the group consisting of the numbers 33 to 43, 33 to 42, 33 to 41, and 33 and 40; or a crosslinked derivative thereof, wherein at least 2 non-contiguous residues of the amino acid sequence are covalently linked with each other.

120. The product of claim 119, wherein (X—Y) is selected from the group consisting of (4-42), (12-42), and (18-42).

121. A composition comprising a product as defined in claim 104, wherein the composition is a vaccine and optionally comprises a pharmaceutically acceptable excipient.

122. The composition of claim 120, wherein the pharmaceutically acceptable excipient is Complete Freund's Adjuvant (CFA) or an adjuvant comprising an aluminium salt.


SEQUENCE LISTING (31 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 16 aa, synthetic construct
  REGION 1..16: note = Description of Artificial Sequence: Synthetic peptide
VFFAEDVGSNKGAIIG

SEQ ID NO: 3 - protein, 22 aa, synthetic construct
  REGION 1..22: note = Description of Artificial Sequence: Synthetic peptide
VFFAEDVGSNKGAIIGLMVGGV

SEQ ID NO: 4 - protein, 28 aa, synthetic construct
  REGION 1..28: note = Description of Artificial Sequence: Synthetic peptide
VHHQKLVFFAEDVGSNKGAIIGLMVGGV

SEQ ID NO: 5 - protein, 5 aa, synthetic construct
  REGION 1..5: note = Description of Artificial Sequence: Synthetic peptide
LMVGG

SEQ ID NO: 6 - protein, 7 aa, synthetic construct
  REGION 1..7: note = Description of Artificial Sequence: Synthetic peptide
GLMVGGV

SEQ ID NO: 7 - protein, 7 aa, synthetic construct
  REGION 1..7: note = Description of Artificial Sequence: Synthetic peptide
GLMVGGV

SEQ ID NO: 8-9 - skipped (2 entries)

SEQ ID NO: 10 - protein, 4 aa, synthetic construct
  REGION 1..4: note = Description of Artificial Sequence: Synthetic peptide
VGSN

SEQ ID NO: 11 - protein, 6 aa, synthetic construct
  REGION 1..6: note = Description of Artificial Sequence: Synthetic peptide
DVGSNK

SEQ ID NO: 12 - protein, 70 aa, synthetic construct
  REGION 1..70: note = Description of Artificial Sequence: Synthetic polypeptide
EAEEDGDLQCLCVKTTSQVRPRHITSLEVIKAGPHCPTAQLIATLKNGRKICLDLQAPLYKKIIKKLLES

SEQ ID NO: 13 - protein, 43 aa, synthetic construct
  REGION 1..43: note = Description of Artificial Sequence: Synthetic polypeptide
DAEFRHDSGYEVHHQKLVAFAEDVGSNKGAIIGLMVGGVVIAT

SEQ ID NO: 14 - protein, 43 aa, synthetic construct
  REGION 1..43: note = Description of Artificial Sequence: Synthetic polypeptide
DAEFRHDSGYEVHHQKLVFAAEDVGSNKGAIIGLMVGGVVIAT

SEQ ID NO: 15 - protein, 43 aa, synthetic construct
  REGION 1..43: note = Description of Artificial Sequence: Synthetic polypeptide
DAEFRHDSGYEVHHQKLVFFAADVGSNKGAIIGLMVGGVVIAT

SEQ ID NO: 16 - protein, 43 aa, synthetic construct
  REGION 1..43: note = Description of Artificial Sequence: Synthetic polypeptide
DAEFRHDSGYEVHHQKLVFFAFDVGSNKGAIIGLMVGGVVIAT

SEQ ID NO: 17 - protein, 43 aa, synthetic construct
  REGION 1..43: note = Description of Artificial Sequence: Synthetic polypeptide
DAEFRHDSGYEVHHQKLVFFAVDVGSNKGAIIGLMVGGVVIAT

SEQ ID NO: 18 - protein, 43 aa, synthetic construct
  REGION 1..43: note = Description of Artificial Sequence: Synthetic polypeptide
DAEFRHDSGYEVHHQKLVFFALDVGSNKGAIIGLMVGGVVIAT

SEQ ID NO: 19 - protein, 43 aa, synthetic construct
  REGION 1..43: note = Description of Artificial Sequence: Synthetic polypeptide
DAEFRHDSGYEVHHQKLVFFAEKVGSNKGAIIGLMVGGVVIAT

SEQ ID NO: 20 - protein, 43 aa, synthetic construct
  REGION 1..43: note = Description of Artificial Sequence: Synthetic polypeptide
DAEFRHDSGYEVHHQKLVFFAELVGSNKGAIIGLMVGGVVIAT

SEQ ID NO: 21 - protein, 43 aa, synthetic construct
  REGION 1..43: note = Description of Artificial Sequence: Synthetic polypeptide
DAEFRHDSGYEVHHQKLVFFAEDVVSNKGAIIGLMVGGVVIAT

SEQ ID NO: 22 - protein, 43 aa, synthetic construct
  REGION 1..43: note = Description of Artificial Sequence: Synthetic polypeptide
DAEFRHDSGYEVHHQKLVFFAEDVGSNKGGIIGLMVGGVVIAT

SEQ ID NO: 23 - protein, 43 aa, synthetic construct
  REGION 1..43: note = Description of Artificial Sequence: Synthetic polypeptide
DAEFRHDSGYEVHHQKLVFGAADVGSNKGAIIGLMVGGVVIAT

SEQ ID NO: 24 - protein, 43 aa, synthetic construct
  REGION 1..43: note = Description of Artificial Sequence: Synthetic polypeptide
DAEFRHDSGYEVHHQKLVFAAEDVGSNKGAAIGLMVGGVVIAT

SEQ ID NO: 25 - protein, 43 aa, synthetic construct
  REGION 1..43: note = Description of Artificial Sequence: Synthetic polypeptide
DAEFRHDSGYEVHHQKLVFCAEDVGSNKGACIGLMVGGVVIAT

SEQ ID NO: 26 - protein, 43 aa, synthetic construct
  REGION 1..43: note = Description of Artificial Sequence: Synthetic polypeptide
DAEFRHDSGYEVHHQKLVFFQLDVGSNKGAIIGLMVGGVVIAT

SEQ ID NO: 27 - protein, 43 aa, synthetic construct
  REGION 1..43: note = Description of Artificial Sequence: Synthetic polypeptide
DAEFRHDSGYEVHHQKLVFFLQDVGSNKGAIIGLMVGGVVIAT

SEQ ID NO: 28 - protein, 43 aa, synthetic construct
  REGION 1..43: note = Description of Artificial Sequence: Synthetic polypeptide
DAEFRHDSGYEVHHQKLVFFQENVGSNKGAIIGLMVGGVVIAT

SEQ ID NO: 29 - protein, 43 aa, synthetic construct
  REGION 1..43: note = Description of Artificial Sequence: Synthetic polypeptide
DAEFRHDSGYEVHHQKLVFFAADVASNKGAIIGLMVGGVVIAT

SEQ ID NO: 30 - protein, 43 aa, synthetic construct
  REGION 1..43: note = Description of Artificial Sequence: Synthetic polypeptide
DAEFRHDSGYEVHHQKLVFFAADVGANKGAIIGLMVGGVVIAT

SEQ ID NO: 31 - protein, 6 aa, synthetic construct
  REGION 1..6: note = Description of Artificial Sequence: Synthetic peptide
HHHHHH